(19) United States
(12) Patent Application Publication
(10) Pub. No.: US 2026/0345770 A1
(43) Pub. Date: Nov. 5, 2026

(54) COMPOSITIONS AND METHODS FOR MODULATING IMMUNE RESPONSE

(71) Applicant: NOVAVAX, INC., Gaithersburg, MD (US)

(72) Inventors: Linda STERTMAN, Uppsala (SE); Berit CAROW, Uppsala (SE)

(21) Appl. No.: 19/647,607

(22) Filed: Apr. 14, 2026

Related U.S. Application Data
(60) Provisional application No. 63/788,358, filed on Apr. 14, 2025.

(51) Int. Cl. A61K 39/39; A61K 39/00; A61K 39/155; A61K 39/215; A61P 31/14; A61P 37/04

(52) U.S. Cl. CPC A61K 39/39; A61K 39/0011; A61K 39/155; A61K 39/215; A61P 31/14; A61P 37/04; A61K 2039/545; A61K 2039/55555; A61K 2039/55577; A61K 2039/572; A61K 2039/575

(57) ABSTRACT

The invention relates to saponin matrix adjuvants comprising Matrix-A and Matrix-C particles at fixed weight ratios between about 70:30 to 10:90 (Matrix-A:Matrix-C), specifically about 60:40, 59:41, 58:42, 57:43, 56:44, 55:45, 54:46, 53:47, 52:48, 51:49, or 50:50, in contrast to conventional 85:15 Matrix-M adjuvant. Matrix-A and Matrix-C are cage-like particles formed from chromatographically separated Quillaja saponin fractions with cholesterol and phosphatidylcholine. At the disclosed ratios, the compositions unexpectedly enhance lysosomal targeting, lysosomal membrane permeabilization, antigen cross-presentation, and activation of antigen-presenting cells, yielding at least about 30-100% higher frequencies of antigen-specific CD8 T cells producing IFN-γ under comparable dosing conditions. The saponin matrix compositions can be formulated with viral antigens (for example SARS-CoV-2 spike, RSV F0, PIV3, hMPV F) or tumor antigens (including defined tumor-associated antigens and neoantigens) to provide vaccines that induce protective antiviral immunity and therapeutic antitumor responses using manufacturing processes compatible with existing Matrix platforms.

### CROSS-REFERENCE TO RELATED APPLICATIONS

The present application claims priority to U.S. Provisional Patent Application No. 63/788,358, filed on Apr. 14, 2025, the contents of which is hereby incorporated by reference in its entirety.

### FIELD OF THE INVENTION

The present invention relates to immunology and vaccine technology, and more particularly to saponin-based adjuvant compositions comprising Matrix-A and Matrix-C particles of Quillaja saponaria saponins at defined fixed weight ratios, vaccine formulations that include such adjuvant compositions and one or more antigens, and methods of using these compositions and formulations to modulate immune responses, including enhancing antigen cross-presentation and antigen-specific CD8 T-cell responses, in human and veterinary subjects.

### BACKGROUND OF THE INVENTION

Saponins are a large family of plant-derived glycoconjugates that share a triterpene structure with a variety of glycoside side chains. Because of their amphipathic properties, saponins also have been used in a variety of other surfactant applications. Saponins have traditionally been used for making soaps and detergents based on being amphipathic. More recently, saponins are used for making adjuvants based on having potent immune-stimulating properties, as taught, for example, by Kensil et al., U.S. Pat. No. 5,057,540.

Saponins extracted from the bark of the South American soapbark tree Quillaja saponaria Molina contain a complex heterogeneous mixture of closely related saponins with structurally different glycosylation or acylation patterns that affect their biological activities. Without being bound by theory, differences in glycosylation and acylation can influence both potency and tolerability of saponin-containing formulations. Quillaja saponaria Molina saponins can have a high degree of glycosyl O-acylation, a low degree of glycosyl O-acylation, or no glycosyl O-acylation in their naturally occurring forms. Saponins also can be chemically modified, for example by partial or complete deacylation or degradation.

Saponins of Quillaja saponaria Molina in particular can have potent adjuvant activity, but also can be chemically unstable, show hemolytic activity, and be associated with immediate pain at injection sites. These liabilities can limit dose levels, formulation options, and clinical utility in some settings. Accordingly, there has been a need for adjuvant systems that retain the desired immune-stimulating properties of such saponins while reducing their instability and reactogenicity. Saponin preparations based on defined compositions of purified saponin fractions of Quillaja saponaria Molina are described, for example, by Cox et al., PCT/AU1995/000670 (WO96/011711).

Incorporating saponins of Quillaja saponaria Molina into particles including saponin and lipid can attenuate the chemical instability, hemolytic activity, and immediate pain when injected associated with saponins. In particular, lipid-containing particles can provide improved physical stability and a more favorable safety and reactogenicity profile while maintaining robust adjuvant activity. Specific examples of particles including saponin and lipid include saponin based particles and saponin based antigen-presenting particles, as taught, for example, by Stertman et al., Human Vaccines & Immunotherapeutics, 2023, 19(1):2189885, GSK's Liposome-based Adjuvant System 01 particles, as described, for example, in Didierlaurent et al., Expert Review of Vaccines, 2017, 16(1):55-63, and Army Liposome Formulation Q particles, as described, for example, by Alving et al., Expert Review of Vaccines, 2020, 19(3):279-292.

Novavax's Matrix-M adjuvant is a formulation that is manufactured using two purified extracts of saponins of Quillaja saponaria Molina, termed saponin Fraction-A and saponin Fraction-C, which are described in detail below. These fractions each contain multiple individual saponin species, but are standardized with respect to overall composition and biological activity. To make Matrix-M adjuvant, saponin Fraction-A and saponin Fraction-C are separately mixed with cholesterol and phosphatidylcholine, in the presence of the detergent Mega-10 to form dispersions of approximately 40 nm-sized stable cage-like structures, designated Matrix-A and Matrix-C particles. The Mega-10 detergent is removed by diafiltration. The Matrix-A and Matrix-C particles are provided in formulations in phosphate buffer solution including 137 mM sodium chloride, 2.7 mM potassium chloride, and 9.8 mM phosphate, at pH 7.2. These conditions yield particle preparations that are physically stable and suitable for combination with a variety of vaccine antigens. Unless otherwise indicated, references to “Matrix-M adjuvant” herein refer to such preparations including Matrix-A and Matrix-C particles in an aqueous buffer suitable for administration. The Matrix-M adjuvant is obtained by mixing the Matrix-A and Matrix-C particles at a fixed weight ratio of 85:15 of Matrix-A particles to Matrix-C particles.

Considering saponins in more detail, as noted above, saponin preparations based on defined compositions of purified saponin fractions of Quillaja saponaria Molina are described, for example, by Cox et al., PCT/AU1995/000670 (WO96/011711). Historically, formulation of saponins into particles was performed with a semi-purified, non-fractionated saponin extract from the bark of Quillaja saponaria Molina, termed Quil-A. This led to several of the benefits of the formulation of saponins into such particles being recognized. For example, formulation into particles enabled use of saponins at doses and in combinations that would not have been acceptable in non-particulate form. Ambitions to bring the technology further towards a possible product, such as an adjuvant for use in animal and human vaccines, prompted increased purification and characterization of the Quil-A extract. The tools available for separation and characterization of saponins at the time, during the mid to late 1980s, were reversed-phase high-performance liquid chromatography, also termed RP-HPLC, and thin layer chromatography, also termed TLC. The number of peaks revealed by RP-HPLC was numerous, while TLC revealed a few major bands. These analytical approaches allowed enrichment of subsets of structurally related saponins despite the overall complexity of the starting material. Saponin raw material was subjected to semi-preparative high-performance liquid chromatography, also termed HPLC, separations. By screening separated saponin materials for adjuvant activity in mice, it was found that the saponins of major interest were residing in one of the major TLC bands. Further separation of the saponins by HPLC and screening for adjuvant activity and structure forming ability resulted in the definition of sub-groups of saponin fractions with different and interesting features. These efforts ultimately led to the identification of discrete saponin fractions that could be reproducibly manufactured and formulated into lipid particles. The major findings are summarized in TABLE 1, in which present terminology and major components are included for clarity, and reference is made to what currently are termed Fraction-A, Fraction-B, and Fraction-C of Quillaja saponaria Molina saponins.

TABLE 1
Illustrative initial characterization of potentially useful
fractions of saponin materials obtained from Quil-A.

| | Adjuvant | | | | |
|---|---|---|---|---|---|
| | activity | Matrix | | Mw of | |
| | non- | structure | | major | Present |
| TLC | formulated | forming | Hemolytic | known | termi- |
| band | saponin) | ability | activity | component(s) | nology |
| B1 | Poor | Poor | Low | NA | NA |
| B2 | Potent | Good | Medium | 1988 | Fraction-C |
| B3 | Potent | Atypical | High | 2150 (2175 | Fraction-B |
| | | structures | | and 2019) | |
| B4 | Poor/none | Good | Low | 1862 | Fraction-A |

Over the years, the process for fractionation of saponin raw material into Fraction-A and Fraction-C has been developed and scaled up. These processes enable robust, reproducible manufacture of well-characterized saponin fractions suitable for incorporation into vaccine adjuvant formulations. For example, as described in Cox et al., PCT/AU1995/000670 (WO96/011711), Fractions-A, -B, and -C can be prepared from the lipophilic fraction obtained on chromatographic separation of the crude aqueous Quillaja Saponaria Molina extract on a SEP-PAK column and elution with 70% acetonitrile in water to recover the lipophilic fraction. This lipophilic fraction can then be separated by semipreparative HPLC with elution using a gradient of from 25% to 60% acetonitrile in acidic water. In certain implementations, Fraction-A is the fraction that is eluted at approximately 39% acetonitrile. Fraction-B is the fraction that is eluted at approximately 47% acetonitrile. Fraction-C is the fraction that is eluted at approximately 49% acetonitrile.

The mode of action of Matrix-M adjuvant is thought to involve the rapid and transient presence of the Matrix-A particles and Matrix-C particles present in Matrix-M adjuvant and activated innate immune cells in the lymph node draining the injection site. This early innate response is believed to contribute to the enhanced magnitude and quality of downstream adaptive immune responses, including CD4+ and CD8+ T cell responses. Intracellular events following the uptake of the Matrix-M adjuvant and their contribution to the generation of CD8+ response adjuvant effect remain largely unknown, and are the subject of ongoing investigation. Nonetheless, experimental data are consistent with Matrix-M promoting antigen cross-presentation and the induction of antigen-specific CD8+ T-cell responses, as described in further detail herein.

### SUMMARY OF THE INVENTION

The present invention provides saponin matrix compositions and vaccine formulations that employ a defined fixed weight ratio of Matrix-A particles to Matrix-C particles. These compositions are typically administered together with an antigen, such as a viral antigen (for example, SARS-CoV-2 S protein, gE/gI, PIV3, RSV F0, or hMPV F) or a tumor antigen (including TAAs or tumor neoantigens), to induce enhanced antigen cross-presentation, increased activation of antigen-presenting cells, and elevated antigen-specific CD8 T-cell responses, including IFN-γ-producing CD8 T cells, compared to conventional Matrix-M adjuvant formulations having an 85:15 Matrix-A:Matrix-C ratio. In various aspects, the invention thus encompasses the saponin matrix compositions themselves (optionally with antigen), methods of using such compositions for inducing production of IFN-γ by antigen-specific CD8 T cells, eliciting cross-presentation of antigens, treating cancer with tumor antigens, inducing adaptive immune responses against viral pathogens, and activating antigen-presenting cells, as well as associated pharmaceutical dosage forms and routes of administration suitable for human and veterinary use.

The present invention provides a saponin matrix composition. The saponin matrix composition includes Matrix-A particles and Matrix-C particles at a defined fixed weight ratio.

The present invention also provides a vaccine composition. The vaccine composition includes an antigen and a saponin matrix composition. The saponin matrix composition includes Matrix-A particles and Matrix-C particles at a defined fixed weight ratio.

The present invention also provides a method of administering an antigen to a subject in need thereof. The method includes administering to the subject an effective amount of an antigen and an effective amount of a saponin matrix composition. The saponin matrix composition includes Matrix-A particles and Matrix-C particles at a defined fixed weight ratio.

The present invention also provides a method of inducing an adaptive immune response in a subject, wherein the response is substantially independent of NLRP3 inflammasome activation. The method includes administering to the subject an effective amount of an antigen and an effective amount of a saponin matrix composition. The saponin matrix composition includes Matrix-A particles and Matrix-C particles at a defined fixed weight ratio.

The present invention also provides a method for inducing production of IFN-γ by antigen-specific CD8 T cells in a subject in need thereof. The method includes administering to the subject an effective amount of an antigen and an effective amount of a saponin matrix composition, thereby inducing production of IFN-γ by antigen-specific CD8 T cells in the subject. The saponin matrix composition includes Matrix-A particles and Matrix-C particles at a defined fixed weight ratio.

The present invention also provides a method of treatment for cancer in a subject in need thereof. The method includes administering to the subject an effective amount of a tumor antigen and an effective amount of a saponin matrix composition, thereby treating the cancer in the subject. The saponin matrix composition includes Matrix-A particles and Matrix-C particles at a defined fixed weight ratio.

The present invention also provides a method of inducing an adaptive immune response against a viral pathogen in a subject in need thereof. The method includes administering to the subject an effective amount of a viral antigen and an effective amount of a saponin matrix composition, thereby inducing an adaptive immune response against the viral pathogen in the subject. The saponin matrix composition includes Matrix-A particles and Matrix-C particles at a defined fixed weight ratio.

In at least some embodiments described above, the Matrix-A particles and the Matrix-C particles are present at a fixed weight ratio in the range of 70:30 to 10:90 of the Matrix-A particles to the Matrix-C particles, thereby modulating immune responses in a subject. In specific embodiments, the ratio of the Matrix-A particles to the Matrix-C particles is about 60:40, 59:41, 58:42, 57:43, 56:44, 55:45, 54:46, 53:47, 52:48, 51:49, or 50:50. In specific embodiments, the ratio of the Matrix-A particles to the Matrix-C particles is about: 60:40 to 59:41, 60:40 to 58:42, 60:40 to 57:43, 60:40 to 56:44, 60:40 to 55:45, 60:40 to 54:46, 60:40 to 53:47, 60:40 to 52:48, 60:40 to 51:49, 60:40 to 50:50, 59:41 to 58:42, 59:41 to 57:43, 59:41 to 56:44, 59:41 to 55:45, 59:41 to 54:46, 59:41 to 53:47, 59:41 to 52:48, 59:41 to 51:49, 59:41 to 50:50, 58:42 to 57:43, 58:42 to 56:44, 58:42 to 55:45, 58:42 to 54:46, 58:42 to 53:47, 58:42 to 52:48, 58:42 to 51:49, 58:42 to 50:50, 57:43 to 56:44, 57:43 to 55:45, 57:43 to 54:46, 57:43 to 53:47, 57:43 to 52:48, 57:43 to 51:49, 57:43 to 50:50, 56:44 to 55:45, 56:44 to 54:46, 56:44 to 53:47, 56:44 to 52:48, 56:44 to 51:49, 56:44 to 50:50, 55:45 to 54:46, 55:45 to 53:47, 55:45 to 52:48, 55:45 to 51:49, 55:45 to 50:50, 54:46 to 53:47, 54:46 to 52:48, 54:46 to 51:49, 54:46 to 50:50, 53:47 to 52:48, 53:47 to 51:49, 53:47 to 50:50, 52:48 to 51:49, 52:48 to 50:50 or 51:49 to 50:50. In specific embodiments, the saponin matrix composition includes an effective amount of the antigen and Matrix-C particles, but not Matrix-A particles.

The present invention also provides a method for eliciting cross-presentation of an antigen in a subject in need thereof. The method includes administering to the subject an effective amount of a saponin matrix composition, thereby eliciting cross-presentation of the antigen in the subject. The saponin matrix composition includes an effective amount of the antigen and Matrix-C particles, but not Matrix-A particles. In certain embodiments, such Matrix-C-only compositions are used to selectively enhance antigen cross-presentation and CD8 T-cell responses while controlling cytotoxicity, as further described herein.

Across numerous embodiments, the invention described herein independently provides multiple, interrelated advantages over existing saponin-based adjuvants and vaccine adjuvant systems. In particular, the specified saponin matrix compositions including Matrix-A particles and Matrix-C particles at defined fixed weight ratios in the range of 70:30 to 10:90 of Matrix-A particles to Matrix-C particles deliver an unexpectedly favorable balance of potent antigen-cross-presentation and controlled cytotoxicity, resulting in markedly enhanced antigen-specific CD8+ T-cell responses compared to conventional Matrix-M formulations employing an 85:15 Matrix-A to Matrix-C ratio. The compositions permit administration of doses sufficient to elicit robust activation of antigen-presenting cells and induction of IFN-γ-producing CD8+ T cells while reducing excessive cytotoxicity associated with Matrix-C particles, thereby widening the functional therapeutic window relative to known Matrix-M adjuvant systems. By merging, in a single composition, the cross-presentation-inducing capacity of Matrix-A particles (despite their lack of fatty acyl chains) with the strong adjuvant potency but higher inherent cytotoxicity of Matrix-C particles, the invention provides a synergistic functional profile that could not be predicted from the behavior of either particle type alone or from the previously standardized Matrix-M ratio. These saponin matrix compositions confer further practical and clinical benefits. They can be used to enhance both the magnitude and quality of adaptive immune responses, including robust CD4+ and CD8+ T-cell responses, in the context of diverse antigens such as viral antigens and tumor antigens, thus enabling applications in prophylactic and therapeutic vaccines, including cancer immunotherapy. Because the compositions are based on well-characterized, reproducibly manufactured Matrix-A and Matrix-C particles built from defined Quillaja saponaria saponin fractions, they maintain the desirable physical stability and formulation compatibility of Matrix-based systems while providing superior functional performance, including improved antigen cross-presentation and enhanced induction of antigen-specific CD8+ T cells relative to standard Matrix-M adjuvant. These functional improvements are achieved without requiring changes to the fundamental manufacturing platform for the individual Matrix particles, facilitating integration into existing production, quality control, and regulatory frameworks, and thereby offering a commercially attractive path to next-generation adjuvant products with demonstrable performance advantages.

The invention also offers significant strategic value for vaccine developers and commercial partners. By enabling stronger CD8+ T-cell responses from protein-based or subunit vaccines that otherwise elicit weak or absent cytotoxic T-cell responses, the disclosed saponin matrix compositions can support dose-sparing strategies, broaden indications, and potentially improve durability and breadth of protective immunity against viral infections and cancer. In some embodiments, the ability to modulate cross-presentation and cytotoxicity through controlled adjustment of the Matrix-A to Matrix-C ratio within the disclosed range provides a tunable platform for tailoring immune profiles to different disease targets, patient populations, and combination regimens, including with other immunomodulators. Collectively, these scientific, clinical, and manufacturing advantages provide a strong basis for regulatory differentiation and for commercial licensing, positioning the disclosed compositions as high-value improvements over existing adjuvants and as enabling technologies for a wide range of current and future vaccine products.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1, with reference to TABLE 1, shows a structural representation of the most common Quillaja saponins in saponin Fraction-A and saponin Fraction-C. Abbreviations: Ac=acetyl, Api=apiose, Ara=arabinose, C=carbon, Fuc=fucose, Gal=galactose, Glc=glucose, GlcA=glucuronic acid, H=hydrogen, O=oxygen, Rn=residue (index n), Rha=rhamnose, Xyl=xylose; v.v.=vice versa.

FIG. 2 shows general chemical structures of saponin Fraction-A and saponin Fraction-C. In certain embodiments, saponin Fraction-A and saponin Fraction-C are defined by their quantitative saponin compositions as determined by quantitative ¹H, ¹³C-HSQC-NMR spectroscopy. Signals corresponding to characteristic molecular moieties known in the literature are quantified. Aglycones and substituents R¹ to R⁵ are described in TABLE 2. Abbreviations: Qa=Quillaic acid; QaOH=Quillaic acid 22β-OH; Gyp=Gypsogenin; PAOAc=23-O—Ac Phytolaccagenic acid; Fuc=Fucose; Gal=Galactose; Ara=Arabinose; Rha=Rhamnose; Xyl=Xylose; Fa=Fatty acyl; OAc=O-Acetyl.

FIGS. 3A-3C illustrate that Matrix-M accumulates inside lysosomes. Bone marrow-derived dendritic cells (BMDCs) loaded with LysoTracker Red DND-99 were treated with BODIPY-labeled Matrix-M, Matrix-A, or Matrix-C. (3A) Representative images acquired by live-cell spinning-disk confocal imaging show individual channels (LysoTracker, BODIPY), overlays, and a DIC (Differential Interference Contrast) channel captured after each treatment (scalebar=10 μm). (3B) The fluorescence intensity kinetics of BODIPY fluorescence for the indicated Matrix concentrations are displayed. (3C) Pearson's correlation coefficients (PCC) for BODIPY and LysoTracker fluorescence over time are shown. Data shown in 3B and 3C represent smoothed averages from at least three technical and biological replicates±SEM.

FIGS. 4A-4C illustrate how Matrix-M induces lysosomal membrane permeabilization (LMP). BMDCs were treated with either Matrix-M, Matrix-A, or Matrix-C (for 1 h), bafilomycin (BafA1, for 1 h), or L-Leucyl-L-Leucine methyl ester (LLOMe, for 15 min) at the indicated concentrations followed by staining with LysoTracker Deep Red and FVS520 for 15 and 7 min, respectively. Stained cells were analyzed by flow cytometry. (4A) Flow cytometry histograms showing LysoTracker fluorescence intensity in viable BMDCs from representative samples. (4B) Normalized geometric mean fluorescence intensity (gMFI) of LysoTracker Deep Red in BMDCs. Data were normalized to untreated control samples and are shown as the mean±SD; pooled from three independent experiments. Statistical differences between untreated control and treated samples were determined by one-way ANOVA followed by Dunnett's multiple comparisons test (****p<0.0001). (4C) Images of LysoTracker-stained BMDCs before and after treatment with BODIPY-labeled Matrix-M, as described in FIGS. 3A-3C. The scalebar=10 μm.

FIGS. 5A-5B show that Matrix-induced LMP results in the release of lysosomal contents into the cytosol. BMDCs were preloaded overnight with dextran particles of different sizes and fluorochromes (Alexa Fluor 488-conjugated 3-kDa dextran and tetramethylrhodamine-conjugated 40-kDa dextran). The following day, cells were pretreated with bafilomycin A1 (BafA1, 30 nM) or left untreated for 1 hour. Live-cell spinning-disk confocal imaging was performed at 3 min intervals for up to 5 h, before and after adding Matrix-M, Matrix-A (both at 10 μg/ml), or Matrix-C (1 μg/ml), to monitor lysosomal release of dextran particles into the cytosol. (5A) Representative images showing individual channels (3- and 40-kDa dextrans) and overlays at the indicated time points and treatment conditions (scalebar=20 μm). (5B) The percentage of cells with intact lysosomes over time for each treatment condition is shown. Data are pooled from three independent experiments and presented as the percentage of cells with intact lysosomes±95% confidence interval.

FIGS. 6A-6G illustrate how Matrix-M induces NLRP3 inflammasome activation and IL-1β release in vitro. BMDCs were either non-primed (6A) or LPS-primed (6A-6G) and treated with increasing doses of Matrix-M, Matrix-A, or Matrix-C for 5 hours. Released IL-1β levels were measured in the supernatants by ELISA. Before treatment with Matrix adjuvants, cells were pre-treated for 1 h with no inhibitor, V-ATPase inhibitor bafilomycin A1 (BafA1) (6B), cathepsin B inhibitor CA074-Me (6C), NLRP3-related inhibitors MCC950 (6D) and Ac-YVAD-cmk (6F), or gasdermin D pore formation inhibitor disulfiram (6G) at the indicated concentrations. Additionally, IL-1β levels were measured in NLRP3-deficient and wild type (WT, C57BL/6) BMDCs after Matrix adjuvant treatment (6E). Results are shown as the mean±SD from at least three independent experiments.

FIGS. 7A-7D illustrates that Matrix-M-induced IL-18 release is NLRP3 inflammasome independent. BMDCs were either non-primed (7A-7D) or LPS-primed (7A) treated with increasing doses of Matrix-M, Matrix-A, or Matrix-C for 24 hours. Released IL-18 levels were measured by ELISA. Before treatment with Matrix adjuvants, cells were pre-treated for 1 h with V-ATPase inhibitor bafilomycin A1 (BafA1) (7B) or NLRP3-assembly inhibitor MCC950 (7C) at the indicated concentration. Similarly, IL-18 levels were measured in NLRP3-deficient and wild type (WT, C57BL/6) BMDCs after Matrix adjuvant treatment (7D). Results are shown as mean±SD of three independent experiments.

FIGS. 8A-8E shows that NLRP3 inflammasome activation is not required for Matrix-M adjuvanticity in vivo. C57BL/6 (WT) and Nlrp3 KO mice (n=10/group) were immunized with 0.1 μg SARS-CoV-2 rS (rS) alone or adjuvanted with 5 μg Matrix-M, Matrix-A, or Matrix-C. Serum samples obtained 13, 20, and 28 days after the primary immunization were evaluated by ELISA for IgG1 (8A) and IgG2c (8B) antibody titers against rS protein and for hACE2 receptor-inhibiting antibody titers (8C). At day 28, splenocytes from individual mice were isolated and the number of cells producing IFN-γ, IL-2, or IL-4 in response to rS protein restimulation was determined by FluoroSpot assay (8D). Individual titers (ELISA) or spot forming units (SPU)/10⁶ splenocytes are shown as individual symbols, with horizontal bars representing geometric mean values, and error bars represent their 95% confidence intervals. Additionally, splenocytes were analyzed by flow cytometry (intracellular staining) to assess frequencies of rS-specific CD4⁺ and CD8⁺ T cells producing IFN-γ and IL-4 (8E). Data for each individual mouse response are shown with symbols and group means are represented by horizontal bars. The data (8A-8E) were analyzed for differences between WT and and Nlrp3 KO mice within each adjuvanted group by one-way ANOVA with Šidák's multiple comparisons test (*for p<0.05, **for p<0.01).

FIGS. 9A-9G illustrates that Matrix-M enables antigen cross-presentation in BMDCs. BMDCs were loaded with LysoTracker Red DND-99, washed and imaged using spinning-disk confocal microscopy for 30 minutes. Following the initial pre-bleaching image acquisition, cells were treated with Alexa Fluor 647-labeled rS (0.5 or 1 μg/mL) and BODIPY-Matrix-M (1 or 2.5 μg/mL) and live-cell imaging continued for an additional 120 min. Pearson's correlation coefficients (PCC) for the channel combination LysoTracker and Alexa Fluor 647 (9A), and BODIPY and Alexa Fluor 647 (9B) were determined as indicators of lysosome-antigen or Matrix-M-antigen colocalization, respectively. Data in panels 9A and 9B are presented as smoothed averages ±SEM, pooled from at least three experiments, each performed in triplicates. (9C-9G) BMDCs were exposed to 0.8 mg/mL OVA and indicated concentrations of Matrix-M, Matrix-A, or Matrix-C for 24 hours. Cells were analyzed by flow cytometry for surface markers and for H-2 kB bound to SIINFEKL complexes, with the latter indicative of antigen cross-presentation. The percentage of viable BMDCs positive for H-2Kb/SIINFEKL complexes is shown as representative contour plots (9C) and corresponding graphs (9D) for untreated and Matrix-treated groups. (9E) Representative flow cytometry plots and (9F) graphs show the proportion of major subpopulations within BMDC culture, identified as non-DC-like cells (CD11c⁻ MHCII⁻, dark blue), immature DC-like cells (CD11c⁺ MHCII^{-lo}, turquoise) and mature DC-like cells (CD11c⁺ MHCII^{hi}, gray). (9G) The percentage of various BMDC subpopulations within cells positive for H-2Kb/SIINFEKL complexes is shown. Data in panels 9C-9E are representative of three independent experiments, while graphs in 9F and 9G show pooled data from three independent experiments, presented as the mean±SD.

FIGS. 10A-10F show that lysosomal acidification and proteasomal degradation contribute to Matrix-induced antigen cross-presentation. BMDCs were exposed to 80 μg/mL OVA and indicated concentrations of Matrix-M, Matrix-A, or Matrix-C for 5 h (10A, 10C, 10E). In control experiments, 5 ng/mL SIINFEKL instead of OVA was added (10B, 10D, 10F). Cells were co-cultured for 18 h with B3Z cells, which produce β-galactosidase upon TCR recognition of SIINFEKL presented on H-2 kB. BMDCs were pretreated for 1 h with bafilomycin A1 (BafA1) to inhibit lysosomal acidification (10C, 10D) or with epoxomicin (10E, 10F) to inhibit proteasomal activity. Results shown are representative of two independent experiments, with data presented as mean values of triplicates±SD.

FIGS. 11A-11F illustrate that Matrix-M generates dose dependent CD8⁺ T-cell responses in vivo. C57BL/6 mice were immunized with OVA alone or adjuvanted with 5 μg Matrix-M. Pools of DCs isolated from the dLNs were co-cultured for 3 days with naïve CD8⁺ OT-I T cells. Representative histograms for CellTrace Violet (CTV) dilution are shown (11A); these were used to calculate the percentage of divided cells by FlowJo proliferation analysis (11B). Surface expression (median fluorescence intensity; MFI) of the T-cell activation marker CD44 within CD8⁺ T cells was analyzed, as displayed in the representative contour plot and corresponding graph (11C, 11D). Each symbol represents one individual pool, with the solid horizontal bars representing group means±SD. Unpaired t-test was applied for comparisons between the two groups (11B, 11D). (11E, 11F) BALB/c mice (n=10/group) were immunized with 0.1 μg SARS-CoV-2-rS alone or adjuvanted with the indicated doses of Matrix-M (11E), Matrix-A, or Matrix-C (11F). Splenocytes from individual mice were isolated to determine the frequency of CD8⁺ T cells that produce IFN-γ after rS protein stimulation. Each symbol represents an individual mouse; horizontal bars represent group means. One-way ANOVA with Tukey's multiple comparisons test was applied to compare the groups to antigen only (p-values * for p<0.05, ** for p<0.01, *** for p<0.001, **** for p<0.0001).

FIG. 12 shows Matrix adjuvant-lysosome colocalization analysis. Spinning-disk confocal microscopy (live-cell imaging) was applied to study the localization of Matrix adjuvant in lysosomes. LysoTracker-loaded BMDCs were incubated with BODIPY-Matrix-M, Matrix-A, or Matrix-C and imaged for 120 min every 3 minutes. Pearson's correlation coefficient (PCC) for the channel combination BODIPY and LysoTracker was calculated as a measure of the colocalization of Matrix adjuvants with lysosomes. Graphs show the PCC for the respective Matrix adjuvant type and concentration at selected time points. Mean values from at least three replicates are shown.

FIG. 13 displays the LMP flow cytometry gating strategy. Lysosomal membrane permeabilization (LMP) in BMDCs after Matrix adjuvant treatment was assessed using a flow cytometry-based assay. A representative gating strategy was used for the flow cytometric analysis to measure LysoTracker Deep Red fluorescence intensity in viable (FVS520 negative) single cells. Gating for doublet cell exclusion was done but is not shown.

FIGS. 14A-14D shows viability and IL-1β secretion of Matrix-treated BMDCs. LPS-primed BMDCs were treated with increasing doses of Matrix-M, Matrix-A, or Matrix-C for 5 h. Cell viability was assessed by WST-1 assay (14A, 14C, 14D) and IL-1β levels in supernatants were measured by ELISA (14B). Before treatment with Matrix adjuvants, cells were pre-treated for 1 h with no inhibitor, V-ATPase inhibitor bafilomycin A1 (14A), gasdermin D pore formation inhibitors necrosulfonamide (14B, 14D), or with disulfiram (14C) at the indicated concentrations. Results are shown as the mean±SD from three experiments.

FIG. 15 displays the gating strategy for BMDC characterization. A gating strategy for BMDCs after 7 days of culture with GM-CSF. The upper panel shows exclusion of cell debris and doublets, further gated for live cells as FVS-780 negative. Live cells were gated as non-DC-like cells (red) that are CD11c− and MHCII−, as immature DC-like (blue) that are CD11c⁺ and negative or low for MHCII, or as mature DC-like cells (orange) that are MHCII high. Histograms show the indicated markers in the three DC subpopulations (red, blue, and orange) and the respective FMO control (gray).

FIGS. 16A-16H show BMDC activation after Matrix-M, Matrix-A, or Matrix-C treatment. BMDCs were treated for 24 h with the indicated concentrations of Matrix-M, Matrix-A, or Matrix-C and were analyzed by flow cytometry. Representative histograms show the MHCII and CD86 expression levels of live cells for each Matrix treatment at varying concentrations (16A, 16E) and for 1 μg/ml Matrix-M, Matrix-A, and Matrix-C (16B, 16F). Untreated controls are shown as shaded gray. Summary data showing MHCII and CD86 expression levels in BMDCs treated with Matrix-M, Matrix-A, or Matrix-C are presented in panels 16C and 16G. Data represent mean±SD from three independent experiments. Two-way ANOVA with Tukey's multiple comparisons test was applied for comparison (p-values * for p<0.05, ** for p<0.01, *** for p<0.001, **** for p<0.0001). For comparison with LPS (10 ng/ml), a representative histogram shows MHCII (16D) and CD86 (16H) expression levels on live BMDCs.

FIGS. 17A-17B shows phenotype and activation of NLRP3-deficient BMDCs. Untreated BMDCs generated from wild type (WT) and Nlrp3 KO mice were analyzed by flow cytometry for their phenotype (see also FIG. 15: non-DC-like, immature DC-like and mature DC-like cells) (17A) and for their activation based on MHCII and CD86 expression levels (17B).

FIGS. 18A-18C displays the ex vivo analysis of dendritic cells (DCs) from immunized mice. C57BL/6 mice were immunized with 10 μg OVA alone or adjuvanted with 5 μg Matrix-M. Iliac lymph node samples were collected 24 h post immunization, processed and sorted for CD11c⁺ DCs cells. DCs used for T-cell co-culture (in FIGS. 11A-11D) were analyzed by flow cytometry with the following gating strategy (18A): The upper panel shows exclusion of cell debris and doublets and identification of antigen-presenting cells (CD11c⁺ MHCII⁺) after excluding lymphocytes (DUMP; CD19⁺/CD3⁺ for B and T cells, respectively). Antigen-presenting cells were further classified into CD88⁺ or CD88⁻ subsets. Within the CD88-subset, CD26⁺ cells were identified as cDCs, with CD64⁻ cDCs subdivided into cDC1 (XCR1⁺) and cDC2 (CD172a⁺), while cDCs that were CD64⁺ were classified as inflammatory cDC2, CD88⁺ cells expressing CD64 were defined as monocyte-derived DCs (moDCs). The frequencies of these DC subsets from pooled samples (n=4-5) are presented in (18B), while the MFI of CD86 within CD11c⁺ MHCII⁺ DCs is shown in (C). Each symbol represents one individual pool, bar graphs represent mean values, and error bars represent the standard deviation (mean±SD). Statistical analyses were performed using One-way ANOVA with Bonferroni's multiple comparisons test (B) or an unpaired t-test (C) (*** for p<0.001, **** for p<0.0001).

FIGS. 19A-19-D show that the immune response is dependent on Matrix-M dose. BALB/c mice (n=10/group) were immunized with 0.1 μg SARS-CoV-2 rS alone or adjuvanted with the indicated doses of Matrix-M. Serum samples obtained at day 28 (after primary immunization) were evaluated by ELISA for IgG1 and IgG2a (19A) antibody titers against rS protein and for hACE2 receptor-inhibiting antibody titers (19B). Splenocytes from individual mice were isolated at day 28 and analyzed for the number of cells producing IFN-γ, IL-2, or IL-4 in response to SARS-CoV-2 rS protein restimulation (19C). Each symbol represents individual mice, and horizontal bars represent geometric mean values. Additionally, splenocytes from individual mice were analyzed by flow cytometry (intracellular staining) for the frequency of CD4+ T cells that are CD44-high and produce IFN-γ, TNF, or IL-4 after rS protein re-stimulation (19D). Each individual mouse response is shown with each symbol; the solid horizontal bars represent the group mean. The data (19A-19D) were analyzed by one-way ANOVA with Tukey's/Dunnett's multiple comparisons test (* for p<0.05, ** for p<0.01, *** for p<0.001, **** for p<0.0001). No line indicates a significant difference compared to antigen only, and a line between two groups indicates a significant difference between adjuvanted vaccine groups.

FIGS. 20A-20E illustrate generation of antigen-specific IFN-γ responses with immunizations using different ratios of Matrix-A to Matrix-C. C57BL/6 mice were immunized with 1 μg rS protein only or adjuvanted with 5 μg Matrix with different ratios of Matrix-A to Matrix-C ranging from 100% Matrix-A to 100% Matrix-C. Mice received a booster dose after 1 week and analysis was performed 2 weeks after primary immunization. Splenocytes were restimulated with a rS protein (20A, 20C, 20E) or short S CD8 epitope peptide (20B, 20D) that is only recognized by CD8+ T cells. IFN-γ (20A, 20B), IL-2 (20C, 20D) and IL-4 (20E) responses were detected using Fluorospot. Mean and 95% CI are displayed together with individual data. The data were analyzed by one-way ANOVA with Tukey's multiple comparisons test (* for p<0.05, ** for p<0.01, *** for p<0.001, for p<0.0001).

FIGS. 21A-21D illustrate generation of antigen-specific IFN-γ CD8+ T-cell responses (A,B) and CD4+ T cell responses (21C, 21D) following immunizations with different ratios of Matrix-A to Matrix-C. C57BL/6 mice were immunized with 1 μg rS protein only or adjuvanted with 5 μg Matrix with different ratios of Matrix-A to Matrix-C ranging from 100% Matrix-A to 100% Matrix-C. Mice received a booster dose after 1 week and analysis was performed 2 weeks after primary immunization. Splenocytes were restimulated with a rS protein (21A, 21C) or short S CD8 epitope peptide (21B, 21D) that only is recognized by CD8+ T cells. IFN-γ responses were detected by intracellular cytokine staining. Mean and standard deviation are displayed together with individual data. The data were analyzed by one-way ANOVA with Tukey's multiple comparisons test (* for p<0.05, ** for p<0.01, *** for p<0.001, **** for p<0.0001).

FIG. 22 illustrates generation of antigen-specific CD4+ T cell IL-4 responses following immunizations with different ratios of Matrix-A to Matrix-C. C57BL/6 mice were immunized with 1 μg rS protein only or adjuvanted with 5 μg Matrix with different ratios of Matrix-A to Matrix-C ranging from 100% Matrix-A to 100% Matrix-C. Mice received a booster dose after 1 week and analysis was performed 2 weeks after primary immunization. Splenocytes were restimulated with a rS protein. IL-4 responses were detected by intracellular cytokine staining. Mean and standard deviation are displayed together with individual data. The data were analyzed by one-way ANOVA with Tukey's multiple comparisons test (* for p<0.05, ** for p<0.01, *** for p<0.001, **** for p<0.0001).

FIGS. 23A-23B illustrate generation of antigen-specific serum responses following immunizations using different ratios of Matrix-A to Matrix-C. C57BL/6 mice were immunized with 1 μg rS protein only or adjuvanted with 5 μg Matrix with different ratios of Matrix-A to Matrix-C ranging from 100% Matrix-A to 100% Matrix-C. Mice received a booster dose after 1 week and analysis was performed 2 weeks after primary immunization. Serum samples obtained at day 12 (after primary immunization) were evaluated by ELISA for IgG1 (23A) and IgG2c (23B) antibody titers against rS protein. Geometric mean and 95% CI are displayed together with individual data. The data were log-transformed before analysis by one-way ANOVA with Tukey's multiple comparisons test (**** for p<0.0001).

### DEFINITIONS

As used herein, the following words, phrases, and symbols are generally intended to have the meanings as set forth below, unless a different meaning is apparent from the context.

The term “saponin matrix composition” refers to a composition that comprises a saponin component formulated as Matrix-A particles, Matrix-C particles, or a combination thereof at a defined or definable weight ratio, optionally together with one or more additional excipients and/or antigens, and that is suitable for administration to a subject to modulate an immune response.

The term “Matrix-M adjuvant” refers to a saponin matrix composition that comprises Matrix-A particles and Matrix-C particles at a fixed weight ratio of about 85:15 of Matrix-A particles to Matrix-C particles, typically formulated with cholesterol and phosphatidylcholine as cage-like particles in an aqueous buffer suitable for administration, and functionally serving as an adjuvant; such a composition is generally used herein as a reference prior-art composition for comparative purposes.

The term “Matrix-A particles” refers to particulate structures formed from saponin Fraction-A of Quillaja saponaria Molina saponins together with one or more lipids (for example, cholesterol and phosphatidylcholine), typically having a cage-like morphology and a nanometer-scale size distribution, and being characterized by relatively low cytotoxicity and a capacity to activate antigen-presenting cells and induce antigen cross-presentation.

The term “Matrix-C particles” refers to particulate structures formed from saponin Fraction-C of Quillaja saponaria Molina saponins together with one or more lipids (for example, cholesterol and phosphatidylcholine), typically having a cage-like morphology and a nanometer-scale size distribution, and being characterized by potent adjuvant activity but relatively higher cytotoxicity compared to Matrix-A particles.

The term “fixed weight ratio” refers to a predetermined ratio of the weight of a first component to the weight of a second component, for example the weight of Matrix-A particles to the weight of Matrix-C particles, that is maintained within specified tolerance limits during manufacture and in the final composition, and that does not substantially change during normal storage and handling, such that functional comparisons between compositions can be made under comparable dosing and administration conditions.

The term “total weight of the Matrix-A particles and the Matrix-C particles” refers to the combined weight contribution of Matrix-A particles and Matrix-C particles present in a saponin matrix composition, excluding the weight contribution of other formulation components such as antigens, buffers, additional excipients, or diluents.

The term “aqueous composition” refers to a composition in which water is the predominant solvent and in which the saponin matrix (for example, Matrix-A particles, Matrix-C particles, or combinations thereof) are dispersed, optionally together with one or more additional components that are dissolved such as antigens, buffers, salts, stabilizers, or preservatives.

The term “lyophilized composition” refers to a composition that has been subjected to a freeze-drying process to remove water and yield a solid or semi-solid material comprising the saponin matrix and optionally one or more additional components (such as an antigen), which material is configured to be reconstituted with an aqueous diluent prior to administration.

The term “spray-dried composition” refers to a composition obtained by atomizing a liquid feed comprising the saponin matrix and optionally one or more additional components into a drying chamber, thereby removing solvent and forming solid or semi-solid particles suitable for reconstitution or direct administration, including in dry powder or inhalation formats.

The term “solid composition” refers to a non-liquid formulation state in which the saponin matrix is present as a powder, granulate, pellet, tablet, or other solid or semi-solid form, optionally in combination with excipients and/or antigens, and which is suitable for storage, reconstitution, or administration.

The term “Fraction-A” refers to a saponin fraction obtained from Quillaja saponaria Molina extracts, enriched for saponins that elute in a defined window (for example, around 39% acetonitrile) under specified reversed-phase chromatographic conditions, and that corresponds to the material used to generate Matrix-A particles.

The term “Fraction-C” refers to a saponin fraction obtained from Quillaja saponaria Molina extracts, enriched for saponins that elute in a defined window (for example, around 49% acetonitrile) under specified reversed-phase chromatographic conditions, and that corresponds to the material used to generate Matrix-C particles.

The term “highly purified Quillaja extract” refers to an extract of Quillaja saponaria Molina saponins characterized by a saponin content of at least about 90% by weight and defined ranges of specific saponin species (for example, QS-A and QS-C) and water content, suitable as a starting material for preparing Fraction-A and Fraction-C.

The term “cage-like structures” refers to particulate assemblies of saponins and lipids, such as Matrix particles, that adopt a closed or quasi-closed morphology enclosing an interior space, typically with a mean size on the order of tens of nanometers as determined by suitable analytical methods.

The term “nanometer-scale size distribution” refers to a particle size distribution in which the majority of particles have an effective diameter in the nanometer range, for example from about 10 nm to about 200 nm, as measured by dynamic light scattering, electron microscopy, or other suitable techniques.

The term “antigen” refers to any molecule or molecular structure that can be specifically recognized by the immune system of a subject, for example by antibodies and/or T-cell receptors, and includes, without limitation, proteins, peptides, polysaccharides, lipids, conjugates thereof, and mimetics thereof, that are capable of eliciting or boosting an antigen-specific immune response when administered to a subject.

The term “viral antigen” refers to an antigen that is derived from, based on, or structurally mimics one or more components of a virus, including whole viral proteins, protein subunits, fragments, peptides, or virus-like particles, and that is capable of eliciting or boosting an immune response directed against the corresponding virus in a subject.

The term “tumor antigen” refers to an antigen that is expressed by tumor cells, overexpressed by tumor cells relative to normal cells, mutated in tumor cells, or otherwise associated with tumor biology, including tumor-associated antigens and tumor neoantigens, and that is capable of eliciting or boosting an immune response directed against tumor cells in a subject.

The term “tumor-associated antigen (TAA)” refers to a tumor antigen that is present on tumor cells and also present, typically at lower levels, on certain normal cells or tissues, and that can serve as a target for tumor-directed immune responses when administered as part of a vaccine or immunotherapeutic composition.

The term “tumor neoantigen” refers to a tumor antigen that arises from a somatic mutation, gene rearrangement, or other tumor-specific alteration, and that is not present in the normal, non-tumor proteome of the subject, and that is capable of eliciting or boosting tumor-specific immune responses.

The term “SARS-CoV-2 S protein antigen” refers to an antigen that comprises, consists essentially of, or consists of all or a portion of the spike(S) protein of severe acute respiratory syndrome coronavirus 2 (SARS-CoV-2), including native, modified, stabilized, truncated, or variant forms thereof, and that is capable of eliciting or boosting an immune response against SARS-CoV-2.

The term “PIV3 antigen” refers to an antigen that comprises, consists essentially of, or consists of all or a portion of one or more proteins of human parainfluenza virus type 3 (PIV3), including, for example, fusion (F) or hemagglutinin-neuraminidase (HN) proteins or fragments thereof, and that is capable of eliciting or boosting an immune response against PIV3.

The term “RSV F0 antigen” refers to an antigen that comprises, consists essentially of, or consists of all or a portion of the fusion protein F (including F0 precursor or its subunits) of respiratory syncytial virus (RSV), including native, prefusion-stabilized, or modified variants thereof, and that is capable of eliciting or boosting an immune response against RSV.

The term “hMPV F antigen” refers to an antigen that comprises, consists essentially of, or consists of all or a portion of the fusion protein F of human metapneumovirus (hMPV), including native or modified forms, and that is capable of eliciting or boosting an immune response against hMPV.

The term “adjuvant” refers to a component that, when administered with an antigen or as part of an immunogenic composition, increases or modulates the magnitude, quality, or duration of the immune response to the antigen in a subject, without necessarily being itself the primary target of that immune response and without necessarily being immunogenic on its own.

The term “protective immunity” refers to an immune status in a subject resulting from immunization or prior exposure that reduces, to any detectable degree, the likelihood, severity, or duration of disease or infection associated with a given pathogen or cancer, including partial or complete protection, where even a minor reduction is considered protective.

The term “antigen cross-presentation” refers to the process by which antigen-presenting cells internalize exogenous antigen, process it, and present peptides derived from the antigen on major histocompatibility complex class I (MHC I) molecules, thereby enabling activation of CD8 T cells specific for that antigen.

The term “cross-presentation of an antigen” refers to antigen cross-presentation as defined herein, with respect to a particular antigen of interest.

The term “antigen-presenting cell” refers to any cell capable of processing and presenting antigen-derived peptides in the context of major histocompatibility complex molecules to T cells, including, without limitation, dendritic cells, macrophages, B cells, and monocyte-derived antigen-presenting cells.

The term “antigen presenting cells” refers to a population of cells that comprises one or more types of antigen-presenting cells as defined herein.

The term “activation of antigen-presenting cells” refers to a change in phenotype or function of antigen-presenting cells indicative of an activated state, including, for example, increased expression of costimulatory molecules (such as CD86 or CD80), increased expression of MHC molecules, production of cytokines, enhanced antigen uptake, processing, or presentation, or combinations thereof.

The term “antigen-specific CD8 T cells” refers to CD8-positive T lymphocytes that recognize a particular antigen via their T-cell receptor and that can be detected by assays such as peptide-MHC multimer binding, intracellular cytokine staining, or functional cytotoxicity assays.

The term “antigen-specific CD4 T cells” refers to CD4-positive T lymphocytes that recognize a particular antigen via their T-cell receptor and that can be detected by assays such as peptide-MHC multimer binding, intracellular cytokine staining, proliferation assays, or cytokine secretion assays.

The term “antigen-specific CD8 T-cell responses” refers to immune responses that involve activation, expansion, or functional activity of antigen-specific CD8 T cells, as evidenced by one or more measurable parameters such as cell frequency, cytokine production, proliferation, or cytotoxic activity.

The term “increased production of IFN-γ by antigen-specific CD8 T cells” refers to a higher amount or frequency of interferon-γ production by antigen-specific CD8 T cells in one condition relative to a comparator condition, as measured by an appropriate assay (for example, intracellular cytokine staining, ELISPOT, FluoroSpot, or cytokine quantification in culture supernatants).

The term “cytotoxic CD8 T-cell responses” refers to antigen-specific CD8 T-cell responses that mediate killing or functional impairment of target cells presenting antigen-derived peptides, as evidenced by one or more assays such as target cell lysis, granzyme or perforin expression, or related readouts.

The term “cytokine-producing CD8 T cells” refers to CD8 T cells that produce one or more cytokines, such as IFN-γ, TNF, or IL-2, upon stimulation with antigen, as detected by assays including intracellular cytokine staining or cytokine secretion measurement.

The term “increased antigen cross-presentation” refers to a higher level or frequency of antigen cross-presentation in one condition relative to a comparator condition, as evidenced by increased presentation of antigen-derived peptides on MHC I molecules or increased activation of antigen-specific CD8 T cells in functional assays.

The term “increased activation of antigen-presenting cells” refers to a higher level or frequency of activation of antigen-presenting cells in one condition relative to a comparator condition, as evidenced by one or more markers or functions associated with activation, such as increased surface expression of MHC or costimulatory molecules, cytokine production, or enhanced antigen processing.

The term “cellular immune responses” refers to immune responses that predominantly involve T cells, NK cells, or other immune cells mediating cell-based effector functions, including, for example, antigen-specific CD4 and CD8 T-cell responses, in contrast to purely humoral antibody responses.

The term “humoral immune responses” refers to immune responses that predominantly involve antibodies produced by B cells or plasma cells, including antigen-specific immunoglobulin responses, neutralizing antibody responses, and related serological outcomes.

The term “NLRP3 inflammasome activation” refers to the activation of a multiprotein complex containing NLRP3 that results in caspase-1 activation and processing of pro-inflammatory cytokines such as IL-1β and IL-18, as evidenced by increased cytokine release or other suitable readouts.

The term “IL-1β release” refers to the secretion of interleukin-1β from cells into the extracellular environment under defined conditions, as measured by assays such as ELISA or cytokine bead arrays.

The term “IL-18 release” refers to the secretion of interleukin-18 from cells into the extracellular environment under defined conditions, as measured by assays such as ELISA or cytokine bead arrays.

The term “dose-dependent CD8+ T-cell responses” refers to CD8+ T-cell responses whose magnitude varies as a function of the administered dose of the Matrix adjuvant, as evidenced by increased or decreased frequencies or functional readouts across different dose levels.

The term “composition having a weight ratio of 85:15 of the Matrix-A particles to the Matrix-C particles” refers to a saponin matrix composition in which the weight of Matrix-A particles to the weight of Matrix-C particles is about 85:15, typically with allowed variation consistent with manufacturing tolerances and the definition of “about” provided herein, and that serves as a reference composition for comparative performance assessments when evaluating increases in antigen cross-presentation, activation of antigen-presenting cells, or antigen-specific CD8 T-cell responses delivered by the disclosed Matrix-A:Matrix-C ratios.

The term “under comparable dosing and administration conditions” refers to conditions in which compositions being compared are administered to subjects using substantially the same dose of antigen, substantially the same dose (or dose range) of saponin matrix, and substantially the same route, schedule, and regimen of administration, such that observed differences in immune responses can reasonably be attributed to differences in composition rather than gross differences in dosing or route.

The term “increased frequency” refers to a higher proportion of a defined cell population (for example, antigen-specific CD8 T cells, activated antigen-presenting cells) in one condition relative to a comparator condition, as measured by an appropriate assay such as flow cytometry, where the increase can be expressed as an absolute or percentage change.

The term “increased proportion” refers to an increased fraction or percentage of a defined population of cells or responses in one condition relative to a comparator condition, as determined by an appropriate analytical method.

The term “at least about 25%/30%/50%/75%/100% increase” refers to an increase in a measured parameter (such as frequency of antigen-specific CD8 T cells or level of cytokine production) that is equal to or greater than the specified percentage relative to a comparator condition (for example, a composition having a weight ratio of 85:15 of Matrix-A particles to Matrix-C particles under comparable dosing and administration conditions), with ‘about’ allowing for experimental variability as further defined herein and the parameter measured by an appropriate assay such as flow cytometry, ELISA, ELISPOT, or FluoroSpot.

The term “lysosomal membrane permeabilization (LMP)” refers to a process in which the integrity of the lysosomal membrane is compromised, resulting in altered retention or release of lysosomal contents, and which can be detected by changes in lysosomal tracer dyes, release of lysosomally loaded markers into the cytosol, or related readouts.

The term “increased lysosomal membrane permeabilization” refers to a higher degree or frequency of lysosomal membrane permeabilization in one condition relative to a comparator condition, as measured by one or more suitable assays such as loss of LysoTracker fluorescence or cytosolic appearance of lysosomal markers.

The term “loss of LysoTracker fluorescence intensity” refers to a reduction in fluorescence signal from LysoTracker or similar lysosomotropic dyes in cells treated under a given condition relative to a comparator condition, interpreted as indicative of reduced lysosomal integrity, altered lysosomal pH, or lysosomal content release.

The term “dextran release into the cytosol” refers to the appearance or increased presence of fluorescently labeled dextran molecules, previously localized in lysosomes, within the cytosolic compartment of a cell, as detected by imaging or other analytical methods, and is indicative of lysosomal membrane permeabilization.

The term “cytosolic fluorescence of lysosomally loaded dextran” refers to the measurable fluorescence signal of labeled dextran molecules in the cytosol of cells that were previously loaded with dextran in lysosomes, serving as an indicator of lysosomal content leakage into the cytosol.

The term “disrupted lysosomes” refers to lysosomes that have undergone lysosomal membrane permeabilization to an extent that they no longer retain their contents or normal dye accumulation characteristics, as evidenced by imaging or other assays.

The term “intact lysosomes” refers to lysosomes that retain their structural integrity and functional characteristics, including retention of lysosomotropic dyes and encapsulated markers, in contrast to disrupted lysosomes.

The term “dosage” refers to the quantity or amount of a component (for example, antigen, Matrix-A particles, Matrix-C particles, or total saponin matrix) administered to a subject per administration event, per unit time, per body weight, per body surface area, or per vaccination regimen, as specified in the context of use.

The term “dosage of Matrix-A particles and Matrix-C particles” refers to the combined amount of Matrix-A particles and Matrix-C particles administered to a subject in a given dose or dosing regimen, expressed, for example, in micrograms, milligrams, or another suitable unit.

The term “dosage of Matrix-C particles” refers to the amount of Matrix-C particles administered to a subject in a given dose or dosing regimen, independent of the amount of Matrix-A particles, and may be expressed in weight units or weight per body weight units.

The term “fixed weight ratio of antigen to Matrix-A and Matrix-C particles” refers to a predetermined ratio of the total weight of antigen(s) to the total weight of Matrix-A and Matrix-C particles in a composition, which ratio is maintained within specified tolerance limits during manufacture and in the final composition.

The term “fixed weight ratio of antigen to Matrix-C particles” refers to a predetermined ratio of the total weight of antigen(s) to the weight of Matrix-C particles in a composition, which ratio is maintained within specified tolerance limits during manufacture and in the final composition.

The term “co-formulated” refers to a state in which an antigen and a saponin matrix composition (for example, Matrix-A and Matrix-C particles) are present together in the same dosage form, container, or preparation at the time of administration, as opposed to being administered from separate containers or at substantially different times.

The term “administered at the same anatomical site within a time window of up to 24 hours” refers to administration of two or more components such that they are delivered to substantially the same physical location in the body (for example, the same muscle or adjacent region) and the administrations occur within a period not exceeding about 24 hours between the first and last administration.

The term “parenteral administration” refers to administration of a composition by a route that bypasses the gastrointestinal tract, including, without limitation, intramuscular, subcutaneous, intradermal, intravenous, intraarterial, or intraperitoneal administration.

The term “intramuscular injection” refers to parenteral administration in which a composition is injected into muscle tissue of a subject using a syringe, needle, or other suitable delivery device.

The term “subcutaneous injection” refers to parenteral administration in which a composition is injected into the subcutaneous tissue of a subject using a syringe, needle, or other suitable delivery device.

The term “intradermal injection” refers to parenteral administration in which a composition is injected into the dermal layer of the skin of a subject using a suitable delivery device.

The term “pulmonary administration” refers to administration of a composition to the respiratory tract, typically via inhalation, such that the composition contacts the lungs and/or lower airways, including by nebulization, aerosolization, or dry powder inhalation.

The term “intranasal administration” refers to administration of a composition into the nasal cavity, typically via sprays, drops, aerosols, or powders, such that the composition contacts the nasal mucosa.

The term “dry powder formulation” refers to a solid particulate formulation suitable for inhalation or intranasal delivery that comprises the saponin matrix and optionally one or more antigens and excipients, and that is free of or substantially free of liquid solvent at the time of administration.

The term “subject in need thereof” refers to any animal, including a human, that would benefit from the administration of the composition or method described, including for treatment, prevention, prophylaxis, amelioration of disease or symptoms, or induction or boosting of an immune response.

The term “treatment for cancer” refers to any intervention that results in a therapeutic benefit with respect to cancer in a subject, including, without limitation, reducing tumor burden, slowing or halting tumor growth, preventing metastasis, prolonging survival, improving quality of life, or preventing or delaying recurrence.

The term “treating the cancer in the subject” refers to administering a composition or carrying out a method that achieves treatment for cancer as defined herein in the subject.

The term “inducing adaptive immune response against a viral pathogen” refers to eliciting or boosting an antigen-specific immune response involving B cells and/or T cells that is directed against one or more antigens of a viral pathogen, as evidenced by measurable antibody and/or T-cell responses.

The term “prophylactic use” refers to use of a composition or method in a subject who is at risk of developing a disease or condition but who does not yet exhibit clinical signs, with the goal of preventing, delaying, or reducing the likelihood or severity of the disease or condition.

The term “therapeutic use” refers to use of a composition or method in a subject who already has a disease or condition, with the goal of treating, ameliorating, or managing the disease or condition, including reducing symptoms, slowing progression, or improving survival.

The term “H-2Kb/SIINFEKL complexes” refers to complexes of the murine MHC class I molecule H-2Kb presenting the SIINFEKL peptide, used as a model readout of antigen cross-presentation by antigen-presenting cells.

The term “FluoroSpot assay” refers to a fluorescence-based immunospot assay used to detect and quantify single cells secreting specific cytokines (for example, IFN-γ, IL-2, IL-4) upon antigen restimulation.

The term “intracellular cytokine staining” refers to a flow cytometry method in which cells are fixed, permeabilized, and stained with labeled antibodies to detect cytokines produced within the cells after stimulation.

The term “about” refers to a value, range, or endpoint that may vary within experimental or manufacturing tolerances without materially changing the basic function or effect of the value, range, or endpoint, and generally encompasses variations of up to ±10% or other contextually appropriate ranges around a stated value unless otherwise specified.

The term “comprising” is intended to be open-ended and refers to the inclusion of one or more recited elements or features without excluding the presence of additional, unrecited elements or features.

The term “including” refers to the inclusion of one or more recited elements or features and, unless otherwise specified, is used in a non-limiting sense similar to “comprising,” such that additional, unrecited elements or features can also be present.

The term “such as” is used to introduce examples that are illustrative and not limiting, and the examples following “such as” do not exclude other, unlisted examples that fall within the scope of the broader concept.

The term “effective amount” refers to an amount of a component (for example, antigen or saponin matrix composition) that, when administered to a subject, produces a desired biological or clinical effect, such as induction or enhancement of an immune response, reduction of disease severity, or achievement of prophylactic or therapeutic benefit, and may vary depending on factors including the subject's condition, route of administration, and concurrent therapies.

### DETAILED DESCRIPTION OF THE INVENTION

The inventors have discovered that Matrix-A particles exhibit a higher capacity to promote activation of antigen-presenting cells than Matrix-C particles and, unexpectedly, are themselves capable of inducing antigen cross-presentation despite being formed from saponins that lack a fatty acyl chain. In view of these findings, the present invention provides saponin matrix compositions that include Matrix-A particles and Matrix-C particles at defined fixed weight ratios in the range of 70:30 to 10:90 of Matrix-A particles to Matrix-C particles. These compositions can be formulated with a wide variety of antigens, such as viral antigens or tumor antigens, and administered to subjects in need thereof to elicit potent cellular and humoral immune responses, including enhanced frequencies of antigen-specific CD8+ T cells that produce IFN-γ and other effector cytokines, relative to compositions comprising a Matrix-M adjuvant having an 85:15 Matrix-A to Matrix-C ratio.

In certain embodiments, the saponin matrix compositions are administered together with a protein or subunit antigen that would otherwise induce little or no antigen-specific CD8+ T-cell response, in order to drive robust cross-presentation and cytotoxic T-cell priming while avoiding the excessive cytotoxicity that can be associated with higher proportions of Matrix-C particles. Without wishing to be bound by theory, the enhanced functional profile of these compositions is believed to result from a favorable balance between the strong APC-activating and cross-presentation-inducing activity of Matrix-A particles and the potent but more cytotoxic activity of Matrix-C particles, tuned by the selected Matrix-A:Matrix-C weight ratio. In specific implementations, administration of these compositions in mice adjuvanted with SARS-CoV-2 rS protein at the disclosed ratios yields at least a 30%, specifically at least 50%, more specifically at least 75%, and more specifically at least 100% increase in frequencies of antigen-specific CD8+ T cells producing IFN-γ compared to a composition having a Matrix-A:Matrix-C weight ratio of 85:15 under comparable dosing and administration conditions, demonstrating that the compositions provide an unexpectedly potent synergistic effect on CD8+ T-cell immunity.

The invention further encompasses vaccine compositions that include the disclosed saponin matrix compositions together with one or more antigens, pharmaceutical dosage forms and regimens for human and veterinary administration, and methods of using such compositions to induce or enhance protective immunity. Illustrative methods include inducing production of IFN-γ by antigen-specific CD8+ T cells in a subject, eliciting cross-presentation of an antigen by antigen-presenting cells, treating cancer using tumor-associated antigens or tumor neoantigens, and inducing adaptive immune responses against viral pathogens such as SARS-CoV-2, PIV3, RSV, or hMPV. The Matrix-A and Matrix-C particles employed in these compositions can be prepared from well-characterized Fraction-A and Fraction-C saponin fractions of Quillaja saponaria Molina using scalable reversed-phase liquid chromatography processes and formulated with cholesterol and phosphatidylcholine into stable cage-like particles, allowing the present invention to be implemented on existing Matrix manufacturing platforms while delivering materially improved immunogenic performance.

### Compositions and Methods for Modulating Immune Response

The present invention relates to saponin matrix compositions comprising defined fixed weight ratios of Matrix-A particles to Matrix-C particles, vaccine compositions incorporating such saponin matrix compositions together with antigens, and methods of using these compositions to elicit potent adaptive immune responses—in particular, enhanced antigen-specific CD8⁺ T-cell responses—in subjects in need thereof. The invention provides a material advance over existing saponin-based adjuvant systems, including conventional Matrix-M adjuvant (which comprises Matrix-A particles and Matrix-C particles at a fixed weight ratio of 85:15), by demonstrating that shifting the Matrix-A:Matrix-C ratio into the range of about 70:30 to 10:90 (e.g., 60:40, 59:41, 58:42, 57:43, 56:44, 55:45, 54:46, 53:47, 52:48, 51:49, or 50:50) unexpectedly and substantially increases antigen cross-presentation, antigen-presenting cell activation, and the frequency of antigen-specific CD8⁺ T cells producing IFN-γ and other effector cytokines.

The invention encompasses, without limitation: (i) saponin matrix compositions; (ii) vaccine compositions comprising an antigen and a saponin matrix composition; (iii) methods for inducing production of IFN-γ by antigen-specific CD8⁺ T cells; (iv) methods for eliciting cross-presentation of an antigen; (v) methods for treating cancer using tumor antigens or tumor neoantigens; (vi) methods for inducing adaptive immune responses against viral pathogens; (vii) methods for activating antigen-presenting cells, and (viii) methods for inducing an adaptive immune response that is substantially independent of NLRP3 inflammasome activation. The compositions and methods are applicable across diverse therapeutic and prophylactic contexts, including infectious disease vaccines and cancer immunotherapy, and are suitable for use in human and veterinary subjects.

### The Matrix-A and Matrix-C Particles

Matrix-A particles and Matrix-C particles are approximately 40 nm-sized, stable cage-like structures formed by separately mixing saponin Fraction-A and saponin Fraction-C, respectively, of Quillaja saponaria Molina saponins with cholesterol and phosphatidylcholine in the presence of the detergent Mega-10, followed by removal of Mega-10 by diafiltration. The resulting particles are formulated in a phosphate buffer solution comprising approximately 137 mM sodium chloride, 2.7 mM potassium chloride, and 9.8 mM phosphate at pH 7.2, yielding physically stable preparations suitable for combination with a variety of antigens.

Matrix-A particles are formed from saponin Fraction-A, which comprises saponins that lack fatty acyl chains and elute at approximately 39% acetonitrile under reversed-phase high-performance liquid chromatography conditions. Matrix-A particles are characterized by relatively low cytotoxicity and a robust capacity to activate antigen-presenting cells and promote antigen cross-presentation. Matrix-C particles are formed from saponin Fraction-C, which comprises saponins possessing fatty acyl chains and elute at approximately 49% acetonitrile under the same conditions. Matrix-C particles are characterized by potent adjuvant activity but comparatively higher cytotoxicity relative to Matrix-A particles.

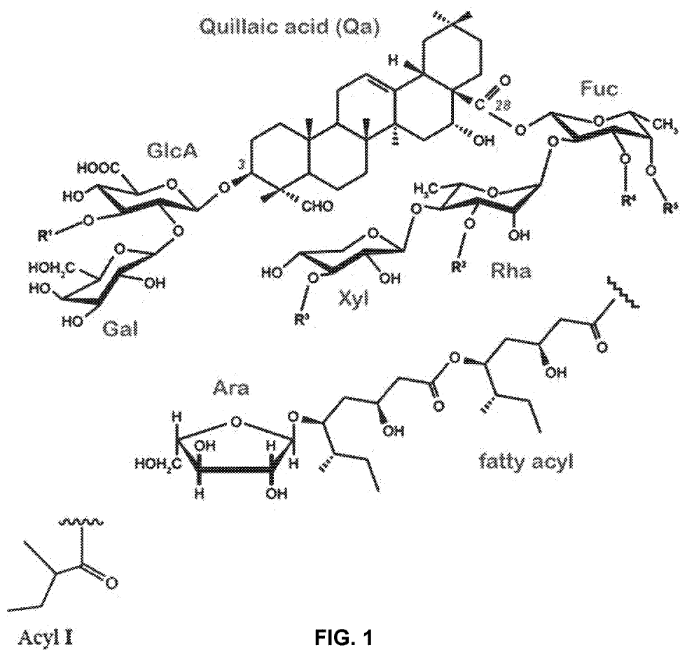

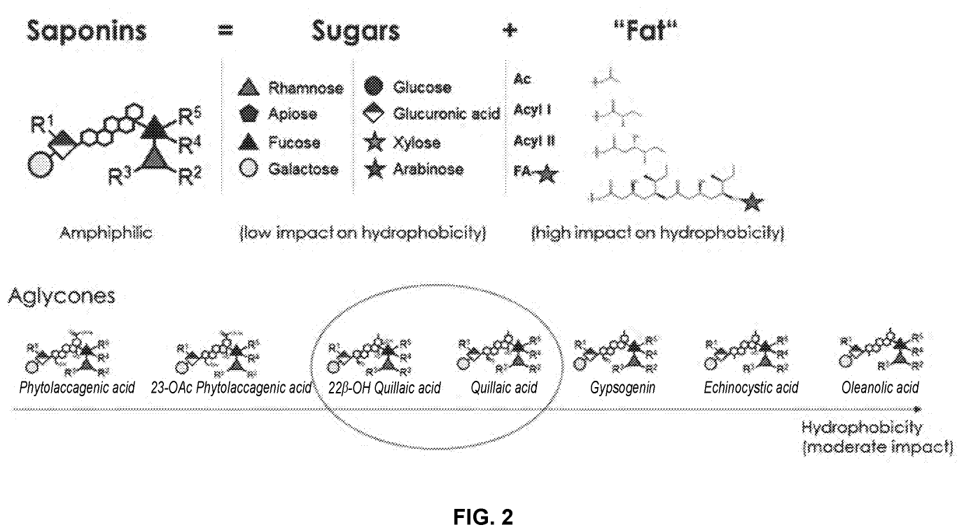

The structural features of the most common Quillaja saponins in Fraction-A and Fraction-C are summarized in TABLE 2 and illustrated in FIG. 1 and FIG. 2. The key structural distinction between the two fractions relates to the presence or absence of fatty acyl substituents at the R⁴ and R⁵ positions of the saponin aglycone.

TABLE 2
Structural representation of most common Quillaja saponins
in saponin Fraction-A and saponin Fraction-C.

| Saponin Fraction | R¹ | R² | R³ | R⁴ | R⁵ |
|---|---|---|---|---|---|
| Fraction-A | Rha | H | H/Api/Xyl | Rha | Ac or acyl I |
| | | Glc | Api | | |
| | Xyl | H | H/Api/Xyl | | |
| | | Glc | H/Api | | |
| Fraction-C | Rha | H | Xyl/Api | H and | |
| | | | | fatty acyl | |
| | | | | (or v.v.) | |
| | Xyl | | | Ac | fatty acyl |
| | | | H and | | |
| | | | fatty acyl | | |
| | | | (or v.v.) | | |
| | | | H | | |

Abbreviations: Ac = acetyl, Api = apiose, Ara = arabinose, C = carbon, Fuc = fucoses, Gal = galactose, Glc = glucose, GlcA = glucuronic acid, H = hydrogen, O = oxygen, R^{a} = residue (index n), Rha = rhamnose, Xyl = xylose; v.v. = vice versa.

### ABBREVIATIONS

### Preparation of Fraction-A and Fraction-C

The Matrix-A and Matrix-C particles can be produced from saponin Fraction-A and saponin Fraction-C, respectively, which are prepared from an extract of Quillaja saponaria Molina saponins using reversed-phase liquid chromatography. In specific embodiments, the starting material is a highly purified Quillaja extract having a saponin content of ≥90% (weight/weight), a saponin QS-A content of 13.0±6.0%, a saponin QS-C content of 21.0±9.0%, and a water content of ≤8.0%. Such a highly purified Quillaja extract is available, for example, from Desert King (United States). The process comprises: (a) loading the extract onto a reversed-phase liquid chromatography stationary phase; and (b) eluting with a reversed-phase liquid chromatography mobile phase to obtain saponin Fraction-A and/or saponin Fraction-C. The Matrix-A particles and Matrix-C particles are then separately manufactured from the respective fractions as described above. Other suitable approaches to producing Matrix-A and Matrix-C particles are also encompassed by the invention.

### The Saponin Matrix Compositions: Fixed Matrix-A:Matrix-C Ratio

The key inventive discovery is that a defined fixed weight ratio of Matrix-A particles to Matrix-C particles in the range of 70:30 to 10:90 provides a functionally superior adjuvant profile relative to conventional Matrix-M adjuvant (85:15 Matrix-A:Matrix-C). Specific ratios within the range include, without limitation, about 60:40, 59:41, 59:41, 58:42, 57:43, 56:44, 55:45, 54:46, 53:47, 52:48, 51:49, and 50:50. In further specific sub-ranges, the fixed weight ratio is from about 57:43 to about 53:47, or from about 59:41 to about 51:49. In specific embodiments, the ratio of the Matrix-A particles to the Matrix-C particles is about: 60:40 to 59:41, 60:40 to 58:42, 60:40 to 57:43, 60:40 to 56:44, 60:40 to 55:45, 60:40 to 54:46, 60:40 to 53:47, 60:40 to 52:48, 60:40 to 51:49, 60:40 to 50:50, 59:41 to 58:42, 59:41 to 57:43, 59:41 to 56:44, 59:41 to 55:45, 59:41 to 54:46, 59:41 to 53:47, 59:41 to 52:48, 59:41 to 51:49, 59:41 to 50:50, 58:42 to 57:43, 58:42 to 56:44, 58:42 to 55:45, 58:42 to 54:46, 58:42 to 53:47, 58:42 to 52:48, 58:42 to 51:49, 58:42 to 50:50, 57:43 to 56:44, 57:43 to 55:45, 57:43 to 54:46, 57:43 to 53:47, 57:43 to 52:48, 57:43 to 51:49, 57:43 to 50:50, 56:44 to 55:45, 56:44 to 54:46, 56:44 to 53:47, 56:44 to 52:48, 56:44 to 51:49, 56:44 to 50:50, 55:45 to 54:46, 55:45 to 53:47, 55:45 to 52:48, 55:45 to 51:49, 55:45 to 50:50, 54:46 to 53:47, 54:46 to 52:48, 54:46 to 51:49, 54:46 to 50:50, 53:47 to 52:48, 53:47 to 51:49, 53:47 to 50:50, 52:48 to 51:49, 52:48 to 50:50 or 51:49 to 50:50.

The central rationale for these compositions is that they effectively merge the potent antigen cross-presentation and APC-activating properties of Matrix-A particles with the strong adjuvant potency of Matrix-C particles, while avoiding the excessive cytotoxicity associated with higher proportions of Matrix-C particles. The compositions thereby provide an unexpectedly favorable and synergistic balance of adjuvant potency, antigen cross-presentation capacity, and tolerability that could not be predicted from the behavior of either particle type alone, or from the known performance of conventional 85:15 Matrix-M adjuvant.

### Immunological Performance Relative to Conventional Matrix-M

When the saponin matrix composition is administered together with an antigen to a subject, it induces an increased production of IFN-γ by antigen-specific CD8⁺ T cells compared to a saponin matrix composition comprising Matrix-A and Matrix-C particles at a weight ratio of 85:15. In specific embodiments, the increase in IFN-γ production by antigen-specific CD8⁺ T cells is at least 30%, specifically at least 50%, more specifically at least 75%, and more specifically at least 100%, relative to the 85:15 comparator.

In further specific embodiments, the composition-when used as an adjuvant for immunization of a mouse with a SARS-CoV-2 recombinant spike (rS) protein antigen-leads to an elevated frequency of antigen-specific CD8⁺ T-cell IFN-γ responses as detected ex vivo by restimulation of splenocytes from the immunized mouse with the antigen, compared to a saponin matrix composition with a weight ratio of 85:15 of Matrix-A particles to Matrix-C particles. The frequency is elevated by at least 30%, specifically at least 50%, more specifically at least 75%, and more specifically at least 100%.

Additionally, the compositions at the disclosed ratios induce, relative to the 85:15 comparator:
Increased antigen cross-presentation by antigen-presenting cells, including an increased proportion of antigen-presenting cells positive for MHC class I-peptide complexes (e.g., H-2K^{b}/SIINFEKL in murine models), by at least about 25%;
Increased activation of antigen-presenting cells, as evidenced by upregulated surface expression of MHCII and CD86;
Increased induction of lysosomal membrane permeabilization (LMP) in antigen-presenting cells, as evidenced by reductions in LysoTracker fluorescence intensity and increased cytosolic appearance of lysosomal dextran markers;
Increased frequencies of antigen-specific CD8⁺ T cells producing IFN-γ and TNF (polyfunctional CD8⁺ T cells); and
Increased antigen-specific IL-2 responses detectable by FluoroSpot.

These results collectively demonstrate that the disclosed saponin matrix compositions provide a superior CD8⁺ T-cell adjuvant platform relative to conventional Matrix-M, without requiring modification to the underlying manufacturing processes for the individual Matrix-A and Matrix-C particles.

### Vaccine Compositions

The invention further provides vaccine compositions comprising (i) an antigen and (ii) a saponin matrix composition as described above. The antigen and the saponin matrix composition may be co-formulated in the same dosage unit, or may be provided as separate components for co-administration (e.g., administered at the same anatomical site within a time window of up to 24 hours).

### Antigen:Matrix Dosing Ratios

In specific embodiments, the antigen is present in the vaccine composition at a fixed weight ratio of the antigen to the total weight of the Matrix-A and Matrix-C particles in the range of about 0.1 μg:200 μg to about 400 μg:20 μg of antigen to total Matrix-A plus Matrix-C particles. In further specific embodiments, the antigen is present at a fixed weight ratio of about 5 μg:100 μg to about 400 μg:50 μg of antigen to total Matrix-A plus Matrix-C particles. In additional embodiments, the antigen may be present at a fixed weight ratio of the antigen to Matrix-C particles alone in the range of about 0.1 μg:200 μg to about 400 μg:20 μg. Illustrative specific ratios include about 10 μg antigen to 50 μg total Matrix, about 50 μg antigen to 50 μg total Matrix, about 100 μg antigen to 50 μg total Matrix, and about 400 μg antigen to 50 μg Matrix-C. For cancer vaccine applications, a specific antigen dose is about 300 μg to about 400 μg of tumor peptide antigen per administration.

### Viral and Tumor Antigens

The antigen in the vaccine compositions may be a viral antigen or a tumor antigen. One or more (e.g., 1, 2, 3, 4, etc.) antigens may be included in the composition.

Viral antigens include, without limitation, a SARS-CoV-2 S protein antigen (including native, modified, stabilized, or truncated forms of the spike protein), a PIV3 antigen, an RSV F0 antigen, and an hMPV F antigen. These antigens are suitable for prophylactic and therapeutic vaccines against the corresponding viral pathogens.

Tumor antigens include tumor-associated antigens (TAAs) and tumor neoantigens. TAAs are non-mutated antigens overexpressed in tumor tissue. Tumor neoantigens are antigens arising from somatic mutations or gene rearrangements specific to tumor cells, and are not present in the normal proteome of the subject. The tumor antigen may be selected from, without limitation: Human Papillomavirus (HPV E6 or E7), Melanoma-Associated Antigen (MAGE), Prostate-Specific Antigen (PSA), Kirsten rat sarcoma Glycine 12 Aspartic Acid (KRAS G12D), Kirsten rat sarcoma Glycine 12 Valine (KRAS G12V), Kirsten rat sarcoma Glycine 12 Cysteine (KRAS G12C), Kirsten rat sarcoma Glycine 12 Alanine (KRAS G12A), Kirsten rat sarcoma Glycine 13 Aspartic Acid (KRAS G13D), V-raf Murine Sarcoma Viral Oncogene homolog B1 Valine 600 Glutamic Acid (BRAF V600E), Phosphatidylinositol-4,5-bisphosphate 3-kinase catalytic subunit alpha H1047R (PIK3CA H1047R), TP53 R175H, TP53 R273H, SYT-SSX1, SYT-SSX2, PAX3-FOX01, Ewing Sarcoma Breakpoint Region 1-Friend Leukemia Integration 1 (EWS-FLI1), Breakpoint Cluster Region-Abelson Murine Leukemia Viral Oncogene Homolog 1 (ABL1) (BCR-ABL (b3a2, b2a2)), Coiled-Coil Domain Containing 6-Rearranged during Translocation (CCDC6-RET), Fibroblast Growth Factor Receptor 3-Transforming Acidic Coiled-Coil containing protein 3 (FGFR3-TACC3), Tropomyosin 3-Anaplastic Lymphoma Kinase (TPM3-ALK), Tropomyosin 4-Anaplastic Lymphoma Kinase (TPM4-ALK), Neurotrophic Tyrosine Kinase Receptor Type (NTRK1) fusions (Tropomyosin 3-Neurotrophic Receptor Tyrosine Kinase 1 (TPM3-NTRK1), ETS Variant Transcription Factor 6-Neurotrophic Tyrosine Receptor Kinase 3 (ETV6-NTRK3)), DEK-AFF2, Transforming Growth Factor Beta Receptor 2 (TGFBR2) frameshift (-1A), Activin A receptor type 2A (ACVR2A) frameshift (-1A), Caspase 5 (CASP5) frameshift, TGF-Beta Activated Kinase 1 Binding Protein 1 (TAB1F) frameshift, and Asteroid Structure-Specific Endonuclease 1 (ASTE1)/HT001 frameshift.

### Formulation and Physical Forms

The saponin matrix composition and vaccine compositions may be provided in a range of physical forms suitable for diverse manufacturing and administration scenarios.

In specific embodiments, the compositions are provided as aqueous compositions, wherein the saponin matrix is dispersed in phosphate-buffered saline (approximately 137 mM sodium chloride, 2.7 mM potassium chloride, and 9.8 mM phosphate, pH 7.2). This formulation provides physical stability and compatibility with parenteral administration and combination with various antigens.

In additional embodiments, the aqueous composition may be subjected to spray drying or lyophilization to yield a solid composition in powder, granulate, or other solid form. Spray-dried compositions are particularly suited for use as dry powder formulations for pulmonary or intranasal administration without prior reconstitution, for example using a dry powder inhaler. Lyophilized compositions may similarly be formulated as dry powders for inhalation or intranasal use, or may be reconstituted with a pharmaceutically acceptable liquid vehicle to provide a liquid dosage form suitable for parenteral, oral, or inhalation administration, including aerosol formulations. The solid composition may also be formulated into conventional solid dosage forms, including tablets and capsules.

It is to be understood that these formulation forms are illustrative rather than limiting; the invention encompasses all pharmaceutically acceptable formulation states in which the defined Matrix-A:Matrix-C weight ratio is maintained.

### Methods of Use

The invention encompasses a broad range of methods employing the saponin matrix compositions and vaccine compositions described herein. The unifying feature of all methods is administration of a saponin matrix composition comprising Matrix-A particles and Matrix-C particles, typically at a fixed weight ratio in the range of 70:30 to 10:90, to a subject in need thereof, optionally together with an antigen.

### Inducing IFN-γ Production by Antigen-Specific CD8⁺ T Cells

The invention provides methods for inducing production of IFN-γ by antigen-specific CD8⁺ T cells in a subject in need thereof, comprising administering (i) a saponin matrix composition as described herein and (ii) an effective amount of an antigen to the subject. In specific embodiments, the method results in an increase in IFN-γ production by antigen-specific CD8⁺ T cells of at least 30%, specifically at least 50%, more specifically at least 75%, and more specifically at least 100%, compared to administration of a saponin matrix composition having an 85:15 Matrix-A:Matrix-C weight ratio. This method is applicable to both prophylactic immunization (e.g., against viral pathogens) and therapeutic settings (e.g., cancer immunotherapy), where robust CD8⁺ T-cell responses are critical for efficacy.

### Eliciting Cross-Presentation of an Antigen

The invention provides methods for eliciting cross-presentation of an antigen in a subject in need thereof, comprising administering (i) a saponin matrix composition as described herein and (ii) an effective amount of the antigen to the subject, thereby eliciting cross-presentation of the antigen in the subject. Cross-presentation refers to the process by which antigen-presenting cells (APCs) internalize exogenous antigen, process it, and present derived peptides on MHC class I molecules, enabling activation of antigen-specific CD8⁺ T cells.

In specific embodiments, the cross-presentation of the antigen is increased compared to a method employing a saponin matrix composition with an 85:15 Matrix-A:Matrix-C weight ratio. The increase is at least 30%, specifically at least 50%, more specifically at least 75%, and more specifically at least 100%.

In further specific embodiments, the cross-presentation in the subject involves:
Increased activation of antigen-presenting cells and enhanced CD8⁺ T-cell priming, relative to the 85:15 comparator, by at least 30%, specifically at least 50%, more specifically at least 75%, and more specifically at least 100%; and
Increased induction of lysosomal membrane permeabilization (LMP) in antigen-presenting cells, relative to the 85:15 comparator, by at least 30%, specifically at least 50%, more specifically at least 75%, and more specifically at least 100%. LMP is mechanistically linked to the escape of antigen from the lysosomal compartment into the cytosol, enabling proteasomal processing and MHC class I loading, and is a key mechanistic feature by which the disclosed compositions promote cross-presentation.

The invention also encompasses cross-presentation methods employing compositions comprising Matrix-C particles in the absence of Matrix-A particles, where cross-presentation is elicited by the antigen-incorporating or cytosolic escape-promoting activity of Matrix-C particles.

### Treating Cancer

The invention provides methods of treatment for cancer in a subject in need thereof, comprising administering a tumor antigen (including a tumor neoantigen) together with a saponin matrix composition as described herein to the subject. The disclosed compositions are particularly suited for cancer immunotherapy because a robust CD8⁺ T-cell response is required for the efficacy of therapeutic cancer vaccines, which direct the immune system to recognize and destroy tumor cells.

The tumor neoantigen in these methods may be any peptide containing a tumor-specific somatic mutation capable of being presented by MHC molecules. In specific embodiments, the tumor neoantigen is selected from: KRAS G12D, KRAS G12V, KRAS G12C, KRAS G12A, KRAS G13D, BRAF V600E, PIK3CA H1047R, TP53 R175H, TP53R273H, SYT-SSX1, SYT-SSX2, PAX3-FOX01, EWS-FLI1, BCR-ABL (b3a2, b2a2), CCDC6-RET, FGFR3-TACC3, TPM3-ALK, TPM4-ALK, NTRK1 fusions (TPM3-NTRK1, ETV6-NTRK3), DEK-AFF2, TGFBR2 frameshift (-1A), ACVR2A frameshift (-1A), CASP5 frameshift, TABIF frameshift, ASTE1 frameshift, and HT001 frameshift. In additional embodiments, the tumor antigen may be a TAA such as HPV E6 or E7, MAGE, or PSA.

### Inducing Adaptive Immune Responses Against Viral Pathogens

The invention provides methods of inducing an adaptive immune response against a viral pathogen in a subject in need thereof, comprising administering a viral antigen together with a saponin matrix composition as described herein to the subject. In specific embodiments, the viral antigen is a SARS-CoV-2 S protein antigen, a PIV3 antigen, an RSV F0 antigen, or an hMPV F antigen. The method may be for inducing protective immunity against the virus from which the viral antigen is derived, including SARS-CoV-2, PIV3, RSV, and hMPV.

These methods leverage the ability of the disclosed saponin matrix compositions to drive both robust humoral (antibody) immune responses and potent cellular (CD4⁺ and CD8+ T-cell) immune responses, providing a comprehensive immune profile suited to protection against viral infection and clearance of virus-infected cells.

### Activating Antigen-Presenting Cells

The invention provides methods for activating antigen-presenting cells (APCs) in a subject in need thereof, comprising administering a saponin matrix composition as described herein to the subject, thereby activating APCs in the subject. APCs encompassed by these methods include, without limitation, dendritic cells, macrophages, and B cells. In specific embodiments, the activation of APCs—as evidenced by increased surface expression of MHCII and CD86 and increased antigen processing capacity—is greater compared to a method employing a saponin matrix composition with an 85:15 Matrix-A:Matrix-C weight ratio, with an increase of at least 30%, specifically at least 50%, more specifically at least 75%, and more specifically at least 100%.

### Compositions for Use and Use for Manufacture of a Medicament

The invention also encompasses the saponin matrix compositions and vaccine compositions described herein for use in the therapeutic and prophylactic methods disclosed above, as well as use of such compositions for the manufacture of a medicament for any of the disclosed medical uses, thereby providing coverage under applicable statutory frameworks for method-of-treatment, composition-for-use, and Swiss-style use claims.

### Administration

### Route of Administration

In specific embodiments, the saponin matrix composition or vaccine composition is administered parenterally, including but not limited to intramuscular injection, subcutaneous injection, or intradermal injection. In additional embodiments, the composition may be administered by pulmonary inhalation (e.g., using a dry powder inhaler or nebulizer), by intranasal administration (e.g., as a spray, aerosol, or dry powder), or by oral administration following reconstitution of a lyophilized or spray-dried composition in a pharmaceutically acceptable liquid vehicle.

### Dosage

In specific embodiments, the saponin matrix composition is administered at a dosage of the total Matrix-A particles plus Matrix-C particles of about 20 μg to about 200 μg per administration, with a specific range of about 50 μg to about 100 μg. In alternative embodiments, dosage may be expressed as Matrix-C particles alone: about 20 μg to about 200 μg per administration, specifically about 50 μg to about 100 μg. Illustrative specific dosages include about 20 μg, 50 μg, 75 μg, 100 μg, 150 μg, and 200 μg of total Matrix-A plus Matrix-C, or of Matrix-C alone, per administration. Dosage ranges are applicable to both human and veterinary subjects, and may be adjusted as clinically appropriate.

### Subject

The subject in these methods can include a human, cow, horse, pig, or sheep, with humans being a specific subject. The compositions and methods are applicable to both prophylactic use (in subjects at risk of infection or cancer who do not yet exhibit clinical signs) and therapeutic use (in subjects who already have an infection or cancer).

### Embodiments

The embodiments describing the subject matter below are for illustration purposes only, and do not otherwise limit the scope of the disclosed subject matter, as defined by the claims.

### Fixed Matrix-A:Matrix-C Ratio Window

In specific embodiments, the saponin matrix composition includes Matrix-A particles and Matrix-C particles, wherein the Matrix-A particles and the Matrix-C particles are present at a fixed weight ratio in the range of about 70:30 to 10:90 of the Matrix-A particles to the Matrix-C particles.

In specific embodiments, the saponin matrix composition includes Matrix-A particles and Matrix-C particles, wherein the Matrix-A particles and the Matrix-C particles are present at a fixed weight ratio in the range of about 57:43 to about 53:47 of the Matrix-A particles to the Matrix-C particles.

In specific embodiments, the saponin matrix composition includes Matrix-A particles and Matrix-C particles, wherein the Matrix-A particles and the Matrix-C particles are present at a fixed weight ratio in the range of about 60:40 to about 53:47 of the Matrix-A particles to the Matrix-C particles.

In specific embodiments, the saponin matrix composition includes Matrix-A particles and Matrix-C particles, wherein the Matrix-A particles and the Matrix-C particles are present at a fixed weight ratio in the range of about 57:43 to about 50:50 of the Matrix-A particles to the Matrix-C particles.

In specific embodiments, the saponin matrix composition includes Matrix-A particles and Matrix-C particles, wherein the Matrix-A particles and the Matrix-C particles are present at a fixed weight ratio of about 52:48 of the Matrix-A particles to the Matrix-C particles.

In specific embodiments, the saponin matrix composition includes Matrix-A particles and Matrix-C particles, wherein the Matrix-A particles and the Matrix-C particles are present at a fixed weight ratio of about 58:42 of the Matrix-A particles to the Matrix-C particles.

In specific embodiments, the saponin matrix composition includes Matrix-A particles and Matrix-C particles, wherein the Matrix-A particles and the Matrix-C particles are present at a fixed weight ratio of about 54:46 of the Matrix-A particles to the Matrix-C particles.

In specific embodiments, the saponin matrix composition includes Matrix-A particles and Matrix-C particles, wherein the Matrix-A particles and the Matrix-C particles are present at a fixed weight ratio of about 56:44 of the Matrix-A particles to the Matrix-C particles.

In specific embodiments, the saponin matrix composition includes Matrix-A particles and Matrix-C particles, wherein the Matrix-A particles and the Matrix-C particles are present at a fixed weight ratio in the range of about 52:48 to about 50:50 of the Matrix-A particles to the Matrix-C particles.

In specific embodiments, the saponin matrix composition includes Matrix-A particles and Matrix-C particles, wherein the Matrix-A particles and the Matrix-C particles are present at a fixed weight ratio in the range of about 60:40 to about 58:42 of the Matrix-A particles to the Matrix-C particles.

In specific embodiments, the saponin matrix composition includes Matrix-A particles and Matrix-C particles, wherein the Matrix-A particles and the Matrix-C particles are present at a fixed weight ratio in the range of about 54:46 to about 52:48 of the Matrix-A particles to the Matrix-C particles.

In specific embodiments, the saponin matrix composition includes Matrix-A particles and Matrix-C particles, wherein the Matrix-A particles and the Matrix-C particles are present at a fixed weight ratio in the range of about 59:41 to about 51:49 of the Matrix-A particles to the Matrix-C particles.

In specific embodiments, the saponin matrix composition includes Matrix-A particles and Matrix-C particles, wherein the Matrix-A particles and the Matrix-C particles are present at a fixed weight ratio of about 51:49 of the Matrix-A particles to the Matrix-C particles.

In specific embodiments, the saponin matrix composition includes Matrix-A particles and Matrix-C particles, wherein the Matrix-A particles and the Matrix-C particles are present at a fixed weight ratio of about 59:41 of the Matrix-A particles to the Matrix-C particles.

In specific embodiments, the saponin matrix composition includes Matrix-A particles and Matrix-C particles, wherein the Matrix-A particles and the Matrix-C particles are present at a fixed weight ratio of about: 60:40, 59:41, 58:42, 57:43, 56:44, 55:45, 54:46, 53:47, 52:48, 51:49, or 50:60 of the Matrix-A particles to the Matrix-C particles.

### Relative Performance Vs 85:15 Matrix-M Benchmark

In specific embodiments, the saponin matrix composition includes Matrix-A particles and Matrix-C particles, wherein the Matrix-A particles and the Matrix-C particles are present at a fixed weight ratio in the range of about 70:30 to 10:90 of the Matrix-A particles to the Matrix-C particles.

In specific embodiments, the saponin matrix composition includes Matrix-A particles and Matrix-C particles, wherein the Matrix-A particles and the Matrix-C particles are present at a fixed weight ratio in the range of about 57:43 to about 53:47, or in the range of about 59:41 to about 51:49, of the Matrix-A particles to the Matrix-C particles.

In specific embodiments, the saponin matrix composition includes Matrix-A particles and Matrix-C particles, wherein the Matrix-A particles and the Matrix-C particles are present at a fixed weight ratio selected from about 60:40, 59:41, 58:42, 57:43, 56:44, 55:45, 54:46, 53:47, 52:48, 51:49, and 50:50 of the Matrix-A particles to the Matrix-C particles.

### Increased Lysosomal Membrane Permeabilization (LMP)

In specific embodiments, administration of the saponin matrix composition together with an antigen result in increased induction of lysosomal membrane permeabilization in antigen-presenting cells compared to administration of a composition having an 85:15 Matrix-A:Matrix-C ratio, under comparable conditions.

In specific embodiments, administration of the saponin matrix composition together with an antigen results in a greater reduction in LysoTracker fluorescence intensity in antigen-presenting cells, and/or a higher proportion of antigen-presenting cells with reduced LysoTracker geometric mean fluorescence intensity in a flow cytometry assay, relative to administration of a composition including Matrix-A particles and Matrix-C particles at an 85:15 weight ratio of Matrix-A particles to Matrix-C particles. In further specific embodiments, the reduction in LysoTracker fluorescence intensity is at least about 20%, specifically at least about 50%, compared to the 85:15 composition, when measured under comparable conditions.

In specific embodiments, administration of the saponin matrix composition together with an antigen results in an increased proportion of antigen-presenting cells that lose LysoTracker staining over time, while maintaining viability dye exclusion, indicative of lysosomal membrane permeabilization without overt cell death, relative to administration of a composition including Matrix-A particles and Matrix-C particles at an 85:15 weight ratio of Matrix-A particles to Matrix-C particles.

In specific embodiments, administration of the saponin matrix composition together with an antigen results in increased release of fluorescently labeled dextran from lysosomes into the cytosol of antigen-presenting cells, as evidenced by a higher proportion of cells exhibiting cytosolic fluorescence of a lysosomally loaded dextran tracer, an increased rate of transition from punctate lysosomal dextran staining to diffuse cytosolic dextran staining, and/or a greater proportion of cells classified as having disrupted lysosomes in a live-cell imaging assay, relative to administration of a composition including Matrix-A particles and Matrix-C particles at an 85:15 weight ratio of Matrix-A particles to Matrix-C particles. In further specific embodiments, there is at least about a 25% increase in the proportion of antigen-presenting cells exhibiting cytosolic fluorescence of a lysosomally loaded dextran tracer and/or at least about a 20% greater reduction over time in the percentage of antigen-presenting cells with intact lysosomes, relative to the 85:15 composition.

In specific embodiments, administration of the saponin matrix composition together with an antigen results in a detectable increase in lysosomal membrane permeabilization in at least one subset of antigen-presenting cells selected from dendritic cells and macrophages, and the frequency of antigen-presenting cells with lysosomal membrane permeabilization is partially inhibited by a V-ATPase inhibitor, relative to administration of a composition including Matrix-A particles and Matrix-C particles at an 85:15 weight ratio of Matrix-A particles to Matrix-C particles.

### Antigen Cross-Presentation Enhancement

In specific embodiments, administration of the saponin matrix composition together with an antigen elicits increased cross-presentation of the antigen (e.g., increased SIINFEKL-H-2K\hat{ }b complexes, B3Z readout) in antigen-presenting cells compared to the 85:15 Matrix-M composition.

In specific embodiments, administration of the saponin matrix composition together with an antigen elicits increased cross-presentation of the antigen in antigen-presenting cells compared to administration of a composition including Matrix-A particles and Matrix-C particles at an 85:15 weight ratio of Matrix-A particles to Matrix-C particles.

In specific embodiments, administration of the saponin matrix composition together with ovalbumin elicits an increased proportion of antigen-presenting cells that are positive for SIINFEKL-H-2K\hat{ }b complexes compared to administration of a composition including Matrix-A particles and Matrix-C particles at an 85:15 weight ratio of Matrix-A particles to Matrix-C particles.

In specific embodiments, administration of the saponin matrix composition together with ovalbumin elicits at least about a 25% increase in the proportion of antigen-presenting cells that are positive for SIINFEKL-H-2K\hat{ }b complexes relative to administration of a composition including Matrix-A particles and Matrix-C particles at an 85:15 weight ratio of Matrix-A particles to Matrix-C particles, when measured under comparable conditions.

In specific embodiments, administration of the saponin matrix composition together with ovalbumin elicits at least about a 50% increase in the proportion of antigen-presenting cells that are positive for SIINFEKL-H-2K\hat{ }b complexes relative to administration of a composition including Matrix-A particles and Matrix-C particles at an 85:15 weight ratio of Matrix-A particles to Matrix-C particles, when measured under comparable conditions.

In specific embodiments, administration of the saponin matrix composition together with an antigen elicits increased cross-presentation of the antigen as measured by an increased frequency of antigen-presenting cells displaying antigen-derived peptide-MHC class I complexes on their surface relative to administration of a composition including Matrix-A particles and Matrix-C particles at an 85:15 weight ratio of Matrix-A particles to Matrix-C particles.

In specific embodiments, administration of the saponin matrix composition together with ovalbumin elicits increased activation of B3Z reporter T cells in a co-culture assay, indicative of enhanced SIINFEKL-H-2K\hat{ }b presentation, relative to administration of a composition including Matrix-A particles and Matrix-C particles at an 85:15 weight ratio of Matrix-A particles to Matrix-C particles.

In specific embodiments, administration of the saponin matrix composition together with ovalbumin elicits at least about a 30% increase in B3Z reporter T-cell activity, as measured by β-galactosidase readout, relative to administration of a composition including Matrix-A particles and Matrix-C particles at an 85:15 weight ratio of Matrix-A particles to Matrix-C particles.

In specific embodiments, administration of the saponin matrix composition together with an antigen elicits increased cross-presentation of the antigen in immature dendritic cells compared to administration of a composition including Matrix-A particles and Matrix-C particles at an 85:15 weight ratio of Matrix-A particles to Matrix-C particles.

In specific embodiments, administration of the saponin matrix composition together with an antigen elicits increased cross-presentation of the antigen in mature dendritic cells, as evidenced by a higher proportion of CD11c\hat{ }+ MHCII\hat{ }hi cells that are positive for antigen-derived peptide-MHC class I complexes, relative to administration of a composition including Matrix-A particles and Matrix-C particles at an 85:15 weight ratio of Matrix-A particles to Matrix-C particles.

In specific embodiments, administration of the saponin matrix composition together with an antigen elicits increased cross-presentation of the antigen in at least one dendritic cell subset selected from cDC1 and cDC2, relative to administration of a composition including Matrix-A particles and Matrix-C particles at an 85:15 weight ratio of Matrix-A particles to Matrix-C particles.

In specific embodiments, administration of the saponin matrix composition together with an antigen elicits increased antigen cross-presentation that is sensitive to inhibition of lysosomal acidification, indicating involvement of lysosomal processing, and the magnitude of the cross-presentation response exceeds that observed after administration of a composition including Matrix-A particles and Matrix-C particles at an 85:15 weight ratio of Matrix-A particles to Matrix-C particles.

In specific embodiments, administration of the saponin matrix composition together with an antigen elicits increased antigen cross-presentation that is sensitive to inhibition of proteasomal activity, and the level of cross-presentation under uninhibited conditions is higher than that induced by administration of a composition including Matrix-A particles and Matrix-C particles at an 85:15 weight ratio of Matrix-A particles to Matrix-C particles.

In specific embodiments, administration of the saponin matrix composition together with an antigen elicits increased cross-presentation of the antigen as evidenced by a higher percentage of antigen-presenting cells that both express activation markers and display antigen-derived peptide-MHC class I complexes, relative to administration of a composition including Matrix-A particles and Matrix-C particles at an 85:15 weight ratio of Matrix-A particles to Matrix-C particles.

In specific embodiments, administration of the saponin matrix composition together with an antigen elicits increased cross-presentation of the antigen at lower antigen doses than those required when using a composition including Matrix-A particles and Matrix-C particles at an 85:15 weight ratio of Matrix-A particles to Matrix-C particles to achieve a comparable level of antigen-derived peptide-MHC class I complex display.

In specific embodiments, administration of the saponin matrix composition together with an antigen elicits increased cross-presentation of the antigen over a broader range of antigen doses than that supported by administration of a composition including Matrix-A particles and Matrix-C particles at an 85:15 weight ratio of Matrix-A particles to Matrix-C particles.

### Antigen-Specific CD8 T-Cell Response Magnitude

In specific embodiments, administration of the saponin matrix composition together with an antigen elicits increased cross-presentation of the antigen in antigen-presenting cells compared to administration of a composition having an 85:15 Matrix-A:Matrix-C ratio, under comparable conditions.

In specific embodiments, administration of the saponin matrix composition together with ovalbumin elicits an increased proportion of antigen-presenting cells that are positive for SIINFEKL-H-2K^{b} complexes compared to administration of a composition including Matrix-A particles and Matrix-C particles at an 85:15 weight ratio of Matrix-A particles to Matrix-C particles. In further specific embodiments, at least about a 25% increase, and specifically at least about a 50% increase, in the proportion of antigen-presenting cells that are positive for SIINFEKL-H-2K^{b} complexes is observed relative to the 85:15 composition, when measured under comparable conditions.

In specific embodiments, administration of the saponin matrix composition together with an antigen elicits increased cross-presentation of the antigen as measured by an increased frequency of antigen-presenting cells displaying antigen-derived peptide-MHC class I complexes on their surface and/or increased activation of B3Z reporter T cells in a co-culture assay, indicative of enhanced SIINFEKL-H-2K^{b} presentation, relative to administration of a composition including Matrix-A particles and Matrix-C particles at an 85:15 weight ratio of Matrix-A particles to Matrix-C particles. In further specific embodiments, B3Z reporter T-cell activity, as measured by β-galactosidase readout, is increased by at least about 30% relative to the 85:15 composition.

In specific embodiments, administration of the saponin matrix composition together with an antigen elicits increased cross-presentation of the antigen in immature dendritic cells and/or in mature dendritic cells, as evidenced by a higher proportion of CD11c⁺ MHCII^{hi} cells that are positive for antigen-derived peptide-MHC class I complexes, and in at least one dendritic cell subset selected from cDC1 and cDC2, relative to administration of a composition including Matrix-A particles and Matrix-C particles at an 85:15 weight ratio of Matrix-A particles to Matrix-C particles.

In specific embodiments, administration of the saponin matrix composition together with an antigen elicits increased antigen cross-presentation that is sensitive to inhibition of lysosomal acidification and/or proteasomal activity, and the magnitude of the cross-presentation response under uninhibited conditions exceeds that observed after administration of a composition including Matrix-A particles and Matrix-C particles at an 85:15 weight ratio of Matrix-A particles to Matrix-C particles.

In specific embodiments, administration of the saponin matrix composition together with an antigen elicits increased cross-presentation of the antigen at lower antigen doses and/or over a broader range of antigen doses than that supported by administration of a composition including Matrix-A particles and Matrix-C particles at an 85:15 weight ratio of Matrix-A particles to Matrix-C particles, to achieve a comparable level of antigen-derived peptide-MHC class I complex display.

Vaccine Composition with Defined Antigen Classes

In specific embodiments, the vaccine composition includes: (i) a saponin matrix composition that includes Matrix-A particles and Matrix-C particles at a fixed weight ratio in the range of about 70:30 to 10:90 of Matrix-A particles to Matrix-C particles (optionally in the range of about 57:43 to about 53:47); and (ii) an antigen selected from viral antigens (e.g., SARS-CoV-2 S, RSV F0, PIV3, hMPV F) and tumor antigens, including tumor-associated antigens (TAAs) and tumor neoantigens as exemplified herein.

In specific embodiments, the vaccine composition includes a saponin matrix composition that includes Matrix-A particles and Matrix-C particles at a fixed weight ratio in the range of about 70:30 to 10:90 of Matrix-A particles to Matrix-C particles and further includes a viral antigen, such as a SARS-CoV-2 spike protein antigen, a parainfluenza virus type 3 antigen, or a human metapneumovirus F protein antigen.

In specific embodiments, the vaccine composition includes a saponin matrix composition that includes Matrix-A particles and Matrix-C particles at a fixed weight ratio in the range of about 70:30 to 10:90 of Matrix-A particles to Matrix-C particles and further includes a tumor-associated antigen, such as a TAA selected from HPV-16 E6, HPV-16 E7, HPV-18 E6, HPV-18 E7, melanoma-associated antigen family members, and prostate-specific antigen, and/or a tumor neoantigen selected from KRAS G12D, KRAS G12V, KRAS G12C, KRAS G12A, KRAS G13D, BRAF V600E, PIK3CA H1047R, and TP53R175H.

In specific embodiments, the vaccine composition includes a saponin matrix composition that includes Matrix-A particles and Matrix-C particles at a fixed weight ratio in the range of about 70:30 to 10:90 of Matrix-A particles to Matrix-C particles and further includes at least one viral antigen selected from SARS-CoV-2 spike protein I, respiratory syncytial virus F0 protein, parainfluenza virus type 3 antigen, and human metapneumovirus F protein, together with at least one tumor-associated antigen or tumor neoantigen.

### Antigen-to-Matrix Weight Ratios

In specific embodiments, the antigen is present at fixed weight ratios relative to the total weight of Matrix-A plus Matrix-C and/or to Matrix-C alone (e.g., from about 0.1 μg:200 μg to about 400 μg:20 μg, and from about 5 μg:100 μg to about 400 μg:50 μg).

In specific embodiments, the vaccine composition includes an antigen and a saponin matrix composition that includes Matrix-A particles and Matrix-C particles, and the antigen is present at a fixed weight ratio to the total weight of Matrix-A particles plus Matrix-C particles in the range of about 0.1 μg antigen to about 200 μg of Matrix-A particles plus Matrix-C particles up to about 400 μg antigen to about 20 μg of Matrix-A particles plus Matrix-C particles. In further specific embodiments, the antigen is present at a fixed weight ratio to the total weight of Matrix-A particles plus Matrix-C particles of about 0.1 μg:200 μg, about 1 μg:100 μg, about 5 μg:100 μg, about 10 μg:50 μg, about 50 μg:50 μg, or about 100 μg:50 μg (antigen:total Matrix-A plus Matrix-C).

In specific embodiments, the vaccine composition includes an antigen and a saponin matrix composition that includes Matrix-A particles and Matrix-C particles, and the antigen is present at a fixed weight ratio to the total weight of Matrix-A particles plus Matrix-C particles in the range of about 5 μg antigen to about 100 μg of Matrix-A particles plus Matrix-C particles up to about 400 μg antigen to about 50 μg of Matrix-A particles plus Matrix-C particles.

In specific embodiments, the vaccine composition includes an antigen and a saponin matrix composition that includes Matrix-A particles and Matrix-C particles, and the antigen is present at a fixed weight ratio to Matrix-C alone in the range of about 0.1 μg antigen to about 20 μg Matrix-C up to about 400 μg antigen to about 5 μg Matrix-C. In further specific embodiments, the antigen is present at a fixed weight ratio to Matrix-C alone of about 0.1 μg:20 μg, about 1 μg:10 μg, about 5 μg:10 μg, about 50 μg:10 μg, about 100 μg:10 μg, or about 400 μg:50 μg (antigen:Matrix-C).

### Dosage of Matrix Components Per Administration

In specific embodiments, the saponin matrix composition is administered at a dosage of about 20 μg to about 200 μg of Matrix-C, or about 20 μg to about 200 μg of the total of Matrix-A plus Matrix-C, per administration, optionally with specific subranges of about 50 μg to about 100 μg or about 25 μg to about 150 μg.

In specific embodiments, the saponin matrix composition is administered at a dosage of about 20 μg, about 50 μg, about 75 μg, about 100 μg, about 150 μg, or about 200 μg of Matrix-C per administration.

In specific embodiments, the saponin matrix composition is administered at a dosage of about 20 μg, about 50 μg, about 75 μg, about 100 μg, about 150 μg, or about 200 μg of the total of Matrix-A plus Matrix-C per administration.

In specific embodiments, the saponin matrix composition is administered at a dosage of about 20 μg to about 200 μg of the total of Matrix-A plus Matrix-C per administration, wherein the Matrix-A particles and Matrix-C particles are present at a fixed weight ratio in the range of about 70:30 to 10:90 of Matrix-A particles to Matrix-C particles.

### Formulation State and Buffer Conditions

In specific embodiments, the saponin matrix composition and/or the vaccine composition is formulated as an aqueous composition in phosphate buffer including sodium chloride and potassium chloride at a pH of about 7.2, and/or as a lyophilized or spray-dried solid composition that is reconstitutable for parenteral, oral, or inhalation administration.

In specific embodiments, the saponin matrix composition and/or the vaccine composition is formulated as an aqueous isotonic phosphate-buffered saline composition including about 137 mM sodium chloride, about 2.7 mM potassium chloride, and about 9.8 mM phosphate at a pH of about 7.2, or at a pH in the range of about 7.0 to about 7.4.

In specific embodiments, the saponin matrix composition and/or the vaccine composition is provided as a lyophilized solid that is reconstitutable in an aqueous diluent (optionally the same phosphate-buffered saline) for parenteral administration, and/or as a spray-dried powder that is reconstitutable in an aqueous diluent for parenteral administration.

In specific embodiments, the saponin matrix composition and/or the vaccine composition is provided as a spray-dried powder suitable for oral administration after reconstitution or dispersion in an ingestible liquid, and/or as a spray-dried powder suitable for inhalation administration using a dry powder inhaler or after reconstitution in an inhalable liquid formulation.

In specific embodiments, the vaccine composition is formulated as an aqueous composition in phosphate buffer including sodium chloride and potassium chloride at a pH of about 7.2 and is alternatively provided as a lyophilized solid that is reconstitutable in the same buffer for administration.

### Route and Mode of Administration

In specific embodiments, the saponin matrix composition (alone or together with an antigen) is administered parenterally, especially by intramuscular, subcutaneous, or intradermal injection, including administration as a single co-formulated injection or as separate compositions given at the same or anatomically proximate site within a defined time window.

In specific embodiments, the saponin matrix composition and/or a vaccine composition that includes the saponin matrix composition and an antigen is administered to a subject by intramuscular, subcutaneous, or intradermal injection in a single injection volume.

In specific embodiments, the saponin matrix composition and the antigen are administered to a subject in separate compositions by intramuscular, subcutaneous, or intradermal injection at the same anatomical site, or at anatomically proximate sites within a time window of about 0 to about 60 minutes, optionally within about 0 to about 30 minutes and optionally on the same limb.

In specific embodiments, the saponin matrix composition is administered to a subject by intranasal administration and/or by pulmonary inhalation.

Cancer Treatment Use with Defined Tumor Antigens

In specific embodiments, methods of treating cancer in a subject include administering to the subject a tumor antigen (tumor-associated antigen and/or tumor neoantigen) together with a saponin matrix composition that includes Matrix-A particles and Matrix-C particles at a fixed weight ratio in the range of about 70:30 to 10:90 of Matrix-A particles to Matrix-C particles.

In specific embodiments, the tumor-associated antigen is selected from HPV-16 E6, HPV-16 E7, HPV-18 E6, HPV-18 E7, melanoma-associated antigen family members, and prostate-specific antigen, and is administered together with a saponin matrix composition that includes Matrix-A particles and Matrix-C particles at a fixed weight ratio in the range of about 70:30 to 10:90 of Matrix-A particles to Matrix-C particles.

In specific embodiments, the tumor neoantigen is selected from KRAS G12D, KRAS G12V, KRAS G12C, KRAS G12A, KRAS G13D, BRAF V600E, PIK3CA H1047R, TP53 R175H, and TP53 R273H, and is administered together with a saponin matrix composition that includes Matrix-A particles and Matrix-C particles at a fixed weight ratio in the range of about 70:30 to 10:90 of Matrix-A particles to Matrix-C particles.

In specific embodiments, the method of treating cancer in a subject includes administering to the subject a tumor antigen composition that includes multiple tumor neoantigen peptides selected from KRAS, BRAF, PIK3CA, and TP53 mutations, and/or at least one tumor-associated antigen selected from melanoma-associated antigen family members and at least one tumor neoantigen selected from KRAS G12D and BRAF V600E, together with a saponin matrix composition that includes Matrix-A particles and Matrix-C particles at a fixed weight ratio in the range of about 70:30 to 10:90 of Matrix-A particles to Matrix-C particles.

In specific embodiments, the method of treating cancer in a subject includes administering to the subject a personalized tumor antigen composition that includes one or more tumor neoantigens identified in that subject's tumor and a saponin matrix composition that includes Matrix-A particles and Matrix-C particles at a fixed weight ratio in the range of about 70:30 to 10:90 of Matrix-A particles to Matrix-C particles.

In specific embodiments, the method of treating cancer in a subject includes administering to the subject a tumor antigen composition that includes at least one tumor-associated antigen and at least one tumor neoantigen together with a saponin matrix composition that includes Matrix-A particles and Matrix-C particles at a fixed weight ratio of Matrix-A particles to Matrix-C particles, thereby inducing antigen-specific CD8 T-cell responses against the tumor.

### Inducing Adaptive Immunity to Defined Viral Pathogens

In specific embodiments, methods for inducing an adaptive immune response or protective immunity against viral pathogens, including SARS-CoV-2, parainfluenza virus type 3 (PIV3), respiratory syncytial virus (RSV), and human metapneumovirus (hMPV), are achieved by administering to a subject a viral antigen together with a saponin matrix composition that includes Matrix-A particles and Matrix-C particles at a fixed weight ratio in the range of about 70:30 to 10:90 of Matrix-A particles to Matrix-C particles.

In specific embodiments, the viral antigen is a SARS-CoV-2 spike protein antigen, a respiratory syncytial virus F0 protein antigen, a parainfluenza virus type 3 antigen, or a human metapneumovirus F protein antigen, and is administered together with a saponin matrix composition that includes Matrix-A particles and Matrix-C particles at a fixed weight ratio in the range of about 70:30 to 10:90 of Matrix-A particles to Matrix-C particles.

In specific embodiments, methods for inducing an adaptive immune response or protective immunity against one or more respiratory viral pathogens selected from SARS-CoV-2, respiratory syncytial virus, parainfluenza virus type 3, and human metapneumovirus in a subject include administering to the subject at least one corresponding viral antigen together with a saponin matrix composition that includes Matrix-A particles and Matrix-C particles at a fixed weight ratio in the range of about 70:30 to 10:90 of Matrix-A particles to Matrix-C particles.

In specific embodiments, methods for inducing an adaptive immune response against both a respiratory viral pathogen and a non-respiratory viral pathogen in a subject include administering to the subject at least one respiratory viral antigen selected from SARS-CoV-2 spike protein, respiratory syncytial virus F0 protein, parainfluenza virus type 3 antigen, and human metapneumovirus F protein, together with a saponin matrix composition that includes Matrix-A particles and Matrix-C particles at a fixed weight ratio in the range of about 70:30 to 10:90 of Matrix-A particles to Matrix-C particles.

### Activation of Antigen-Presenting Cell Subsets

In specific embodiments, methods for activating antigen-presenting cells in a subject include administering to the subject an antigen together with a saponin matrix composition that includes Matrix-A particles and Matrix-C particles at a fixed weight ratio in the range of about 70:30 to 10:90 of Matrix-A particles to Matrix-C particles, and the administration results in increased activation of antigen-presenting cells compared to administration of the antigen with a composition including Matrix-A particles and Matrix-C particles at an 85:15 weight ratio of Matrix-A particles to Matrix-C particles.

In specific embodiments, the activated antigen-presenting cells include dendritic cells, macrophages, B cells, and/or specific dendritic cell subsets such as conventional dendritic cell type 1 (cDC1), conventional dendritic cell type 2 (cDC2), inflammatory cDC2, and monocyte-derived dendritic cells, and activation is increased relative to administration of the antigen with a composition including Matrix-A particles and Matrix-C particles at an 85:15 weight ratio of Matrix-A particles to Matrix-C particles.

In specific embodiments, administration of an antigen together with a saponin matrix composition that includes Matrix-A particles and Matrix-C particles at a fixed weight ratio in the range of about 70:30 to 10:90 of Matrix-A particles to Matrix-C particles results in increased expression of MHCII and/or CD86 on antigen-presenting cells, an increased proportion of dendritic cells that are MHCII^{hi} and CD86^{hi}, and increased activation of CD11c⁺ MHCII⁺ antigen-presenting cells in draining lymph nodes, compared to administration of the antigen with a composition including Matrix-A particles and Matrix-C particles at an 85:15 weight ratio of Matrix-A particles to Matrix-C particles.

In specific embodiments, administration of an antigen together with a saponin matrix composition results in increased activation of antigen-presenting cells that are CD88⁺ CD64⁺ monocyte-derived dendritic cells and/or CD26⁺ CD64⁻ conventional dendritic cells, including XCR1⁺ cDC1 and CD172a⁺ cDC2 subsets, and in increased activation of at least one antigen-presenting cell subset selected from dendritic cells, macrophages, B cells, cDC1, cDC2, inflammatory cDC2, and monocyte-derived dendritic cells, as evidenced by upregulation of MHCII and/or CD86, compared to administration of the antigen with a composition including Matrix-A particles and Matrix-C particles at an 85:15 weight ratio of Matrix-A particles to Matrix-C particles.

NLRP3-Independent Adjuvanticity with Defined Innate Readouts

In specific embodiments, administration of an antigen together with a saponin matrix composition induces IL-1β secretion, IL-18 secretion, lysosomal membrane permeabilization, and other inflammasome-related events in antigen-presenting cells, including loss of LysoTracker staining and release of lysosomal tracers into the cytosol.

In specific embodiments, administration of an antigen together with a saponin matrix composition induces IL-1β secretion from antigen-presenting cells in vitro, which is sensitive to inhibition by an NLRP3 inflammasome inhibitor, and/or induces IL-18 secretion from antigen-presenting cells that is not substantially reduced by inhibition of NLRP3 inflammasome assembly in vitro. In further specific embodiments, lysosomal membrane permeabilization and IL-1β secretion in antigen-presenting cells are induced to a greater extent than upon administration of the antigen with a composition including Matrix-A particles and Matrix-C particles at an 85:15 weight ratio of Matrix-A particles to Matrix-C particles.

In specific embodiments, methods for inducing an adjuvant effect include administering to a subject an antigen together with a saponin matrix composition, and the administration induces IL-1β and/or IL-18 secretion and lysosomal membrane permeabilization in antigen-presenting cells.

In specific embodiments, methods for inducing an adjuvant effect that is NLRP3-independent include administering to wild-type and Nlrp3-deficient subjects an antigen together with a saponin matrix composition, and comparable antigen-specific antibody and T-cell responses are observed in the wild-type and Nlrp3-deficient subjects. In further specific embodiments, antigen-specific CD8 T-cell responses producing IFN-γ, antigen-specific antibody titers, and frequencies of antigen-specific CD4 and CD8 T cells producing IFN-γ and/or IL-2 are not substantially reduced in Nlrp3-deficient subjects relative to wild-type subjects, such that the adjuvant effect, as measured by antigen-specific antibody titers and T-cell responses, is maintained in the absence of NLRP3 inflammasome activity in vivo.

### Co-Localization and Trafficking Features (Lysosomal Targeting)

In specific embodiments, Matrix-A particles and Matrix-C particles in a saponin matrix composition, upon uptake by antigen-presenting cells, accumulate in lysosomes as evidenced by detectable co-localization of Matrix-associated fluorescence with LysoTracker fluorescence and, when a fluorescently labeled antigen is present, by co-localization of Matrix-associated fluorescence with antigen-associated fluorescence in LysoTracker-positive compartments.

In specific embodiments, Matrix-A particles and Matrix-C particles in a saponin matrix composition, upon uptake by antigen-presenting cells, show co-localization with LysoTracker fluorescence having a Pearson's correlation coefficient above about 0.3 over at least about 60-90 minutes after addition to the cells, and in further specific embodiments having a Pearson's correlation coefficient above about 0.5 at one or more time points within about 120 minutes after addition. In specific embodiments, Matrix-A particles and Matrix-C particles labeled with a fluorescent probe show co-localization kinetics in which Pearson's correlation coefficients for Matrix-lysosome co-localization peak between about 30 minutes and about 90 minutes after addition.

In specific embodiments, Matrix-A particles and Matrix-C particles in a saponin matrix composition, upon uptake by antigen-presenting cells, exhibit higher Pearson's correlation coefficients for co-localization with LysoTracker fluorescence than Matrix-A particles or Matrix-C particles tested alone, and/or higher co-localization of Matrix-associated fluorescence with LysoTracker-loaded lysosomes and with fluorescent antigen than a composition including Matrix-A particles and Matrix-C particles at an 85:15 weight ratio of Matrix-A particles to Matrix-C particles.

In specific embodiments, a saponin matrix composition that includes Matrix-A particles and Matrix-C particles at a fixed weight ratio in the range of about 70:30 to 10:90 of Matrix-A particles to Matrix-C particles upon uptake by antigen-presenting cells exhibits co-localization with LysoTracker fluorescence that correlates with enhanced antigen cross-presentation and increased formation of antigen-derived peptide-MHC class I complexes, and with enhanced antigen-specific CD8 T-cell priming in vivo, relative to a composition including Matrix-A particles and Matrix-C particles at an 85:15 weight ratio of Matrix-A particles to Matrix-C particles.

In specific embodiments, a saponin matrix composition that includes Matrix-A particles and Matrix-C particles, upon uptake by antigen-presenting cells, exhibits a pattern of lysosomal targeting such that the majority of Matrix-associated fluorescent signal overlaps with LysoTracker signal at multiple time points, co-localization of Matrix-associated fluorescence with LysoTracker fluorescence and with fluorescent antigen is detectable for at least about 120 minutes, and this lysosomal targeting is associated with increased antigen-specific CD8 T-cell responses in vivo relative to a composition including Matrix-A particles and Matrix-C particles at an 85:15 weight ratio of Matrix-A particles to Matrix-C particles.

### Enumerated Embodiments

Specific enumerated embodiments <1> to <68> provided below are for illustration purposes only, and do not otherwise limit the scope of the disclosed subject matter, as defined by the claims. These enumerated embodiments encompass all combinations, sub-combinations, and multiply referenced (e.g., multiply dependent) combinations described therein.
<Embodiment 1> A saponin matrix composition comprising Matrix-A particles and Matrix-C particles, wherein the Matrix-A particles and the Matrix-C particles are present at a fixed weight ratio in the range of 70:30 to 10:90 of the Matrix-A particles to the Matrix-C particles.
<Embodiment 2> The saponin matrix composition according to Embodiment <1>, wherein the fixed weight ratio of the Matrix-A particles to the Matrix-C particles is about 60:40, 59:41, 58:42, 57:43, 56:44, 55:45, 54:46, 53:47, 52:48, 51:49, or 50:50.
<Embodiment 3> The saponin matrix composition according to any one of Embodiments <1> to <2>, wherein the composition, when administered together with an antigen to a subject, induces an increased production of IFN-γ by antigen-specific CD8 T cells under comparable dosing and administration conditions compared to a saponin matrix composition having a weight ratio of 85:15 of the Matrix-A particles to the Matrix-C particles.
<Embodiment 4> The saponin matrix composition according to any one of Embodiments <1> to <3>, wherein, when used as an adjuvant for immunization of a mouse with a SARS-CoV-2 rS protein antigen, the composition leads to an elevated frequency of antigen-specific CD8 T-cell IFN-γ responses as detected ex vivo by restimulation of splenocytes from the immunized mouse with the antigen, compared to a saponin matrix composition having a weight ratio of 85:15 of the Matrix-A particles to the Matrix-C particles under comparable immunization conditions.
<Embodiment 5> The saponin matrix composition according to any one of Embodiments <1> to <4>, wherein Matrix-A particles and Matrix-C particles are prepared from saponin Fraction-A and saponin Fraction-C of Quillaja saponaria Molina obtained by reversed-phase high-performance liquid chromatography of a purified Quillaja saponin extract, followed by formation of lipid-containing particles with cholesterol and phosphatidylcholine.
<Embodiment 6> The saponin matrix composition according to any one of Embodiments <1> to <5>, wherein the Matrix-A particles and the Matrix-C particles are provided in an aqueous composition in phosphate buffer including sodium chloride and potassium chloride at a pH of about 7.2.
<Embodiment 7> The saponin matrix composition according to any one of Embodiments <1> to <6>, wherein the composition is provided as an aqueous composition that has been subjected to spray drying or lyophilization to yield a solid composition, the solid composition being formulated into a solid dosage form or reconstituted in a pharmaceutically acceptable liquid vehicle to provide a liquid dosage form suitable for administration to a subject.
<Embodiment 8> The saponin matrix composition according to any one of Embodiments <1> to <7>, wherein administration of the composition together with an antigen to a subject results in increased antigen cross-presentation compared to administration of a saponin matrix composition having a weight ratio of 85:15 of the Matrix-A particles to the Matrix-C particles.
<Embodiment 9> The saponin matrix composition according to any one of Embodiments <1> to <8>, wherein administration of the composition together with an antigen to a subject results in increased antigen cross-presentation compared to administration of a saponin matrix composition having a weight ratio of 85:15 of the Matrix-A particles to the Matrix-C particles, and wherein the increased antigen cross-presentation in the subject involves increased induction of lysosomal membrane permeabilization in antigen-presenting cells compared to administering a vaccine composition comprising the antigen and a saponin matrix composition having a weight ratio of 85:15 of the Matrix-A particles to the Matrix-C particles under comparable dosing and administration conditions.
<Embodiment 10> The saponin matrix composition according to any one of Embodiments <1> to <9>, wherein administration of the composition together with an antigen to a subject results in increased activation of antigen-presenting cells compared to administration of a saponin matrix composition having a weight ratio of 85:15 of the Matrix-A particles to the Matrix-C particles.
<Embodiment 11> The saponin matrix composition according to any one of Embodiments <1> to <10>, wherein the composition, when administered together with an antigen to a subject, induces an increase in antigen-specific CD8 T-cell responses by at least 30%, by at least 50%, by at least 75%, or by at least 100%, relative to a saponin matrix composition having a weight ratio of 85:15 of the Matrix-A particles to the Matrix-C particles when administered under comparable conditions.
<Embodiment 12> The saponin matrix composition according to any one of Embodiments <1> to <11>, wherein the composition is for use as an adjuvant in a vaccine composition comprising a protein antigen selected from a SARS-CoV-2 S protein antigen, a respiratory syncytial virus F protein antigen, a parainfluenza virus type 3 antigen, or a human metapneumovirus F antigen.
<Embodiment 13> The saponin matrix composition according to any one of Embodiments <1> to <12>, wherein the composition is an aqueous composition, is in a lyophilized form, or is in a spray dried form.
<Embodiment 14> A vaccine composition comprising an antigen and a saponin matrix composition of any one of Embodiments <1> to <13>.
<Embodiment 15> The vaccine composition according to Embodiment <14>, wherein the antigen is a viral antigen selected from a SARS-CoV-2 S protein antigen, a parainfluenza virus type 3 antigen, a respiratory syncytial virus F0 antigen, and a human metapneumovirus F antigen.
<Embodiment 16> The vaccine composition according to Embodiment <14>, wherein the antigen is a tumor neoantigen selected from KRAS G12D, KRAS G12V, KRAS G12C, KRAS G12A, KRAS G13D, BRAF V600E, PIK3CA H1047R, TP53 R175H, TP53R273H, SYT-SSX1, SYT-SSX2, PAX3-FOXO1, EWS-FLI1, BCR-ABL b3a2, BCR-ABL b2a2, CCDC6-RET, FGFR3-TACC3, TPM3-ALK, TPM4-ALK, NTRK1 fusion TPM3-NTRK1, ETV6-NTRK3, DEK-AFF2, TGFBR2 frameshift-1A, ACVR2A frameshift-1A, CASP5 frameshift, TAF1B frameshift, ASTE1/HT001 frameshift, or wherein the antigen is a tumor-associated antigen selected from HPV-16 E6, HPV-16 E7, HPV-18 E6, HPV-18 E7, melanoma-associated antigen MAGE family antigens, and prostate-specific antigen PSA.
<Embodiment 17> The vaccine composition according to any one of Embodiments <14> to <16>, wherein the antigen is present at a fixed weight ratio from 0.1 μg to 200 μg of the antigen per 400 μg to 20 μg of the total weight of the Matrix-A particles and the Matrix-C particles.
<Embodiment 18> The vaccine composition according to any one of Embodiments <14> to <16>, wherein the antigen is present at a fixed weight ratio from 5 μg to 100 μg of the antigen per 400 μg to 50 μg of the total weight of the Matrix-A particles and the Matrix-C particles.
<Embodiment 19> The vaccine composition according to any one of Embodiments <14> to <18>, wherein, when administered to a subject, the vaccine composition induces an increased production of IFN-γ by antigen-specific CD8 T cells compared to a vaccine composition comprising an antigen and a saponin matrix composition having a weight ratio of 85:15 of the Matrix-A particles to the Matrix-C particles when administered under comparable dosing and schedule.
<Embodiment 20> The vaccine composition according to any one of Embodiments <14> to <19>, wherein, when used to immunize a mouse with a SARS-CoV-2 rS protein antigen, the vaccine composition leads to an elevated frequency of antigen-specific CD8 T-cell IFN-γ responses as detected ex vivo by restimulation of splenocytes from the immunized mouse with the antigen, compared to a vaccine composition comprising the antigen and a saponin matrix composition having a weight ratio of 85:15 of the Matrix-A particles to the Matrix-C particles under comparable immunization conditions.
<Embodiment 21> The vaccine composition according to any one of Embodiments <14> to <20>, wherein the vaccine composition is formulated as an aqueous composition in phosphate buffer including sodium chloride and potassium chloride at a pH of about 7.2.
<Embodiment 22> The vaccine composition according to any one of Embodiments <14> to <21>, wherein the vaccine composition is provided as an aqueous composition that has been subjected to spray drying or lyophilization to yield a solid composition, the solid composition being formulated into a solid dosage form or reconstituted in a pharmaceutically acceptable liquid vehicle to provide a liquid dosage form suitable for parenteral, oral, or inhalation administration.
<Embodiment 23> A method of administering an antigen to a subject in need thereof, the method comprising administering to the subject an effective amount of an antigen and an effective amount of a saponin matrix composition of any one of Embodiments <1> to <13>.
<Embodiment 24> The method according to Embodiment <23>, wherein the antigen is a viral antigen selected from a SARS-CoV-2 S protein antigen, a parainfluenza virus type 3 antigen, a respiratory syncytial virus F0 antigen, and a human metapneumovirus F antigen.
<Embodiment 25> The method according to any one of Embodiments <23> to <24>, wherein the antigen is a tumor neoantigen selected from KRAS G12D, KRAS G12V, KRAS G12C, KRAS G12A, KRAS G13D, BRAF V600E, PIK3CA H1047R, TP53 R175H, TP53 R273H, SYT-SSX1, SYT-SSX2, PAX3-FOX01, EWS-FLI1, BCR-ABL b3a2, BCR-ABL b2a2, CCDC6-RET, FGFR3-TACC3, TPM3-ALK, TPM4-ALK, NTRK1 fusion TPM3-NTRK1, ETV6-NTRK3, DEK-AFF2, TGFBR2 frameshift-1A, ACVR2A frameshift-1A, CASP5 frameshift, TAF1B frameshift, ASTE1/HT001 frameshift, or wherein the antigen is a tumor-associated antigen selected from HPV-16 E6, HPV-16 E7, HPV-18 E6, HPV-18 E7, melanoma-associated antigen MAGE family antigens, and prostate-specific antigen PSA.
<Embodiment 26> The method according to any one of Embodiments <23> to <25>, wherein the antigen is present at a fixed weight ratio from 0.1 μg to 200 μg of the antigen per 400 μg to 20 μg of the total weight of the Matrix-A particles and the Matrix-C particles.
<Embodiment 27> The method according to any one of Embodiments <23> to <26>, wherein administering the antigen and the saponin matrix composition to the subject induces an increased production of IFN-γ by antigen-specific CD8 T cells compared to administering a vaccine composition comprising the antigen and a saponin matrix composition having a weight ratio of 85:15 of the Matrix-A particles to the Matrix-C particles under comparable dosing and administration conditions.
<Embodiment 28> The method according to any one of Embodiments <23> to <26>, wherein administering the antigen and the saponin matrix composition to the subject elicits increased antigen cross-presentation compared to administering a vaccine composition comprising the antigen and a saponin matrix composition having a weight ratio of 85:15 of the Matrix-A particles to the Matrix-C particles under comparable dosing and administration conditions.
<Embodiment 29> The method according to any one of Embodiments <23> to <26>, wherein administering the antigen and the saponin matrix composition to the subject results in increased activation of antigen-presenting cells compared to administering a vaccine composition comprising the antigen and a saponin matrix composition having a weight ratio of 85:15 of the Matrix-A particles to the Matrix-C particles under comparable dosing and administration conditions.
<Embodiment 30> The method according to any one of Embodiments <23> to <29>, wherein the antigen and the saponin matrix composition are co-formulated in a single vaccine composition that is administered to the subject as an intramuscular injection.
<Embodiment 31> The method according to any one of Embodiments <23> to <29>, wherein the antigen and the saponin matrix composition are administered to the subject in separate pharmaceutical compositions given at the same or an anatomically proximate site within a time window of up to 24 hours.
<Embodiment 32> The method according to any one of Embodiments <23> to <31>, wherein the saponin matrix composition is administered to the subject at a dosage of 20 μg to 200 μg of Matrix-C particles.
<Embodiment 33> The method according to any one of Embodiments <23> to <31>, wherein the saponin matrix composition is administered to the subject at a dosage of 20 μg to 200 μg of the total weight of the Matrix-A particles and the Matrix-C particles.
<Embodiment 34> A method for inducing production of IFN-γ by antigen-specific CD8 T cells in a subject in need thereof, the method comprising administering to the subject an effective amount of an antigen and an effective amount of a saponin matrix composition of any one of Embodiments <1> to <13>, thereby inducing production of IFN-γ by antigen-specific CD8 T cells in the subject.
<Embodiment 35> The method according to Embodiment <34>, wherein administering the antigen and the saponin matrix composition to the subject induces an increased production of IFN-γ by antigen-specific CD8 T cells compared to administering a vaccine composition comprising the antigen and a saponin matrix composition having a weight ratio of 85:15 of the Matrix-A particles to the Matrix-C particles under comparable dosing and administration conditions.
<Embodiment 36> The method according to Embodiments <34>, wherein the antigen is a viral antigen selected from a SARS-CoV-2 S protein antigen, a parainfluenza virus type 3 antigen, a respiratory syncytial virus F0 antigen, and a human metapneumovirus F antigen.
<Embodiment 37> The method according to Embodiments <34>, wherein the antigen is a tumor neoantigen selected from KRAS G12D, KRAS G12V, KRAS G12C, KRAS G12A, KRAS G13D, BRAF V600E, PIK3CA H1047R, TP53 R175H, TP53 R273H, SYT-SSX1, SYT-SSX2, PAX3-FOX01, EWS-FLI1, BCR-ABL b3a2, BCR-ABL b2a2, CCDC6-RET, FGFR3-TACC3, TPM3-ALK, TPM4-ALK, NTRK1 fusion TPM3-NTRK1, ETV6-NTRK3, DEK-AFF2, TGFBR2 frameshift-1A, ACVR2A frameshift-1A, CASP5 frameshift, TAF1B frameshift, ASTE1/HT001 frameshift, or wherein the antigen is a tumor-associated antigen selected from HPV-16 E6, HPV-16 E7, HPV-18 E6, HPV-18 E7, melanoma-associated antigen MAGE family antigens, and prostate-specific antigen PSA
<Embodiment 38> The method according to any one of Embodiments <34> to <37>, wherein the antigen is present at a fixed weight ratio from 0.1 μg to 200 μg of the antigen per 400 μg to 20 μg of the total weight of the Matrix-A particles and the Matrix-C particles.
<Embodiment 39> The method according to any one of Embodiments <34> to <37>, wherein the antigen is present at a fixed weight ratio from 5 μg to 100 μg of the antigen per 400 μg to 50 μg of the total weight of the Matrix-A particles and the Matrix-C particles.
<Embodiment 40> The method according to any one of Embodiments <34> to <39>, wherein administering the antigen and the saponin matrix composition to the subject results in an increase in the frequency of antigen-specific CD8 T cells producing IFN-γ by at least 30%, specifically at least 50%, more specifically at least 75%, more specifically at least 100%, relative to administering a vaccine composition comprising the antigen and a saponin matrix composition having a weight ratio of 85:15 of the Matrix-A particles to the Matrix-C particles under comparable dosing and administration conditions.
<Embodiment 41> The method according to any one of Embodiments <34> to <39>, wherein administering the antigen and the saponin matrix composition to the subject elicits increased antigen cross-presentation in antigen-presenting cells compared to administering a vaccine composition comprising the antigen and a saponin matrix composition having a weight ratio of 85:15 of the Matrix-A particles to the Matrix-C particles.
<Embodiment 42> The method according to any one of Embodiments <34> to <41>, wherein the antigen and the saponin matrix composition are co-formulated in a single vaccine composition that is administered to the subject as an intramuscular injection.
<Embodiment 43> The method according to any one of Embodiments <34> to <41>, wherein the antigen and the saponin matrix composition are administered to the subject in separate pharmaceutical compositions given at the same anatomical site within a time window of up to 24 hours.
<Embodiment 44> A method of treatment for cancer in a subject in need thereof, the method comprising administering to the subject an effective amount of a tumor antigen and an effective amount of a saponin matrix composition of any one of Embodiments <1> to <13>, thereby treating the cancer in the subject.
<Embodiment 45> The method according to Embodiment <44>, wherein the tumor antigen is a tumor neoantigen selected from KRAS G12D, KRAS G12V, KRAS G12C, KRAS G12A, KRAS G13D, BRAF V600E, PIK3CA H1047R, TP53 R175H, TP53R273H, SYT-SSX1, SYT-SSX2, PAX3-FOX01, EWS-FLI1, BCR-ABL b3a2, BCR-ABL b2a2, CCDC6-RET, FGFR3-TACC3, TPM3-ALK, TPM4-ALK, NTRK1 fusion TPM3-NTRK1, ETV6-NTRK3, DEK-AFF2, TGFBR2 frameshift-1A, ACVR2A frameshift-1A, CASP5 frameshift, TAF1B frameshift, and ASTE1/HT001 frameshift.
<Embodiment 46> The method according to Embodiments <44>, wherein the tumor antigen is a tumor-associated antigen selected from HPV-16 E6, HPV-16 E7, HPV-18 E6, HPV-18 E7, melanoma-associated antigen MAGE family antigens, and prostate-specific antigen PSA.
<Embodiment 47> The method according to any one of Embodiments <44> to <46>, wherein the tumor antigen is present at a fixed weight ratio from 0.1 μg to 200 μg of the tumor antigen per 400 μg to 20 μg of the total weight of the Matrix-A particles and the Matrix-C particles.
<Embodiment 48> The method according to any one of Embodiments <44> to <46>, wherein the tumor antigen is present at a fixed weight ratio from 5 μg to 100 μg of the tumor antigen per 400 μg to 50 μg of the total weight of the Matrix-A particles and the Matrix-C particles.
<Embodiment 49> The method according to any one of Embodiments <44> to <48>, wherein administering the tumor antigen and the saponin matrix composition to the subject induces an increased production of IFN-γ by tumor antigen-specific CD8 T cells compared to administering a vaccine composition comprising the tumor antigen and a saponin matrix composition having a weight ratio of 85:15 of the Matrix-A particles to the Matrix-C particles under comparable dosing and administration conditions.
<Embodiment 50> The method according to any one of Embodiments <44> to <48>, wherein administering the tumor antigen and the saponin matrix composition to the subject elicits increased cross-presentation of the tumor antigen by antigen-presenting cells compared to administering a vaccine composition comprising the tumor antigen and a saponin matrix composition having a weight ratio of 85:15 of the Matrix-A particles to the Matrix-C particles under comparable dosing and administration conditions.
<Embodiment 51> The method according to any one of Embodiments <44> to <48>, wherein administering the tumor antigen and the saponin matrix composition to the subject results in increased activation of antigen-presenting cells compared to administering a vaccine composition comprising the tumor antigen and a saponin matrix composition having a weight ratio of 85:15 of the Matrix-A particles to the Matrix-C particles.
<Embodiment 52> The method according to any one of Embodiments <44> to <51>, wherein the tumor antigen and the saponin matrix composition are co-formulated in a single vaccine composition that is administered to the subject as an intramuscular injection.
<Embodiment 53> The method according to any one of Embodiments <44> to <51>, wherein the tumor antigen and the saponin matrix composition are administered to the subject in separate pharmaceutical compositions given at the same anatomical site within a time window of up to 24 hours.
<Embodiment 54> A method of inducing an adaptive immune response against a viral pathogen in a subject in need thereof, the method comprising administering to the subject an effective amount of a viral antigen and an effective amount of a saponin matrix composition of any one of Embodiments <1> to <13>, thereby inducing an adaptive immune response against the viral pathogen in the subject.
<Embodiment 55> The method according to Embodiment <54>, wherein the viral antigen is a SARS-CoV-2 S protein antigen.
<Embodiment 56> The method according to Embodiment <54>, wherein the viral antigen is selected from a parainfluenza virus type 3 antigen, a respiratory syncytial virus F0 antigen, and a human metapneumovirus F antigen.
<Embodiment 57> The method according to any one of Embodiments <54> to <56>, wherein the viral antigen is present at a fixed weight ratio from 0.1 μg to 200 μg of the viral antigen per 400 μg to 20 μg of the total weight of the Matrix-A particles and the Matrix-C particles.
<Embodiment 58> The method according to any one of Embodiments <54> to <56>, wherein the viral antigen is present at a fixed weight ratio from 5 μg to 100 μg of the viral antigen per 400 μg to 50 μg of the total weight of the Matrix-A particles and the Matrix-C particles.
<Embodiment 59> The method according to any one of Embodiments <54> to <58>, wherein administering the viral antigen and the saponin matrix composition to the subject induces an increased production of IFN-γ by antigen-specific CD8 T cells compared to administering a vaccine composition comprising the viral antigen and a saponin matrix composition having a weight ratio of 85:15 of the Matrix-A particles to the Matrix-C particles under comparable dosing and administration conditions.
<Embodiment 60> The method according to any one of Embodiments <54> to <59>, wherein administering the viral antigen and the saponin matrix composition to the subject elicits increased antigen cross-presentation by antigen-presenting cells compared to administering a vaccine composition comprising the viral antigen and a saponin matrix composition having a weight ratio of 85:15 of the Matrix-A particles to the Matrix-C particles under comparable dosing and administration conditions.
<Embodiment 61> The method according to any one of Embodiments <54> to <59>, wherein administering the viral antigen and the saponin matrix composition to the subject results in increased activation of antigen-presenting cells compared to administering a vaccine composition comprising the viral antigen and a saponin matrix composition having a weight ratio of 85:15 of the Matrix-A particles to the Matrix-C particles under comparable dosing and administration conditions.
<Embodiment 62> The method according to any one of Embodiments <54> to <61>, wherein the viral antigen and the saponin matrix composition are co-formulated in a single vaccine composition that is administered to the subject as an intramuscular injection.
<Embodiment 63> The method according to any one of Embodiments <54> to <61>, wherein the viral antigen and the saponin matrix composition are administered to the subject in separate pharmaceutical compositions given at the same anatomical site within a time window of up to 24 hours.
<Embodiment 64> The method according to any one of Embodiments <54> to <63>, for inducing protective immunity against the viral pathogen from which the viral antigen is derived.
<Embodiment 65> A method for eliciting cross-presentation of an antigen in a subject in need thereof, the method comprising administering to the subject an effective amount of a saponin matrix composition comprising Matrix-C particles but not Matrix-A particles and an effective amount of the antigen, thereby eliciting cross-presentation of the antigen in the subject.
<Embodiment 66> The method according to Embodiment <65>, wherein the cross-presentation of the antigen in the subject is increased compared to a method in which a saponin matrix composition comprising Matrix-A particles and Matrix-C particles at a weight ratio of 85:15 of the Matrix-A particles to the Matrix-C particles is used, under comparable dosing and administration conditions.
<Embodiment 67> A method of inducing an adaptive immune response in a subject, wherein the response is substantially independent of NLRP3 inflammasome activation, the method comprising administering to the subject an effective amount of an antigen and an effective amount of a saponin matrix composition of any one of Embodiments <1> to <13>, thereby inducing an adaptive immune response in the subject. General statements relating to the present disclosure
<Embodiment 68> The method according to any one of Embodiments <23> to <67>, wherein the subject is selected from a cow, horse, pig, sheep, or human.

When reference is made to a composition comprising Matrix-A but not Matrix-C, or alternatively a composition comprising Matrix-C but not Matrix A, the specific amounts or ratios stated herein as they relate to both Matrix-A and Matrix-C in combination shall be held to also relate to Matrix-A or Matrix-C individually.

The arrangement of the present disclosure into sections with headings and subheadings is merely to improve legibility and is not to be interpreted as limiting in any way, in particular, the division does not in any way preclude or limit combining features under different headings and subheadings with each other.

### Examples

We have determined that antigen and Matrix-M adjuvant, Matrix-A particles, and Matrix-C particles co-localize in lysosomes following uptake by bone marrow-derived dendritic cells (BMDCs). Matrix-M adjuvant, Matrix-A particles, and Matrix-C particles induce lysosomal membrane permeabilization (LMP), with Matrix-C particles showing the highest LMP potential. LMP is required for IL-1β and IL-18 secretion in vitro, of which IL-1B secretion is dependent on activation of the NLRP3 inflammasome. However, the NRLP3 inflammasome is not required for adjuvanticity of Matrix-M adjuvant in mice in vivo. As another downstream effect of LMP, Matrix-M adjuvant as well as Matrix-A particles and Matrix-C particles enable antigen cross-presentation. Thus, Matrix-induced LMP is believed to contribute to the capability of Matrix-M adjuvanted protein vaccines to induce CD8⁺ T cell responses.

### Adjuvants and Antigens

Matrix-M, Matrix-A, and Matrix-C were obtained from Novavax AB. To enable cellular tracking, Matrix-A and Matrix-C were fluorescently labeled with BODIPY (4,4-difluoro-4-bora-3a,4a-diaza-s-indacen). The labeling process involved a HATU-mediated amide coupling reaction, coupling the BODIPY-FL-amine fluorophore (Lumiprobe GmbH) with the glucuronic acid unit within the branched trisaccharide domain of Fraction-A and Fraction-C saponins. Following synthesis, the labeled conjugates were purified using reverse-phase preparative high-performance liquid chromatography (RP-HPLC) on a C18 column and isolated as powders. The labeled particles were formulated using 90% unlabeled and 10% BODIPY-labeled Fraction-A or Fraction-C saponins for Matrix-A and Matrix-C, respectively. The final BODIPY-labeled Matrix-M consisted of 85% BODIPY-labeled Matrix-A and 15% BODIPY-labeled Matrix-C. For antigen studies, ovalbumin (OVA) protein (Endograde OVA, Lionex GmbH), the SIINFEKL peptide of OVA (Iba Lifesciences), and SARS-CoV-2 recombinant spike (rS) protein (BV2373, Novavax Inc.), which is formulated as a nanoparticle, were used. For colocalization experiments, the SARS-CoV-2 rS was conjugated with Alexa Fluor™ 647 (Thermo Fisher Scientific) using NHS ester chemistry, followed by purification using Pierce™ dye removal columns (Thermo Fisher Scientific).

### Mice

Female BALB/c, C57BL/6, B6.129S6-Nlrp3^{tm/Bhk}/J mice (referred to as Nlrp3 knockout (KO) hereafter), and OT-I mice were obtained from Charles River Laboratories. All animal experiments were approved by the local animal experimentation ethical review committee (Uppsala djurförsöksetiska nämnd, approval number 5.8.18-12107/2021).

### Immunization

Mice (BALB/c mice for the Matrix dose titration; C57BL/6 and Nlrp3 KO mice for the inflammasome study), 6-10 weeks old, were immunized subcutaneously (s.c.) at the base of tail at day 0 and day 21 with 0.1 μg SARS-CoV-2 rS protein, either alone or adjuvanted with Matrix-M, Matrix-A, or Matrix-C in a total volume of 100 μL. Serum samples were collected at days 20 and 28, and additionally, at day 13 for C57BL/6 and Nlrp3 KO mice.

### BMDC Generation and Cell Culture

Primary BMDCs were generated by culturing total bone marrow cells (BMCs) isolated from tibia and femur of C57BL/6 and Nlrp3 KO mice (1-3 mice/culture) in complete Roswell Park Memorial Institute (RPMI)-1640 medium (containing 10% heat inactivated fetal bovine serum, 2 mM L-glutamine, 100 U/ml penicillin and streptomycin, and 0.05 mM 2-mercaptoethanol) supplemented with 20 ng/ml recombinant murine granulocyte-macrophage colony-stimulating factor (GM-CSF) for 7 days. A total of 4×10⁶ BMCs were seeded per Petri dish, with half the medium replaced at days 3 and 6 with fresh medium containing GM-CSF (40 ng/ml). At day 7, non-adherent and loosely adherent cells were harvested by gentle PBS washing.

B3Z cells (a gift from Professor Sebastian Springer, Constructor University, Bremen, Germany) were maintained in complete RPMI-1640 medium supplemented with 0.5 mg/mL hygromycin B.

### Spinning-Disk Confocal Microscopy

For spinning-disk confocal live-cell imaging, BMDCs (7.5×10⁵ cells/well) were seeded into sterile 8-well chambered cover glass (Cellvis) in phenol-red free complete RPMI-1640 medium supplemented with 20 ng/mL GM-CSF. On the following day, cells were stained with 50 nM LysoTracker™ Red DND-99 (Life Technologies) for 20 min and washed, and 10 pre-bleaching and background corrected images were immediately acquired at 3-min intervals. After this initial image recording, cells were exposed to BODIPY-labeled Matrix-M, Matrix-A, or Matrix-C, with or without the Alexa Fluor 647-labeled SARS-COV2 rS antigen, and images were captured at 3-min intervals for an additional 120 min in DIC (transmitted light), GFP (Ex Lumencor 475/34x, Em Chroma ET510/20m), LysoTracker Red (Ex Lumencor 555/28x, Em Chroma ET600/50m) and Alexa Fluor 647 (Ex Lumencor 648/20x, Em Chroma 89402m) channels confocally, with a Spectra-X light engine (Lumencor) providing excitation light. A Prime™ 95B 25 mm camera (Photometrics) coupled to an X-Light V2 spinning disk module (Crest Optics) on an ECLIPSE Ti2 microscope (Nikon) using a 100x/1.42 NA Plan Apochromat objective (Nikon) was used for imaging, pixel size 113 nm. During time-lapse imaging, the microscope chamber was maintained at 37 °C in a 5% CO₂ moisturized atmosphere.

To visualize LMP, BMDCs (1.25×10⁵ cells/well) were seeded as described above and subsequently loaded with 50 μg/mL Alexa Fluor 488-conjugated 3-kDa dextran and 50 μg/mL tetramethylrhodamine-conjugated 40-kDa dextran (Molecular Probes) overnight. After washing, cells were chased in dextran-free medium for 2-5 h to label lysosomes and incubated with or without 30 nM Bafilomycin A1 (BafA1, InvivoGen) for 1 hour. Cells were then imaged before and after treatment with Matrix-M, Matrix-A, or Matrix-C at 3-min intervals for up to 5 h to monitor the release of dextran from lysosomes to cytosol.

### Image Analysis

The time-lapse movies were preprocessed by concatenating the pre- and post-treatment image series from individual positions, followed by subtraction of the background fluorescence from each fluorescence channel. For Matrix and antigen channels, backgrounds were generated by median projection of the first 10 pre-bleaching frames from each position. The LysoTracker Red background was generated by median projection across all positions and timepoints for each individual experiment. Pearson's correlation coefficients (PCCs) for each time point and channel combination were calculated using the background-subtracted full frames by a Python (3.11.4) script using the scikit-image (0.22.0) and numpy (1.26.0) packages. The csv files output from the analysis script were further processed in R (3.5.1) and dplyr (1.0.5) and visualized with ggplot2 (3.3.3).

### Assessment of LMP by Microscopy

One technical replicate (of three) from each experiment was analyzed. Prior to scoring all movies were pseudonymized and hence analysis was performed on blinded data to avoid scoring bias. Scoring was done manually using the point selection tool in ImageJ. Constant and global contrast settings were used, and cells were marked when diffuse dextran fluorescence was first detectable in the cytosol. All channels were independently analyzed in random order. The transmitted light channel (DIC) analysis comprised marking of when cells underwent lysis. All cells present in a given field of view were included in the analysis.

### Assessment of LMP by Flow Cytometry

BMDCs (1.5×10⁵ cells/well) were seeded in 96-well plates in complete RPMI-1640 medium supplemented with 20 ng/mL GM-CSF, and treated with serial dilutions of Matrix-M, Matrix-A, Matrix-C or medium for 1 h. Cells were then washed and resuspended using serum-free RPMI-1640 medium followed by staining with 50 nM LysoTracker™ Deep Red (Molecular Probes) and BD Horizon™ Fixable Viability Stain 520 (FVS520; BD Biosciences) for a total of 15 and 7 min, respectively. After washing, stained cells were detached using StemPro™ Accutase™ Cell Dissociation Reagent (Gibco) and analyzed on a BD FACSCelesta™ flow cytometer (BD Biosciences). Data were processed using FlowJo software version 10.7.1 (BD Life Sciences). As positive control for reduced LysoTracker staining, cells treated with BafA1 for 1 h or with L-Leucyl-L-Leucine methyl ester (LLOMe, Merck) for 15 min, were analyzed in parallel. Normalized geometric means of fluorescence intensity (gMFI) for LysoTracker were calculated relative to untreated controls.

### Assessment of Inflammasome Activation, Cell Viability and Cytokine Production

BMDCs were seeded in 96-well plates for viability assays (0.5×10⁵ cells/well) and for cytokine analysis by ELISA (1×10⁵ cells/well). Cells were either left unprimed or primed with 0.1 μg/mL LPS (Merck) for 1 h, followed by treatment with MCC950, BafA1, Ac-YVAD-cmk (all from InvivoGen), CA074-Me (Selleck Chemicals), and disulfiram or necrosulfamide (Tocris Bioscience) for an additional 1 hour. Cells were then exposed to serial dilutions of Matrix-M, Matrix-A, or Matrix-C. After 5 or 24 h of incubation with Matrix, cell viability was assessed, and supernatants were harvested and immediately frozen for subsequent cytokine analysis by ELISA. All treatments were performed in duplicate wells. Cell viability was determined using WST-1 cell proliferation reagent (Roche), and supernatants were analyzed for IL-1β and IL-18 using mouse IL-1β and IL-18 ELISA kits according to the manufacturer's instructions (DuoSet ELISA, R&D Systems).

### Antigen Cross-Presentation and BMDC Phenotyping In Vitro

BMDCs (1.5×10⁵ cells/well) were seeded in non-treated 96-well round-bottom plates and incubated for 24 h with 0.8 mg/mL OVA (EndoGrade® Ovalbumin, Lionex GmbH) and Matrix-M, Matrix-A, or Matrix-C. For flow cytometry analysis, cells were stained with FVS780 (BD Biosciences). After washing, cells were detached with PBS-EDTA (Alfa Aesar, Thermo Fisher Scientific), resuspended in FBS-containing stain buffer (BD Biosciences), incubated with an anti-mouse CD16/CD32 antibody (2.4G2), followed by staining with F4/80:BV605 (T45-2342), CD11c:BV650 (HL3), I-E/I-E (MHCII):BV786 (M5/114.15.2), CD86:PE (GL1), CD11b:PerCP-Cy5.5 (M1/70), Ly6C:R718 (AL21, all BD Biosciences), and SIINFEKL-H-2kB (25-D1.16; eBioscience) for 20 min at 4 °C in the dark. Stained cells were washed and immediately analyzed on a BD FACSCelesta flow cytometer with data analysis performed using FlowJo software version 10.9.0.

### B3Z Assay

BMDCs (8×10⁴ cells/well) were seeded in 96-well flat-bottom cell culture plates in the presence or absence of BafA1 or epoxomicin for 1 h and incubated with 80 μg/mL OVA and Matrix-M, Matrix-A, or Matrix-C for 5 h. As a control for cell viability or MHCI expression levels, control BMDCs (without OVA) were pulsed with 5 ng/mL SIINFEKL peptide 30 min before the incubation ended. Cells were washed twice with PBS and co-cultured with B3Z cells (8×10⁴ cells/well) for 18 h, with BafA1 or epoxomicin in treated wells. Cells were then washed twice and lysed in a PBS buffer containing 0.15 mM CPRG (Roche), 9 mM MgCl₂, 0.1 mM 2-mercaptoethanol, and 0.125% NP-40. CPRG conversion by β-galactosidase was measured after 3-5 h at 570 nm using a SpectraMax® M3 spectrophotometer (Molecular Devices).

### Antigen Cross-Presentation Ex Vivo

Female C57BL/6 mice (8-10 weeks) were immunized with 50 μL intramuscular (i.m.) injection of OVA, either alone or adjuvanted with Matrix-M, into the right hind leg (musculus quadriceps femoris). The draining iliac lymph nodes (LN) were collected 24 h post-immunization and the obtained cell suspensions were pooled (7 to 13 LN per pool) and sorted by MACS using the CD11c MicroBeads Ultrapure (Miltenyi Biotec) to isolate CD11c⁺ dendritic cells (DCs). These CD11c⁺ DCs were co-cultured at a 1:4 ratio in a 96-well U-bottom cell culture plate with naïve CD8⁺ T cells isolated from spleens of OT-I mice using the Naïve CD8a⁺ T cell Isolation Kit (Miltenyi Biotec). OT-I cells had been pre-labeled with CellTrace™ Violet (CTV, Life technologies) to follow their proliferation. After three days of co-culture, cells were stained with FVS780, followed by incubation with anti-mouse CD16/CD32 antibody (2.4G2; Fc block) and antibodies targeting CD4:PerCyP5.5 (RM4-5), CD8a:BV650 (53-6.7), CD44:BV786 (IM7), and CD3e:FITC (145-2C11, all antibodies from BD Biosciences) for 30 min at 4 °C in the dark. Stained cells were washed and immediately analyzed on a BD FACSCelesta flow cytometer with data analysis performed using FlowJo software version 10.9.0. The MFI of CD44:BV786 for gated CD8⁺ T cells was used to assess their activation, and CTV dilution was analyzed to determine the percentage of divided CD8+ T cells using FlowJo's proliferation modeling tool, annotating the “generation 0” peak for undivided cells and number of observed peaks.

### ELISA for Anti-rS IgG1 and IgG2a/IgG2c Serum Antibodies

Quantification of anti-rS IgG1 and IgG2a or IgG2c serum antibodies (BALB/c or C57BL/6 background, respectively) was performed using ELISA. Serum samples were serially diluted 5-fold in eight steps, starting at 1:15 or 1:50 (days 13 and 20), 1:500, 1:5000, or 1:10,000 (day 28) for IgG1, and 1:15 (day 20), 1:15, 1:100, or 1:500 (day 28) for IgG2a, and 1:15 or 1:50 (days 13 and 20), 1:15, 1:50, 1:100, or 1:500 (day 28) for IgG2c. Absorbance was measured at 450 nm (SpectraMax M3, Molecular Devices) and anti-rS titers were calculated using a four-parameter logistic equation (SoftMax® Pro software v.7.1.1.). Pooled sera from untreated or from SARS-CoV-2 rS (adjuvanted with Matrix-M) immunized mice were used as negative and positive controls, respectively.

ELISA for hACE2 Receptor-Blocking Antibodies

Quantification of hACE2 receptor-blocking antibody titers was performed in an ELISA assay. Mouse sera were serially diluted 2-fold (starting with a 1:20 dilution). Absorbance was measured at 450 nm (SpectraMax M3, Molecular Devices). Serum antibody titers at 50% inhibition (IC50) of hACE2 to SARS-CoV-2 rS protein were then determined using the SoftMax Pro software v.7.1.1.

### Cellular Responses Using FluroSpot Assay

Antigen-specific recall cellular responses were evaluated at day 28 via the FluoroSpot assay. In short, single-cell suspensions were seeded on FluoroSpot filter plates (Mabtech) precoated with anti-IL-2, anti-IFN-γ, or anti-IL-4 capture antibodies. Cells were stimulated with 1 μg/well SARS-CoV-2 rS protein and after incubation cytokine-positive spots were developed according to the manufacturer's instructions and counted using an iSpot FluoroSpot Reader Spectrum (Autoimmune Diagnostika GmbH). The number of IFN-γ- and/or IL-2-, or IL-4-secreting cells was obtained by subtracting the mean background number of the respective medium control for each mouse, followed by averaging triplicate wells. The data are shown as geometric mean (GM) for each group. The results are expressed as the number of cytokine-secreting cells per 10⁶ splenocytes.

### T-Cell Responses Using Intracellular Cytokine Staining (ICS)

For ICS, splenocytes (1×10⁶ cells/well) were cultured in U-bottom 96-well cell culture plates and stimulated with 5 μg/mL SARS-CoV-2 rS overnight. Next, plates were incubated with GolgiStop™ and GolgiPlug™ (BD Biosciences) for 5 hours. Cells were stained with FVS780, washed and incubated with an anti-mouse CD16/CD32 antibody (2.4G2) antibodies targeting CD3:R718 (clone 145-2C11), CD4:PerCpCy5.5 (RM4-5), CD8a:BV650 (53-6.7), and CD44:BV786 (IM7) (BD Biosciences). Cells were incubated with BD Cytofix/Cytoperm™ Solution (BD Biosciences) for 20 min at 4 °C in the dark and kept in PBS overnight after washing. The next day, cells were incubated with Perm/Wash™ Buffer (BD Biosciences) for 15 min at room temperature and stained with IFN-γ:BV421 (clone XMG1.2), TNF:PE (MP6-XT22), IL-2:PE-Cy7 (JES6-5H4), and IL-4:APC (11B11) (BD Biosciences). After washing, stained cells were analyzed on a BD FACSCelesta flow cytometer and data processed using FlowJo software version 10.9.0. Live CD3⁺ T cells were gated for CD4⁺ and CD8⁺ T cells. Within CD4⁺ and CD8⁺ T cells, the frequency of single cytokine-positive/CD44⁺ cells was determined. Frequencies of unstimulated medium controls were subtracted from rS-stimulated matched samples for background reduction.

### Statistical Analysis

Specific information on sample sizes, number of experiments, technical replicates and statistical analyses is provided in the figure legends. p-values of less than 0.05 were considered statistically significant (*p≤0.05; **p≤0.01; ***p≤0.001; ****p≤0.0001). The graphs were prepared, and statistics calculated using GraphPad Prism® version 9.4.0 or later (GraphPad Software).

### Matrix-M Accumulation Inside Lysosomes

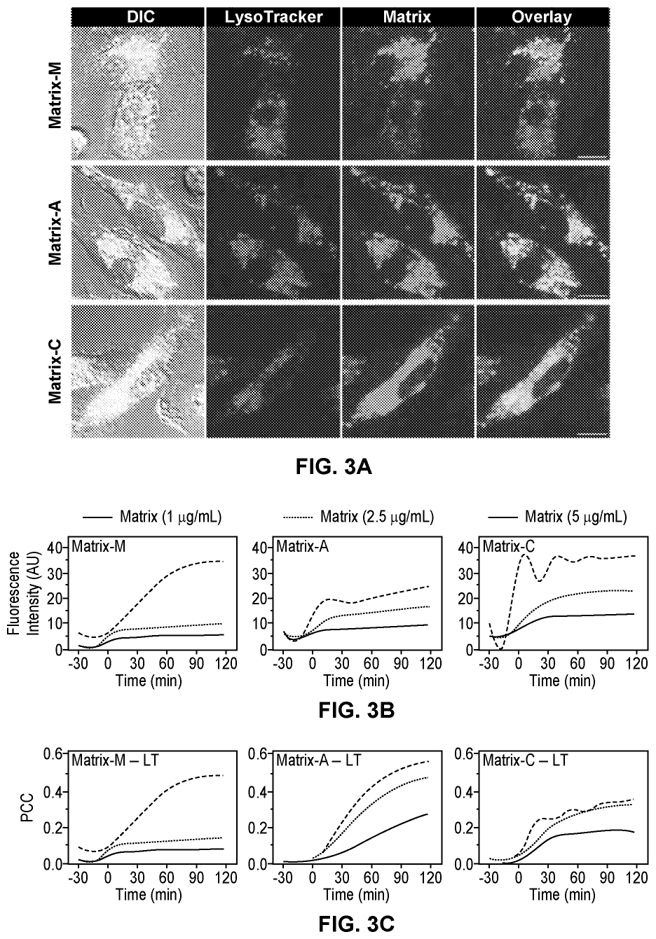

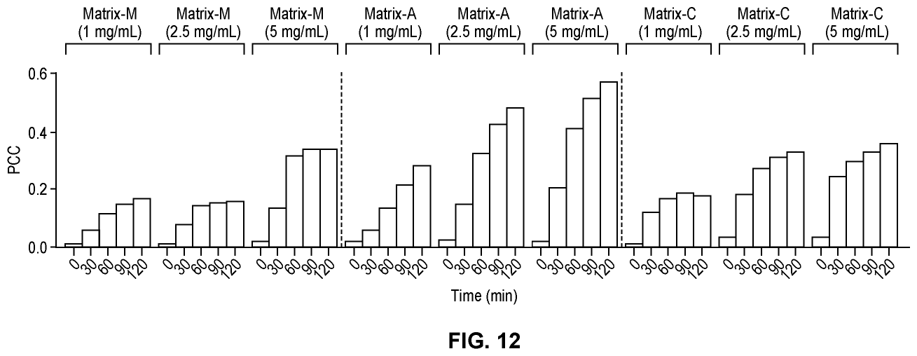

In order to determine the intracellular localization of Matrix-M after cellular uptake, we employed both BODIPY-labeled Matrix-M and LysoTracker. BODIPY labeling of Matrix-A and Matrix-C particles enabled us to observe the trafficking of Matrix-M within the cells, while LysoTracker, which selectively accumulates in acidic organelles such as lysosomes upon protonation, allowed assessment of whether Matrix-M localizes to lysosomes through the endocytic pathway. For this, BMDCs were stained with LysoTracker, followed by incubation with BODIPY-conjugated Matrix-M. Using time-lapse spinning-disk confocal imaging, we observed that shortly after exposure, BODIPY-labeled Matrix-M became visible inside the cells (FIG. 3A) with a time- and dose-dependent increase in BODIPY fluorescent signal (FIG. 3B). Image overlays displayed a substantial overlap between LysoTracker and BODIPY signals (FIG. 3A). Furthermore, by calculating Pearson's correlation coefficient (PCC) for each frame, a marked increase in the degree of colocalization between LysoTracker and BODIPY-labeled Matrix-M was observed (FIG. 3C, FIG. 12). Similarly, cellular uptake and lysosomal localization were observed using BODIPY-labeled Matrix-A or BODIPY-labeled Matrix-C (FIGS. 3A-3C). Matrix-C displayed a higher fluorescence intensity compared to Matrix-A at the highest tested concentration (5 μg/mL) (FIG. 3B). By contrast, Matrix-A reached a higher level of colocalization with LysoTracker than Matrix-C (FIG. 3C). Altogether, these results indicate an accumulation of Matrix-M, Matrix-A, and Matrix-C within lysosomes upon cellular uptake.

Matrix-M Induces Lysosomal Membrane Permeabilization (LMP) Leading to the Release of Lysosomal Contents into the Cytosol

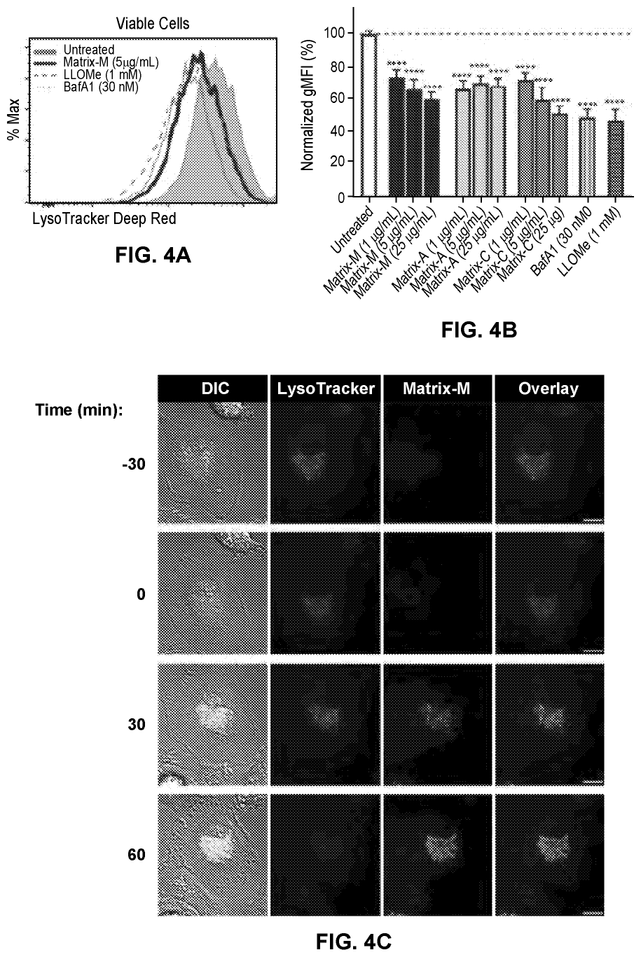

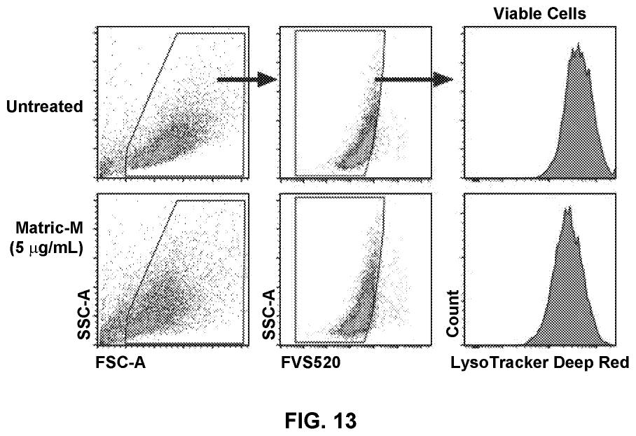

To evaluate the effect of Matrix-M on lysosomal membrane integrity, which is required to maintain a low intra-lysosomal pH, we assessed lysosomal acidification by monitoring LysoTracker cellular staining using flow cytometry. In viable BMDCs (gating strategy shown in FIG. 13), exposure to Matrix-M adjuvant resulted in a reduction of LysoTracker staining (FIGS. 4A, 4B). Similar reductions in LysoTracker staining were observed following exposure to Matrix-A and Matrix-C, indicating that all three Matrix adjuvants disrupt the pH gradient across the lysosomal membrane, or disrupt the lysosomal membranes themselves. As expected, the positive controls, BafA1 and LLOMe, also reduced LysoTracker staining (FIGS. 4A, 4B). BafA1 and LLOMe disrupt lysosomal acidification via distinct mechanisms. BafA1 inhibits the vacuolar ATPase (V-ATPase) proton pump responsible for maintaining low lysosomal pH, whereas LLOMe induces LMP by forming membrane pores. Visible Matrix-M-induced reduction of LysoTracker signal could also be noted among single cells imaged by spinning-disk confocal live-cell microscopy (FIG. 4C).

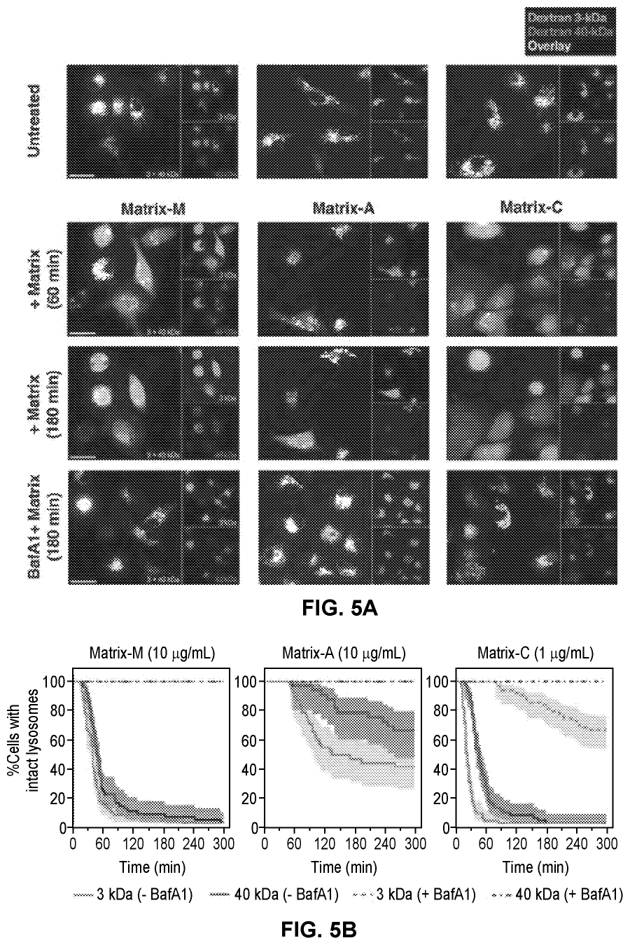

We next sought to investigate whether the Matrix-induced loss of lysosomal acidification could be attributed to pore-forming activity of Matrix-M, leading to LMP. To this end, we monitored induction of LMP by tracking the translocation of fluorescent dextran particles (3- and 40-kDa) from lysosomes to the cytosol using time-lapse spinning-disk confocal imaging³⁴. Before the addition of Matrix-M, cells had intact lysosomes as indicated by the punctate morphology of the fluorescent dextran particles (FIG. 5A). However, a diffuse cytosolic staining pattern for both 3- and 40-kDa dextran particles appeared in the cells upon Matrix-M treatment, demonstrating the induction of pores permeable for particles up to 40-kDa size (FIG. 5A). Similarly, the treatment of dextran-loaded BMDCs with Matrix-A or Matrix-C resulted in the release of lysosomal content into the cytosol (FIG. 5A). Of note, Matrix-M and Matrix-C were found to be more potent than Matrix-A in promoting LMP and dextran release into the cytosol (FIG. 5B, note different concentrations of Matrix adjuvants). Given that lysosomal hydrolytic enzymes depend on acidic pH for optimal activity, we next assessed whether lysosomal acidification is required for the LMP-inducing effect of Matrix adjuvants. For this, BMDCs preloaded with dextran particles were incubated with the lysosomal acidification inhibitor BafA1 for 1 h before exposure to Matrix-M, Matrix-A, or Matrix-C. Notably, inhibition of lysosomal acidification completely blocked the release of both 3- and 40-kDa dextran particles from the lysosome to the cytosol in response to Matrix-M and Matrix-A (FIG. 5B). In response to Matrix-C, blockade of lysosomal acidification resulted in complete inhibition of 40-kDa dextran release, but only partial inhibition of 3-kDa dextran release into the cytosol (FIG. 5B). These results demonstrate that all three Matrix adjuvants induce LMP, albeit with distinct potencies, as Matrix-C and Matrix-M exhibited stronger LMP-inducing effects compared to Matrix-A.

### Matrix-M Induces Inflammasome Activation and IL-1β Secretion

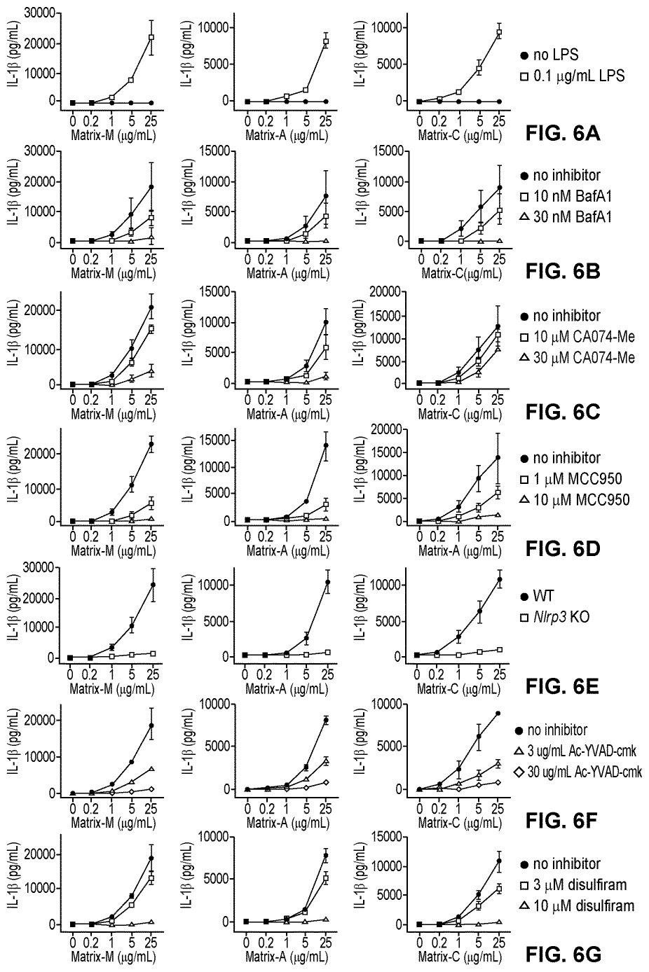

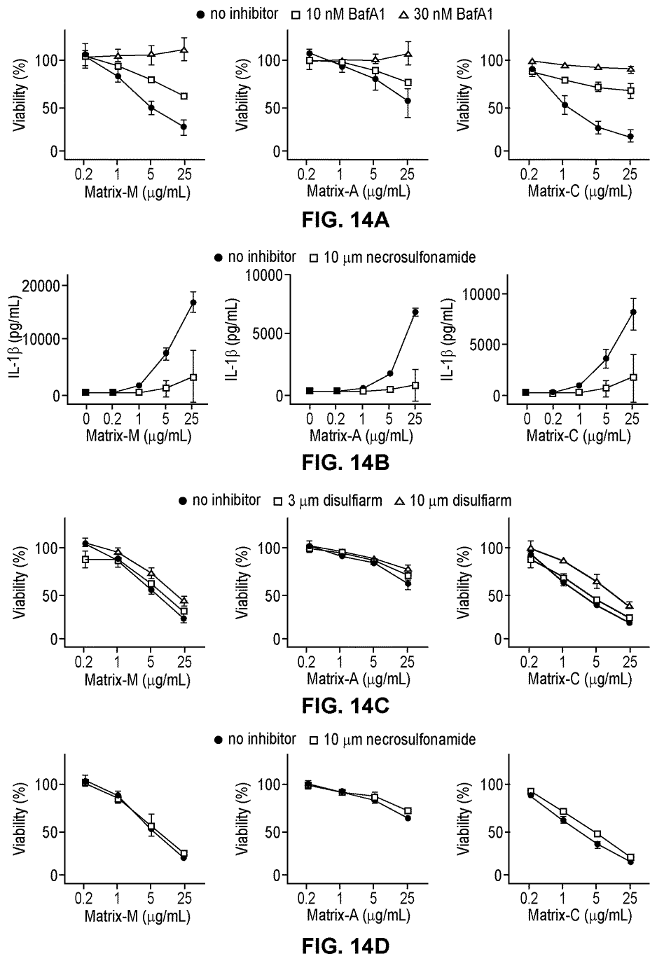

Release of lysosomal contents into the cytosol following LMP can initiate inflammasome activation, a process essential for converting pro-IL-1β into its active form and the subsequent release of biologically active IL-1β. To assess the ability of Matrix-M to induce inflammasome activation, non-primed and LPS-primed BMDCs were treated with increasing doses of Matrix-M for 5 h, and released IL-1β levels were measured in supernatants by ELISA. Matrix-M induced the release of IL-1β from LPS-primed BMDCs, with higher doses of Matrix-M resulting in greater IL-1β secretion (FIG. 6A). Similarly, a dose-dependent secretion of IL-1β was observed following exposure of the cells to Matrix-A or Matrix-C. Notably, Matrix-M induced higher levels of IL-1β than either Matrix-A or Matrix-C. By contrast, non-LPS primed BMDCs did not release IL-1β upon treatment with any of the Matrix adjuvants. To investigate whether lysosomal acidification and thereby LMP is required for Matrix-induced IL-1β secretion, LPS-primed BMDCs were pretreated with increasing doses of lysosomal acidification inhibitor BafA1 before Matrix adjuvant treatment. BafA1 blocked the release of IL-1β induced by Matrix-M, Matrix-A, and Matrix-C in a dose-dependent manner (FIG. 6B), indicating that lysosomal acidification is crucial for Matrix-induced IL-1β secretion and suggesting a link to LMP. Of note, blockade of lysosomal acidification also prevented the reduction in cell viability observed in response to Matrix adjuvants (FIG. 14A).

One key lysosomal mediator known to have inflammasome activating capability after its release into the cytosol is cathepsin B (CatB). To test whether CatB contributes to Matrix-induced IL-1β release, LPS-primed BMDCs were pre-treated with the CatB inhibitor CA074-Me before Matrix-M, Matrix-A, or Matrix-C treatment. Inhibition of CatB substantially lowered IL-1β levels induced by Matrix-M and Matrix-A, whereas Matrix-C-induced IL-1β was only marginally affected (FIG. 6C).

Among the various inflammasomes, the NLRP3 inflammasome is of particular interest, as lysosomal damage is one of its known activating triggers. To further investigate whether Matrix-induced IL-1β release occurs through NLRP3 inflammasome activation, LPS-primed BMDCs were pretreated with MCC950, an inhibitor of NLRP3 inflammasome assembly. This effectively blocked Matrix-induced IL-1β release (FIG. 6D). In line with this, NLRP3-deficient BMDCs failed to release IL-1β upon exposure to Matrix adjuvants (FIG. 6E). Given that NLRP3 inflammasome activation results in the activation of caspase-1, which processes pro-IL-1β to mature IL-1β, the dependency of IL-1β release on caspase-1 activation was tested. Pretreatment with the caspase-1 inhibitor Ac-YVAD-cmk resulted in a marked reduction of IL-1β secretion in response to Matrix-M, Matrix-A, and Matrix-C (FIG. 6F). This demonstrates that NLRP3 inflammasome activation and caspase-1 are crucial for Matrix-induced IL-1β secretion.

IL-1β lacks the signal sequence required for ER/Golgi-mediated secretion and is consequently released from cells through an unconventional secretion pathway involving gasdermin D (GSDMD) pores. To investigate the potential role of GSDMD in Matrix-induced IL-1β release, LPS-primed BMDCs were pretreated with disulfiram and necrosulfonamide (NSA), both inhibitors of GSDMD that block pore formation by preventing GSDMD oligomerization. Both inhibitors dose-dependently blocked IL-1β release induced by Matrix-M, Matrix-A, and Matrix-C (FIG. 6G, FIG. 14B), showing that Matrix-induced IL-1B is released through GSDMD pores. Inflammasome-mediated GSDMD pore formation can result in either sublytic IL-1β release without causing cell death or lytic pyroptosis. Blocking GDSMD pore formation partially mitigated the reduction in cell viability observed after exposure to Matrix adjuvants (FIGS. 14C, 14D).

### Matrix-M Induces IL-18 Release Independently of NLRP3 Inflammasome Activation

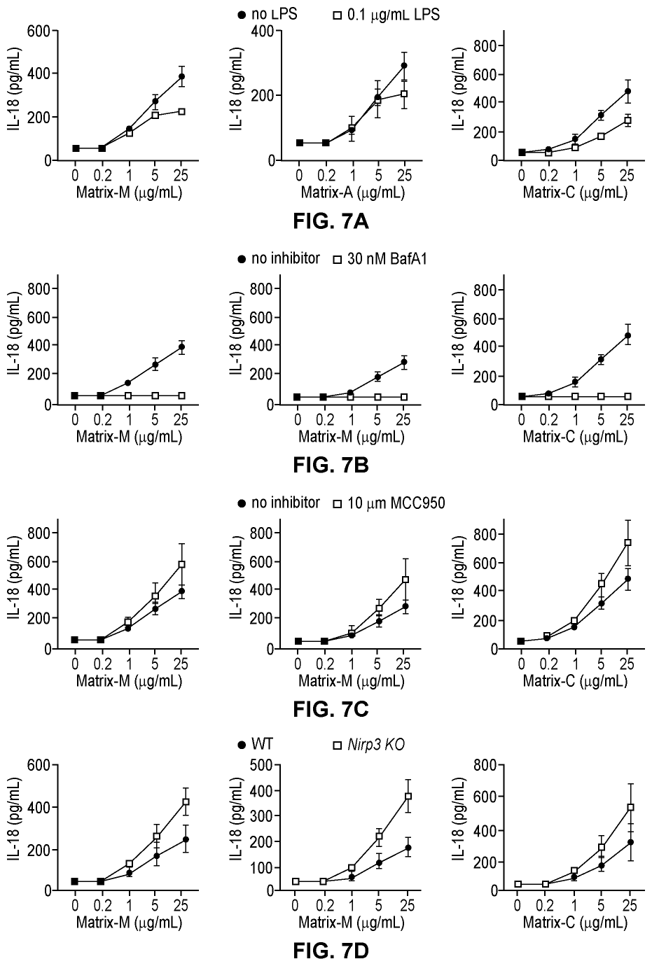

In addition to IL-1β, IL-18 can be activated through NLRP3 and other inflammasomes. To evaluate the role of Matrix-M, Matrix-A, and Matrix-C in IL-18 release, we measured the levels of IL-18 secreted by non-primed and LPS-primed BMDCs using ELISA. IL-18 release was observed in response to all three adjuvants, with non-primed cells secreting higher levels than LPS-primed cells (FIG. 7A). Inhibition of lysosomal acidification again completely blocked Matrix-induced IL-18 release, linking this process to Matrix-induced LMP (FIG. 7B). However, and in contrast to observations for IL-1β, Matrix-induced IL-18 secretion was not dependent on NLRP3 inflammasome activation. In fact, inhibition of NLRP3 with the assembly inhibitor MCC950, or the use of NLRP3-deficient BMDCs, resulted in elevated IL-18 secretion responses compared to controls (FIGS. 7C, 7D).

### NLRP3 Inflammasome Activation is not Required for Matrix-M Adjuvanticity In Vivo

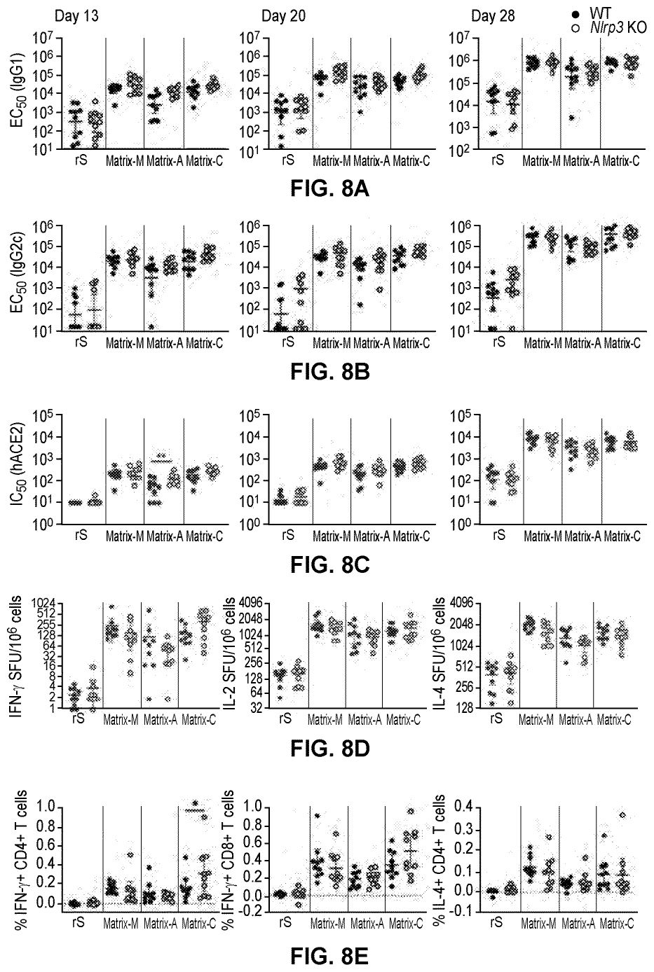

Given the observed Matrix-induced NLRP3 inflammasome activation in vitro, we investigated whether this activation was essential for the adjuvanticity of Matrix-M in vivo. Wild type (WT, C57BL/6 mice) and NLRP3-deficient mice (Nlrp3 KO mice) were immunized in a two-dose immunization regimen with SARS-CoV-2 rS antigen. At days 13 and 20 post-primary immunization as well as at day 28 (7 days post-secondary immunization), similar titers of rS-specific IgG1, IgG2c, and functional human angiotensin converting enzyme (hACE)-2 receptor-blocking antibodies were found in sera of WT and Nlrp3 KO mice, with both adjuvanted groups showing superior responses compared to their respective unadjuvanted antigen group (FIGS. 8A-8C). Comparable results were observed using Matrix-A and Matrix-C as adjuvants, although Nlrp3 KO mice displayed higher hACE2 binding inhibition titers than WT mice in the Matrix-A-adjuvanted group at day 13 (FIG. 8C).

Analysis of splenocytes producing IFN-γ, IL-2, or IL-4 following rS restimulation at day 28 showed clear adjuvant effects across Matrix-M-, Matrix-A-, and Matrix-C-adjuvanted groups, regardless of the presence or absence of the NLRP3 inflammasome (FIG. 8D). ICS analysis revealed similar frequencies of antigen-specific IFN-γ-producing CD4⁺ and CD8⁺ T cells, as well as IL-4-producing CD4⁺ T cells in WT and Nlrp3 KO mice in the non-adjuvanted and Matrix-M- and Matrix-A-adjuvanted groups (FIG. 8E). In the Matrix-C-adjuvanted groups, a higher frequency of antigen-specific IFN-γ-producing CD4⁺ T cells was observed in Nlrp3 KO mice, while similar frequencies of IFN-γ-producing CD8⁺ and IL-4-producing CD4⁺ T cells were found in Matrix-C-adjuvanted WT and Nlrp3 KO mice. Collectively, the data demonstrate that Matrix-M, Matrix-A, and Matrix-C show potent adjuvant effects in vivo both in the presence and absence of the NLRP3 inflammasome.

### Matrix-M Enables Antigen Cross-Presentation in BMDCs

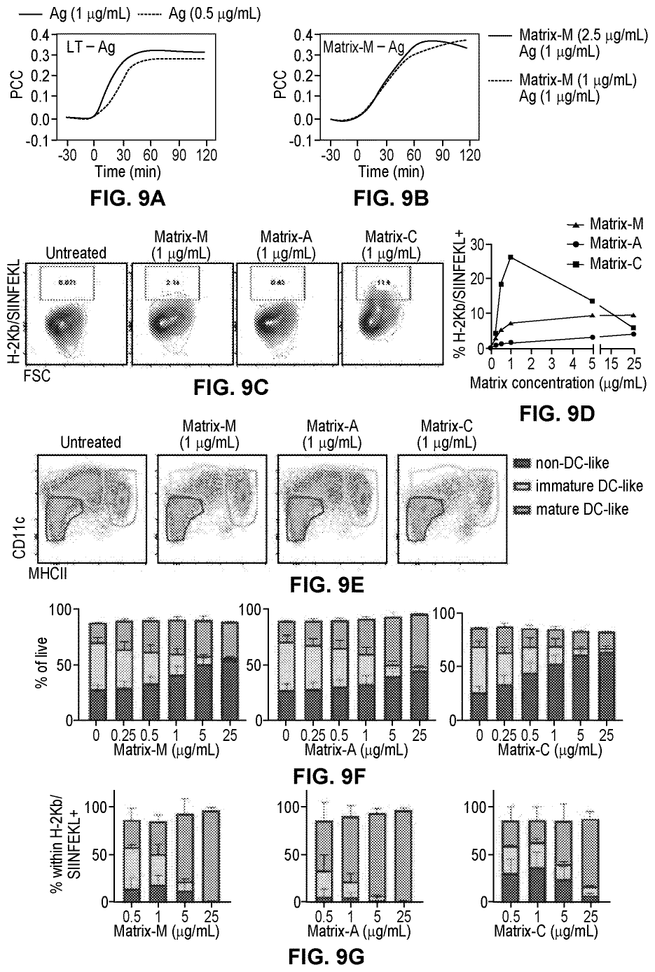

The localization of Matrix-M inside lysosomes together with its observed pore-forming activity, could allow exogenous antigens to reach the cytosol. This, in turn, may enable endocytosed antigens to enter the MHCI processing pathway, thereby promoting antigen cross-presentation. Given that Matrix-A and Matrix-C particles in Matrix-M are not physically linked to antigens and thus Matrix-M does not serve as a direct antigen delivery system, we first evaluated the colocalization of the antigen with lysosomes and Matrix-M as a prerequisite condition for cross-presentation. LysoTracker-loaded BMDCs were incubated with BODIPY-conjugated Matrix-M together with Alexa Fluor 647-labeled SARS-CoV-2 rS antigen and monitored by time-lapse spinning-disk confocal live-cell imaging. PCC analysis revealed a marked time-dependent increase in the degree of colocalization between LysoTracker and the antigen, confirming antigen internalization into the lysosomes (FIG. 9A). In parallel, colocalization between Matrix-M and the antigen was also confirmed (FIG. 9B). These results demonstrate that both Matrix-M and the antigen accumulate within lysosomes upon cellular uptake.

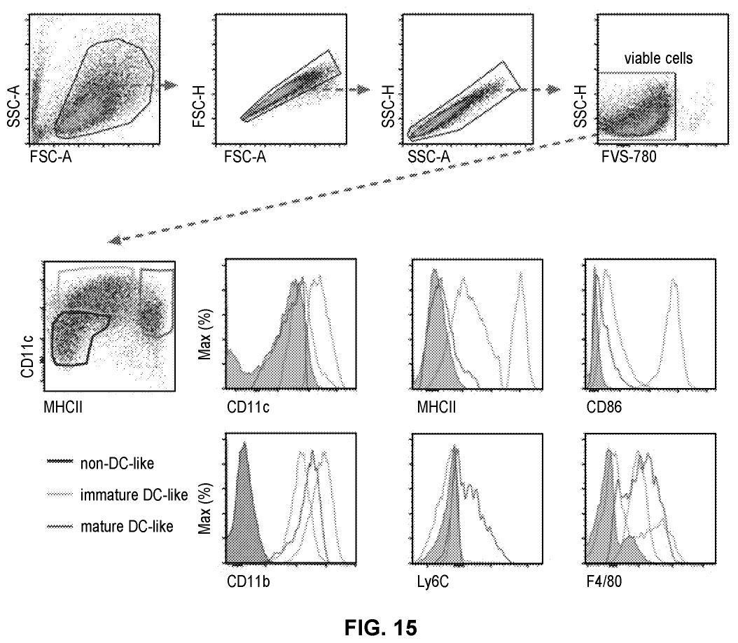

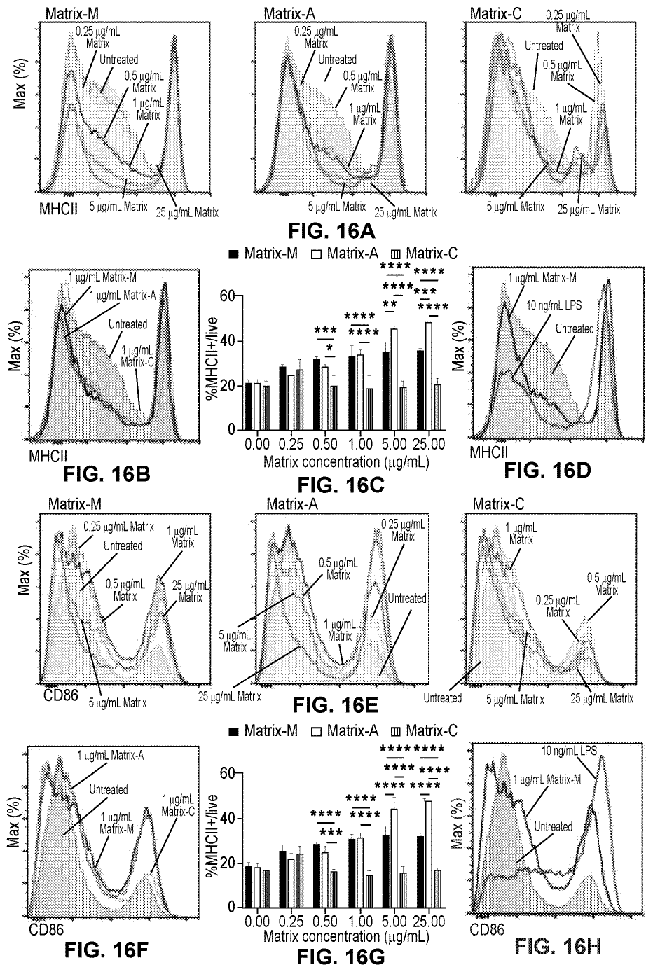

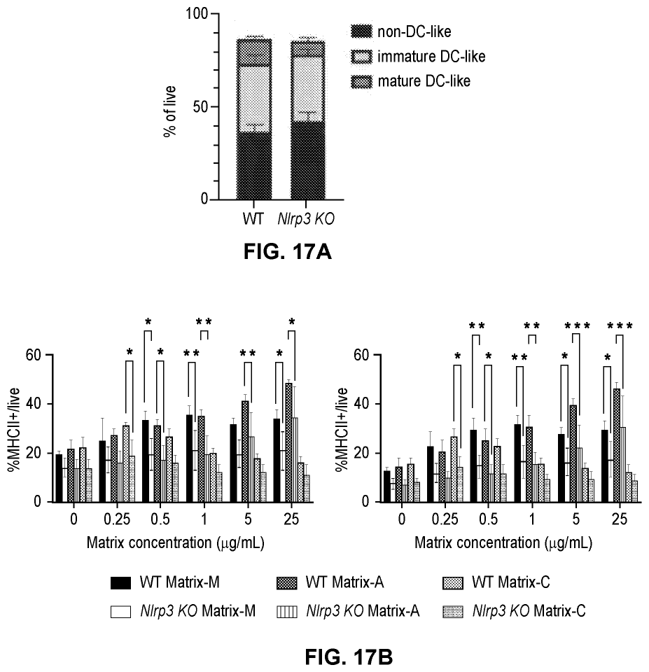

The ability of Matrix-M to induce antigen cross-presentation was subsequently investigated using the 25-D1.16 monoclonal antibody, which specifically recognizes cell surface complexes of the OVA peptide SIINFEKL bound to the MHCI molecule H-2Kb. BMDCs treated with OVA and Matrix-M for 24 h showed a Matrix-M-induced cross-presentation (FIGS. 9C, 9D). Similarly, antigen cross-presentation was observed in BMDCs after treatment with Matrix-A and Matrix-C (FIGS. 9C, 9D). When comparing the cross-presentation-inducing potential of the Matrix adjuvants, Matrix-C elicited the highest frequency of antigen cross-presenting cells at Matrix concentrations of 5 μg/mL or lower, followed by Matrix-M, while Matrix-A induced the lowest level of cross-presentation. Both Matrix-M and Matrix-A promoted a concentration-dependent increase in the frequency of antigen cross-presenting cells, whereas treatment with Matrix-C at concentrations above 5 μg/mL resulted in a reduced frequency of cross-presenting BMDCs. Phenotypic analysis of BMDCs, categorized into non-dendritic cell (DC)-like, immature DC-like, and mature DC-like populations (gating strategy shown in FIG. 15), showed a decrease in the proportion of immature DC-like cells after Matrix treatment (FIGS. 9E, 9F). Antigen cross-presentation was primarily driven by mature DC-like cells at 5 and 25 μg/mL Matrix concentrations, irrespective of the Matrix formulation (Matrix-M, Matrix-A, or Matrix-C) used (FIG. 9G). Furthermore, the effect of Matrix adjuvants on BMDC activation was investigated, with Matrix-M and Matrix-A showing the strongest effect, as indicated by increased frequencies of MHCII⁺ and CD86⁺ cells (FIGS. 16A-16H). At higher concentrations (5 and 25 μg/mL), Matrix-A treatment led to higher proportions of MHCII⁺ and CD86⁺ BMDCs compared to Matrix-M (FIGS. 16C, 16G). In line with the in vitro finding that the Matrix-induced IL-1β secretion depends on NLRP3 inflammasome activation, the upregulation of activation markers MHCII and CD86 on BMDCs was reduced in NLRP3-deficient cells following Matrix-M, Matrix-A, or Matrix-C treatment (FIGS. 17A, 17B). Altogether, these results suggest that the combination of Matrix-C and Matrix-A in Matrix-M effectively merges Matrix-C's potential for antigen cross-presentation with Matrix-A's ability to promote APC activation.

### Lysosomal Acidification and Proteasomal Degradation Contribute to Matrix-Induced Antigen Cross-Presentation

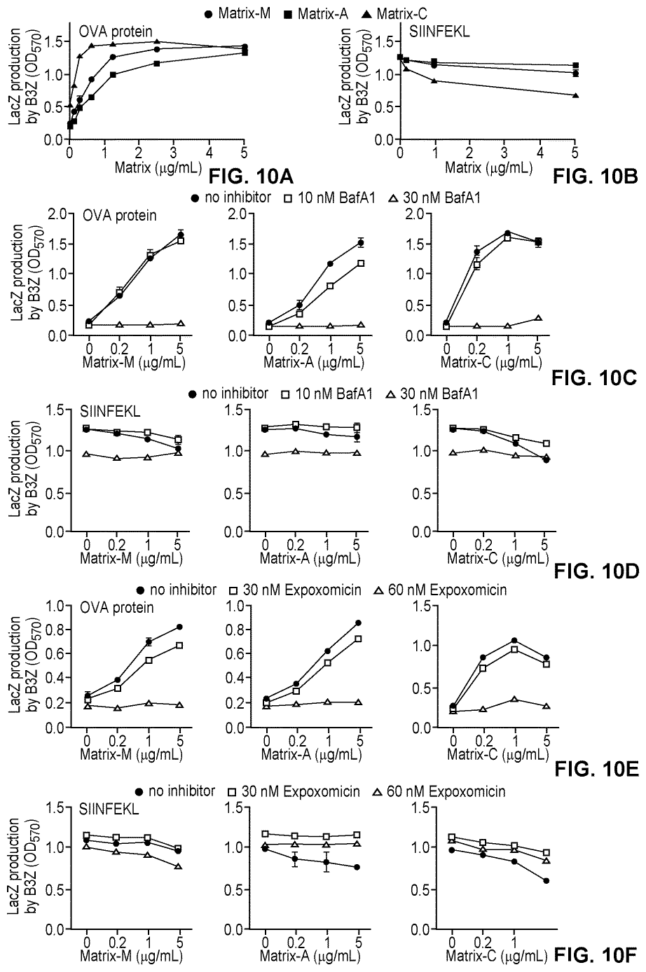

To assess the ability of APCs to cross-prime CD8⁺ T cells, BMDCs were co-cultured with B3Z cells, a co-stimulation independent reporter T-cell line. The B3Z cross-presentation assay, in which the presentation of SIINFEKL via H-2K^{b} activates T-cell receptor signaling inducing β-galactosidase expression, showed a concentration dependent induction of cross-priming, with a higher potential of Matrix-C compared to Matrix-M and Matrix-A (FIG. 10A). Importantly, the exposure of cells to Matrix-M, Matrix-A or Matrix-C did not alter MHCI presentation or cell viability, as presentation of pulsed SIINFEKL was similar or slightly reduced within the tested concentration range (FIG. 10B). Blockade of lysosomal acidification showed that Matrix-induced cross-presentation was dependent on a low lysosomal pH, suggesting a link to LMP (FIGS. 10C, 10D), consistent with the observed inhibition of dextran release by BafA1 (FIG. 5B). For the cross-presentation of exogenous antigens, two main intracellular pathways, the “endosomal” and “cytosolic,” have been proposed⁴². Given that Matrix-induced dextran release indicated antigen translocation into the cytosol, the role of proteasomal degradation, a key component of the cytosolic pathway in cross-presentation, was examined. Inhibition of proteasomal activity by epoxomicin resulted in a strong reduction in cross-presentation induced by Matrix-M, Matrix-A, and Matrix-C, confirming that Matrix-induced antigen translocation into the cytosol underlies Matrix-driven cross-presentation (FIGS. 10E, 10F).

### Matrix-M Induces Dose-Dependent CD8⁺ T-Cell Responses In Vivo

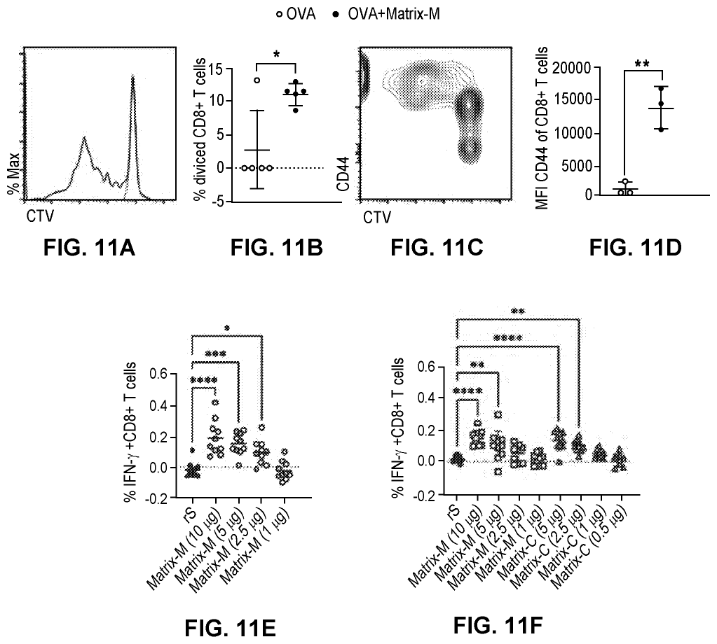

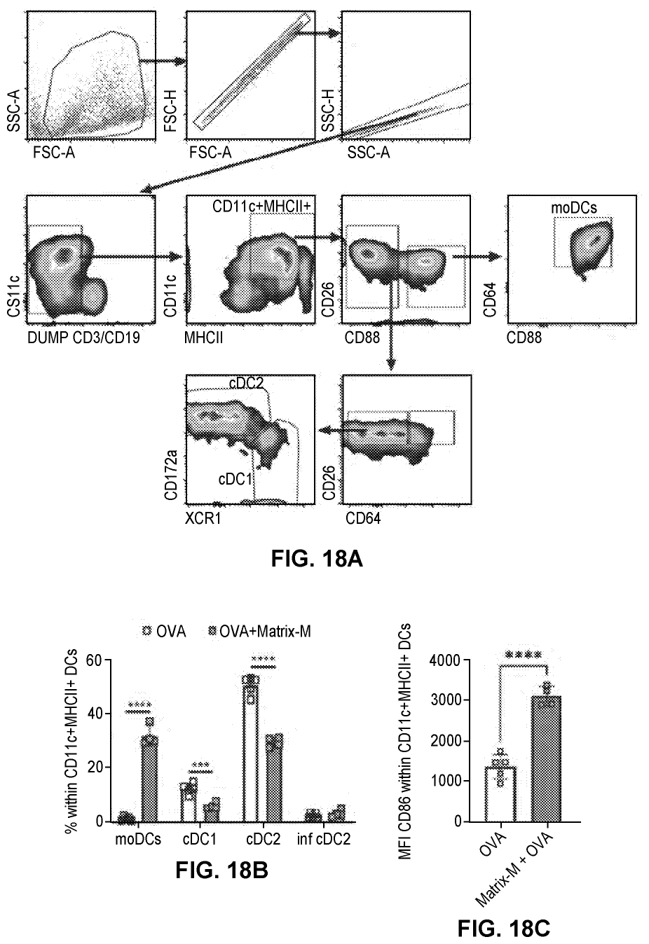

To further evaluate the ability of Matrix-M to induce antigen cross-presentation and generate antigen-specific CD8⁺ T-cell responses, two different approaches were employed. First, antigen cross-presentation was assessed ex vivo by co-culturing sorted CD11c⁺ DCs, which were loaded in vivo with OVA either in presence or absence of Matrix-M, with naïve OT-I CD8⁺ T cells that carry an OVA peptide-specific T-cell receptor. Immunization with OVA adjuvanted with Matrix-M resulted in enhanced proliferation and activation of naïve OVA-specific CD8⁺ T cells in ex vivo co-cultures (FIGS. 11A-11D). Analysis of the CD11c− sorted DCs showed a shift in proportions of DC subsets, with an increase in monocyte-derived DCs (moDCs) and a decrease in conventional DC1 and DC2 (cDC1 and cDC2) subpopulations in the group receiving Matrix-M (FIGS. 18A, 18B). Moreover, Matrix-M treatment led to increased activation of DCs in the draining lymph nodes (dLNs) (FIG. 18C).

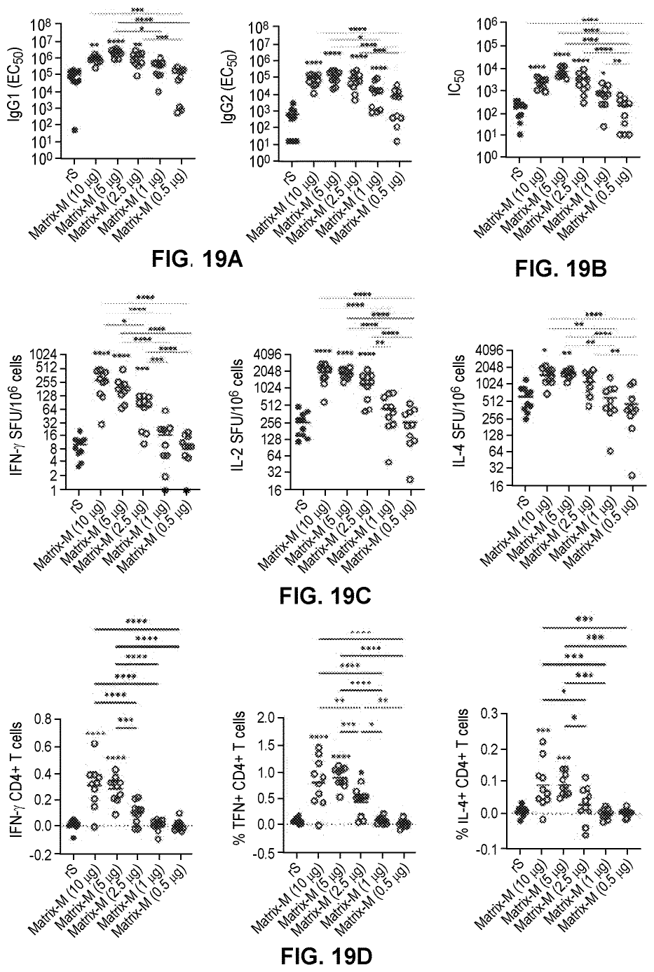

In a second approach, the effect of Matrix-M adjuvant on CD8⁺ T-cell responses was investigated in vivo using a two-dose immunization scheme with SARS-CoV-2 rS protein, where different doses of Matrix-M were administered. ICS for IFN-γ demonstrated a dose dependent induction of rS-specific CD8⁺ T-cell responses (FIG. 11E). In addition, a dose response was observed for the generation of specific serum antibodies, cellular responses, and CD4⁺ T-cell responses (FIGS. 19A-19D). Similarly, a dose-dependent induction of antigen-specific CD8⁺ T cells was observed when varying doses of Matrix-A and Matrix-C adjuvants were used for rS immunizations (FIG. 11F). Although non-adjuvanted rS immunization did not elicit detectable rS-specific CD8⁺ T cells, the addition of 2.5 μg Matrix-M and Matrix-C or 5 μg Matrix-A was sufficient to induce CD8⁺ T-cell responses. Altogether these data demonstrate the ability of Matrix-M, as well as that of Matrix-A and Matrix-C, to induce specific CD8⁺ T cells responses to exogenous antigens, as demonstrated with two distinct antigens, OVA and SARS-CoV-2 rS protein.

Our study demonstrated that DCs sorted from the dLNs after immunization with OVA and Matrix-M could activate OT-I CD8⁺ T cells ex vivo. In the Matrix-M-adjuvanted group, moDCs, absent in the dLNs of the antigen-only immunized mice, accounted for approximately one-third of the CD11c⁺/MHCII⁺ DC population. In alignment with our in vitro and ex vivo findings, SARS-CoV-2 rS vaccines adjuvanted with either Matrix-M, Matrix-A, or Matrix-C elicited rS-specific CD8⁺ T-cell responses in vivo in a dose-dependent manner. Non-adjuvanted rS vaccination failed to induce rS-specific CD8⁺ T-cell responses. A minimum dose of 2.5 μg Matrix-M or Matrix-C, or 5 μg of Matrix-A was required to induce detectable antigen-specific CD8⁺ T-cell responses. This further indicates synergistic effects between Matrix-A and Matrix-C in Matrix-M. Previous investigations have demonstrated that Matrix-C and Matrix-M can generate CD8⁺ T-cell responses in mice (Bengtsson, Song et al. 2016, den Brok, Bull et al. 2016, Tian, Patel et al. 2021). Matrix-M has the potential to induce antigen cross-presentation and CD8⁺ T-cell responses in pre-clinical models. In clinical studies, the NVX-CoV2373 vaccine, containing SARS-CoV-2 rS protein and Matrix-M, has demonstrated the ability to trigger rS-specific CD8⁺ T-cell responses in 10-50% of vaccine recipients (Rydyznski Moderbacher, Kim et al. 2022, Zhang, Mateus et al. 2022). Notably, the frequencies of rS-specific memory CD8⁺ T cells were comparable to those observed in individuals who had recovered from SARS-CoV-2 infection, at the 6-month follow up. In addition to vaccine-induced anti-viral responses, CD8⁺ T cells play a crucial role in therapeutic cancer vaccines. Matrix-M may, therefore, also be applicable in areas outside infectious disease vaccines, as tested for other saponin-based adjuvants in clinical trials (NCT05638698).

Here, we demonstrate that Matrix-M and co-administered antigens enter lysosomes together, where Matrix-M then induces LMP in an acidification-dependent process, thus enabling antigen translocation into the cytosol, NLRP3 inflammasome activation, antigen cross-presentation, and the generation of antigen-specific CD8⁺ T-cell responses. In addition, even in the event of LMP, Matrix-M induces high antibody titers and antigen-specific CD4⁺ T-cell responses, indicating a balanced antigen presentation via both MHCI and MHCII pathways. The combination of Matrix-C and Matrix-A in Matrix-M thus confers a dual advantage: the potent ability of Matrix-C to enable antigen cross-presentation and the capacity of Matrix-A to strongly activate APCs.

### Adjuvant Effect of a Range of Matrix-A:Matrix-C Ratios

The adjuvant Matrix-M and its components Matrix-A, and Matrix-C have been shown to induce antigen-cross presentation that is a condition to support the generation of CD8⁺ T cell responses by a protein-based vaccine (see above).

Different ratios of Matrix-A to Matrix-C ranging from Matrix-A only to Matrix-C only were evaluated for their ability to adjuvant the generation of antigen-specific T-cell responses as analyzed in spleens. The saponin dose was kept constant at 5 μg. In addition to the T-cell response, the adjuvanticity of the different ratios on rS-specific serum responses was analyzed. The immunization study was performed in C57BL/6 mice and SARS-CoV-2 rS protein (SARS-CoV-2 rS Drug Substance/Wuhan) was used as a model antigen. The study design is outlined in 3.

TABLE 3
Treatment groups and schedule

| | | Ratio | Immuni- | Adminis- | |
|---|---|---|---|---|---|
| | | Matrix- | zation(s) | tration | No. of |
| Group | Antigen* | A:Matrix-C* | (day) | (100 μl/mouse) | Animals |
| 1 | rS | N/A | 0, 7 | Subcutaneous | 10 |
| 2 | | 100:0 | | (s.c.) | |
| 3 | | 85:15 | | | |
| 4 | | 70:30 | | | |
| 5 | | 55:45 | | | |
| 6 | | 40:60 | | | |
| 7 | | 25:75 | | | |
| 8 | | 10:90 | | | |
| 9 | | 0:100 | | | |

*1 μg rS and 5 μg saponin per dose

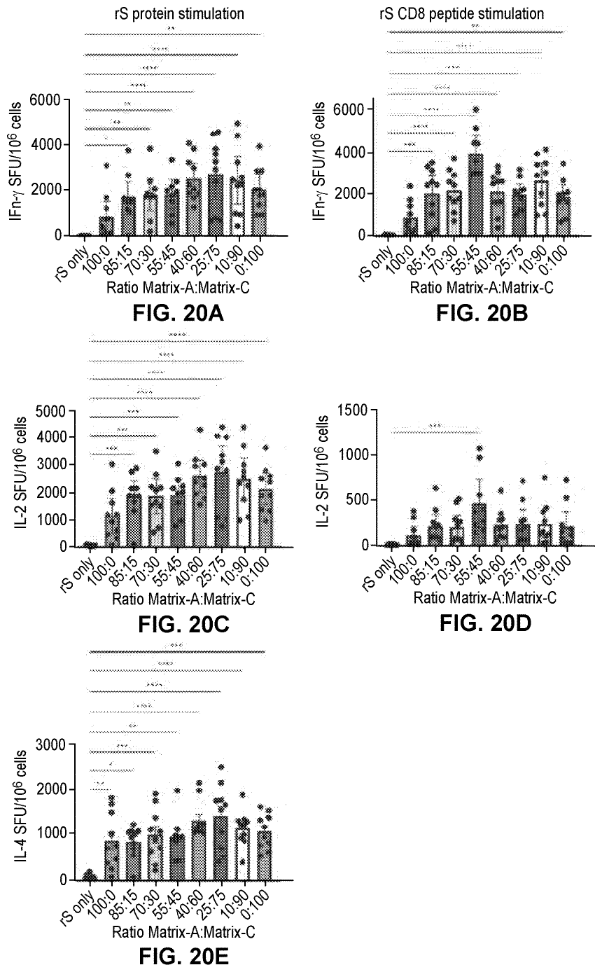

Antigen-specific responses detected by fluorospot assay showed either total T cells in a restimulation with whole rS protein or CD8+ T-cells using the highly conserved and immunodominant SARS-CoV-1 and SARS-CoV-2 Spike protein-derived CD8 peptide VNFNFNGL (Poluektov, George et al., 2021). IFN-γ, IL-2, and IL-4 production by total T cells (rS protein restimulation) was detected in an adjuvant dependent manner with significantly increased responses across the entire Matrix-A:Matrix-C ratio range compared to the antigen only group (rS) (FIGS. 20A, 20C, 20E). Analyzing antigen-specific CD8+ T-cell responses, an adjuvant effect on the generation of IFN-γ producing cells was found for Matrix-A:Matrix:C ratios ranging from 85:15 to 0:100 (FIG. 20B). For IL-2 responses by CD8+ T cells, significant differences to the antigen only group were detected for the group receiving rS immunization adjuvanted with the Matrix-A:Matrix-C ratio of 55:45 (FIG. 20D).

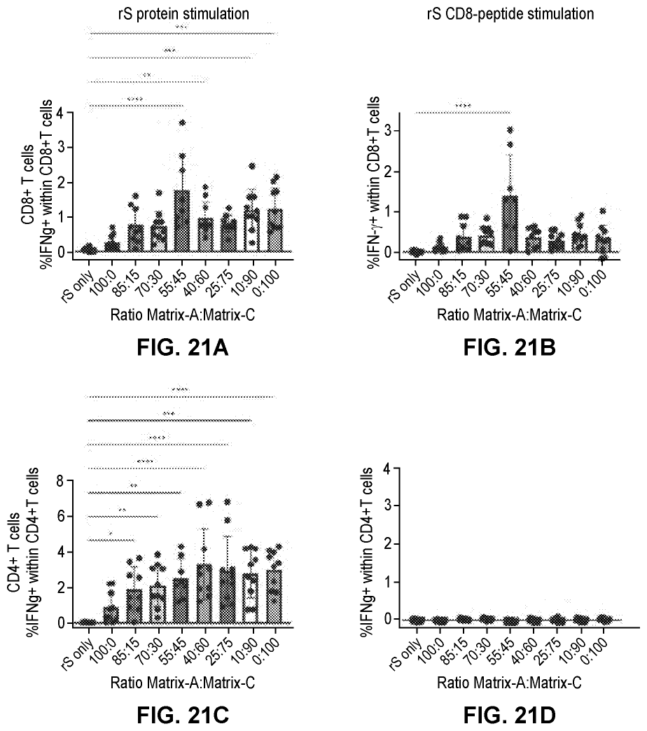

As a complementary method that allows to distinguish between antigen-specific CD4+ and CD8+ T-cell subsets, intracellular staining followed by flow cytometry analysis was performed. Splenocytes were restimulated as above. rS protein stimulation showed significantly elevated antigen-specific IFN-γ CD8+ T-cell responses for the range of Matrix-A:Matrix-C ratios from 55:45 to 0:100 (FIG. 21A). rS peptide stimulation showed significantly elevated antigen-specific IFN-γ CD8+ T-cell responses for the range of Matrix-A:Matrix-C ratio of 55:45 (FIG. 21B).

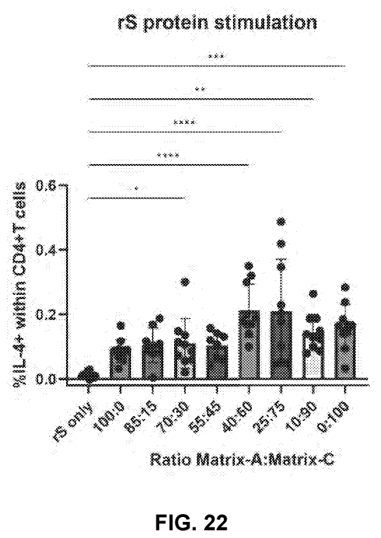

Looking at CD4+ T-cell responses, an adjuvant dependent induction of responses was found. Responses were significantly elevated compared to the antigen only group for the Matrix-A:Matrix-C ratio ranging from 85:15 to 0:100 for IFN-γ (FIGS. 21C, 21D) and for 70:30 to 0:100 for IL-4 (FIG. 22).

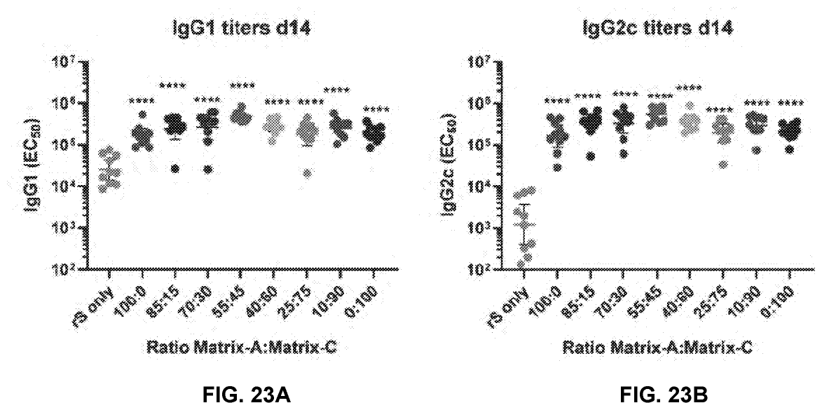

In addition to cellular responses, the generation of rS-specific IgG1 and IgG2c antibodies was evaluated in serum by ELISA (FIGS. 23A, 23B). All Matrix adjuvanted groups from Matrix-A:Matrix-C 100:0 to 0:100 showed significantly elevated rS-specific antibody titers compared to the antigen only (rS) group.

Altogether, the results show that a broad range for Matrix-A:Matrix-C ratios provided strong adjuvant activity for the rS vaccine. All tested ratios significantly enhanced rS-specific antibody responses. For cellular responses, a range of 85:15 to 0:100 could demonstrate significant effects compared to antigen alone.

1. A saponin matrix composition comprising Matrix-A particles and Matrix-C particles, wherein the Matrix-A particles and the Matrix-C particles are present at a fixed weight ratio in the range of 70:30 to 10:90 of the Matrix-A particles to the Matrix-C particles.

2. The saponin matrix composition according to claim 1, wherein the fixed weight ratio of the Matrix-A particles to the Matrix-C particles is about 60:40, 59:41, 58:42, 57:43, 56:44, 55:45, 54:46, 53:47, 52:48, 51:49, or 50:50.

3. The saponin matrix composition according to claim 1, wherein the composition, when administered together with an antigen to a subject, induces an increased production of IFN-γ by antigen-specific CD8 T cells under comparable dosing and administration conditions compared to a saponin matrix composition having a weight ratio of 85:15 of the Matrix-A particles to the Matrix-C particles.

4. The saponin matrix composition according to claim 1, wherein, when used as an adjuvant for immunization of a mouse with a SARS-CoV-2 rS protein antigen, the composition leads to an elevated frequency of antigen-specific CD8 T-cell IFN-γ responses as detected ex vivo by restimulation of splenocytes from the immunized mouse with the antigen, compared to a saponin matrix composition having a weight ratio of 85:15 of the Matrix-A particles to the Matrix-C particles under comparable immunization conditions.

5. The saponin matrix composition according to claim 1, wherein Matrix-A particles and Matrix-C particles are prepared from saponin Fraction-A and saponin Fraction-C of Quillaja saponaria Molina obtained by reversed-phase high-performance liquid chromatography of a purified Quillaja saponin extract, followed by formation of lipid-containing particles with cholesterol and phosphatidylcholine.

6. The saponin matrix composition according to claim 1, wherein the Matrix-A particles and the Matrix-C particles are provided in an aqueous composition in phosphate buffer including sodium chloride and potassium chloride at a pH of about 7.2.

7. The saponin matrix composition according to claim 1, wherein the composition is provided as an aqueous composition that has been subjected to spray drying or lyophilization to yield a solid composition, the solid composition being formulated into a solid dosage form or reconstituted in a pharmaceutically acceptable liquid vehicle to provide a liquid dosage form suitable for administration to a subject.

8. The saponin matrix composition according to claim 1, wherein administration of the composition together with an antigen to a subject results in increased antigen cross-presentation compared to administration of a saponin matrix composition having a weight ratio of 85:15 of the Matrix-A particles to the Matrix-C particles.

9. The saponin matrix composition according to claim 1, wherein administration of the composition together with an antigen to a subject results in increased antigen cross-presentation compared to administration of a saponin matrix composition having a weight ratio of 85:15 of the Matrix-A particles to the Matrix-C particles, and wherein the increased antigen cross-presentation in the subject involves increased induction of lysosomal membrane permeabilization in antigen-presenting cells compared to administering a vaccine composition comprising the antigen and a saponin matrix composition having a weight ratio of 85:15 of the Matrix-A particles to the Matrix-C particles under comparable dosing and administration conditions.

10. The saponin matrix composition according to claim 1, wherein administration of the composition together with an antigen to a subject results in increased activation of antigen-presenting cells compared to administration of a saponin matrix composition having a weight ratio of 85:15 of the Matrix-A particles to the Matrix-C particles.

11. The saponin matrix composition according to claim 1, wherein the composition, when administered together with an antigen to a subject, induces an increase in antigen-specific CD8 T-cell responses by at least 30%, by at least 50%, by at least 75%, or by at least 100%, relative to a saponin matrix composition having a weight ratio of 85:15 of the Matrix-A particles to the Matrix-C particles when administered under comparable conditions.

12. The saponin matrix composition according to claim 1, wherein the composition is for use as an adjuvant in a vaccine composition comprising a protein antigen selected from a SARS-CoV-2 S protein antigen, a respiratory syncytial virus F protein antigen, a parainfluenza virus type 3 antigen, or a human metapneumovirus F antigen.

13. The saponin matrix composition according to claim 1, wherein the composition is an aqueous composition, is in a lyophilized form, or is in a spray dried form.

14. A method for inducing production of IFN-γ by antigen-specific CD8 T cells in a subject in need thereof, the method comprising administering to the subject an effective amount of an antigen and an effective amount of a saponin matrix composition of claim 1, thereby inducing production of IFN-γ by antigen-specific CD8 T cells in the subject.

15. The method according to claim 14, wherein administering the antigen and the saponin matrix composition to the subject induces an increased production of IFN-γ by antigen-specific CD8 T cells compared to administering a vaccine composition comprising the antigen and a saponin matrix composition having a weight ratio of 85:15 of the Matrix-A particles to the Matrix-C particles under comparable dosing and administration conditions.

16. The method according to claim 14, wherein the antigen is a viral antigen selected from a SARS-CoV-2 S protein antigen, a parainfluenza virus type 3 antigen, a respiratory syncytial virus F0 antigen, and a human metapneumovirus F antigen.

17. The method according to claim 14, wherein the antigen is a tumor neoantigen selected from KRAS G12D, KRAS G12V, KRAS G12C, KRAS G12A, KRAS G13D, BRAF V600E, PIK3CA H1047R, TP53 R175H, TP53 R273H, SYT-SSX1, SYT-SSX2, PAX3-FOX01, EWS-FLI1, BCR-ABL b3a2, BCR-ABL b2a2, CCDC6-RET, FGFR3-TACC3, TPM3-ALK, TPM4-ALK, NTRK1 fusion TPM3-NTRK1, ETV6-NTRK3, DEK-AFF2, TGFBR2 frameshift-1A, ACVR2A frameshift-1A, CASP5 frameshift, TAFIB frameshift, ASTE1/HT001 frameshift, or wherein the antigen is a tumor-associated antigen selected from HPV-16 E6, HPV-16 E7, HPV-18 E6, HPV-18 E7, melanoma-associated antigen MAGE family antigens, and prostate-specific antigen PSA.

18. The method according to claim 14, wherein the antigen is present at a fixed weight ratio from 0.1 μg to 200 μg of the antigen per 400 μg to 20 μg of the total weight of the Matrix-A particles and the Matrix-C particles.

19. The method according to claim 14, wherein the antigen is present at a fixed weight ratio from 5 μg to 100 μg of the antigen per 400 μg to 50 μg of the total weight of the Matrix-A particles and the Matrix-C particles.

20. The method according to claim 14, wherein administering the antigen and the saponin matrix composition to the subject results in an increase in the frequency of antigen-specific CD8 T cells producing IFN-γ by at least 30%, specifically at least 50%, more specifically at least 75%, more specifically at least 100%, relative to administering a vaccine composition comprising the antigen and a saponin matrix composition having a weight ratio of 85:15 of the Matrix-A particles to the Matrix-C particles under comparable dosing and administration conditions.

21. The method according to claim 14, wherein administering the antigen and the saponin matrix composition to the subject elicits increased antigen cross-presentation in antigen-presenting cells compared to administering a vaccine composition comprising the antigen and a saponin matrix composition having a weight ratio of 85:15 of the Matrix-A particles to the Matrix-C particles.

22. The method according to claim 14, wherein the antigen and the saponin matrix composition are co-formulated in a single vaccine composition that is administered to the subject as an intramuscular injection.

23. The method according to claim 14, wherein the antigen and the saponin matrix composition are administered to the subject in separate pharmaceutical compositions given at the same anatomical site within a time window of up to 24 hours.

24. The method according to claim 14, wherein the subject is selected from a cow, horse, pig, sheep, or human.
